SEQUENCE LISTING (57 sequences)

SEQ ID NO: 1 - DNA, 35 bp, Artificial Sequence
  aptamer
cgtaaatcagtcagaaggcgtggtaccacgctttc

SEQ ID NO: 2 - DNA, 56 bp, Artificial Sequence
  aptamer
tgactgatttacggaagctgaataaggactgcttaggattgcgatgattcagcttc

SEQ ID NO: 3 - DNA, 35 bp, Artificial Sequence
  aptamer
cgtaaatcagtcagaagtcgacgccggtcgacttc

SEQ ID NO: 4 - DNA, 56 bp, Artificial Sequence
  aptamer
tgactgatttacggaagttacggacggatgtcagtggtatagtaatccgtaacttc

SEQ ID NO: 5 - DNA, 56 bp, Artificial Sequence; identical to SEQ ID NO: 2
  aptamer

SEQ ID NO: 6 - DNA, 56 bp, Artificial Sequence
  aptamer
cgtaaatcagtcagaagctgaataaggactgcttaggattgcgatgattcagcttc

SEQ ID NO: 7 - DNA, 91 bp, Artificial Sequence
  aptamer
cgtaaatcagtcagaaggcgtggtaccacgctttctgactgatttacggaagctgaataaggactgcttaggattgcgatgattcagcttc

SEQ ID NO: 8 - DNA, 91 bp, Artificial Sequence
  aptamer
tttttttttttttgaaggcgtggtaccacgctttctttttttttttttgaagctgaataaggactgcttaggattgcgatgattcagcttc

SEQ ID NO: 9 - DNA, 14 bp, Artificial Sequence
  aptamer
gcgtggtaccacgc

SEQ ID NO: 10 - DNA, 66 bp, Artificial Sequence
  aptamer
cagcaccgtcaactgaataaggactgcttaggattgcgatgattcagggtgatgcgattgagatgt

SEQ ID NO: 11 - DNA, 35 bp, Artificial Sequence
  aptamer
ctgaataaggactgcttaggattgcgatgattcag

SEQ ID NO: 12 - DNA, 35 bp, Artificial Sequence; identical to SEQ ID NO: 1
  aptamer

SEQ ID NO: 13 - DNA, 56 bp, Artificial Sequence; identical to SEQ ID NO: 2
  aptamer

SEQ ID NO: 14 - DNA, 56 bp, Artificial Sequence; identical to SEQ ID NO: 6
  aptamer

SEQ ID NO: 15 - DNA, 91 bp, Artificial Sequence
  aptamer
tttttttttttttgaagctgaataaggactgcttaggattgcgatgattcagcttctttttttttttttgaaggcgtggtaccacgctttc

SEQ ID NO: 16 - protein, 14 aa, Artificial Sequence
  tau peptide
GYSSPGSPGTPGSR

SEQ ID NO: 17 - protein, 13 aa, Artificial Sequence
  tau peptide
KVAVVRTPPKSPS

SEQ ID NO: 18 - protein, 13 aa, Artificial Sequence
  tau peptide
KVAVVRTPPKSPS

SEQ ID NO: 19 - DNA, 51 bp, Artificial Sequence
  aptamer
aggatagggggtagctcggtcgtgtttttgggttgtttggtgggtcttctg

SEQ ID NO: 20 - DNA, 21 bp, Artificial Sequence
  primer
tcacctgagacttgacgatgg

SEQ ID NO: 21 - DNA, 18 bp, Artificial Sequence
  primer
tggacagacgatagcact

SEQ ID NO: 22 - DNA, 41 bp, Artificial Sequence
  primer
  misc_feature (22)..(22): n is a, c, g, or t
tcacctgagacttgacgatggngagtgctatcgtctgtcca

SEQ ID NO: 23 - DNA, 91 bp, Artificial Sequence
  aptamer
tgactgatttacggaagctgaataaggactgcttaggattgcgatgattcagcttccgtaaatcagtcagaaggcgtggtaccacgctttc

SEQ ID NO: 24 - DNA, 112 bp, Artificial Sequence
  aptamer
tgactgatttacggaagctgaataaggactgcttaggattgcgatgattcagcttccgtaaatcagtcagaagctgaataaggactgcttaggattgcgatgattcagcttc

SEQ ID NO: 25 - DNA, 76 bp, Artificial Sequence
  aptamer
tcacctgagacttgacgatggcatcactcccccccacctattacatcatcataaattgagtgctatcgtctgtcca

SEQ ID NO: 26 - DNA, 76 bp, Artificial Sequence
  aptamer
tcacctgagacttgacgatggcctccccctcacgcactcttccgtttcttcttatctgagtgctatcgtctgtcca

SEQ ID NO: 27 - DNA, 76 bp, Artificial Sequence
  aptamer
tcacctgagacttgacgatggaactccccccaccattatcagcgcaccaccattgtagagtgctatcgtctgtcca

SEQ ID NO: 28 - DNA, 76 bp, Artificial Sequence
  aptamer
tcacctgagacttgacgatggtttaactcccccacgccccccgccaacccatctccagagtgctatcgtctgtcca

SEQ ID NO: 29 - DNA, 75 bp, Artificial Sequence
  aptamer
tcacctgagacttgacgatggtacgacggccccccgattatgcgactacttgatttgagtgctatcgtctgtcca

SEQ ID NO: 30 - DNA, 76 bp, Artificial Sequence
  aptamer
tcacctgagacttgacgatggtcagaacgacgcgccccccacctcattcattattttgagtgctatcgtctgtcca

SEQ ID NO: 31 - DNA, 76 bp, Artificial Sequence
  aptamer
tcacctgagacttgacgatggtgaccaccccccacgcacacacacctcttccatcctgagtgctatcgtctgtcca

SEQ ID NO: 32 - DNA, 76 bp, Artificial Sequence
  aptamer
tcacctgagacttgacgatggcactacccctccctactaagcacggtatcttgtactgagtgctatcgtctgtcca

SEQ ID NO: 33 - DNA, 76 bp, Artificial Sequence
  aptamer
tcacctgagacttgacgatgggaacaaacaccgcgaccacccccccacttaactcctgagtgctatcgtctgtcca

SEQ ID NO: 34 - DNA, 76 bp, Artificial Sequence
  aptamer
tcacctgagacttgacgatggcaatccccccgacaccgaatcctaagcgaacaacgcgagtgctatcgtctgtcca

SEQ ID NO: 35 - DNA, 76 bp, Artificial Sequence
  aptamer
tcacctgagacttgacgatggactcacaaactcgagccacccccgacccacacaacagagtgctatcgtctgtcca

SEQ ID NO: 36 - DNA, 76 bp, Artificial Sequence
  aptamer
tcacctgagacttgacgatggtactcccccccaacctaatagctctttaccctctgagagtgctatcgtctgtcca

SEQ ID NO: 37 - DNA, 76 bp, Artificial Sequence
  aptamer
tcacctgagacttgacgatggccgactccccaccctacatcgcaacattgactattagagtgctatcgtctgtcca

SEQ ID NO: 38 - DNA, 76 bp, Artificial Sequence
  aptamer
tcacctgagacttgacgatggttctacactgcccccccgacccgccagaccaacccagagtgctatcgtctgtcca

SEQ ID NO: 39 - DNA, 76 bp, Artificial Sequence
  aptamer
tcacctgagacttgacgatggcaatcctccgagctccacccacccttactcaacattgagtgctatcgtctgtcca

SEQ ID NO: 40 - DNA, 76 bp, Artificial Sequence
  aptamer
tcacctgagacttgacgatggcgctaccccctaacttcaacccgcattattctagctgagtgctatcgtctgtcca

SEQ ID NO: 41 - DNA, 76 bp, Artificial Sequence
  aptamer
tcacctgagacttgacgatggttaccgaacccgacacccccgccgacaccagccccagagtgctatcgtctgtcca

SEQ ID NO: 42 - DNA, 76 bp, Artificial Sequence
  aptamer
tcacctgagacttgacgatggcccccccccgcaccgcctcattcagcatactaatacgagtgctatcgtctgtcca

SEQ ID NO: 43 - DNA, 76 bp, Artificial Sequence
  aptamer
tcacctgagacttgacgatggcaaccaccccccctggctacatcatattcttatcttgagtgctatcgtctgtcca

SEQ ID NO: 44 - DNA, 76 bp, Artificial Sequence
  aptamer
tcacctgagacttgacgatggtttcttcgccccccccacacactacacgtttcttctgagtgctatcgtctgtcca

SEQ ID NO: 45 - DNA, 76 bp, Artificial Sequence
  aptamer
tcacctgagacttgacgatggttacctcacacccccccgcacgcactacccccgcccgagtgctatcgtctgtcca

SEQ ID NO: 46 - DNA, 76 bp, Artificial Sequence; identical to SEQ ID NO: 28
  aptamer

SEQ ID NO: 47 - DNA, 76 bp, Artificial Sequence; identical to SEQ ID NO: 31
  aptamer

SEQ ID NO: 48 - DNA, 76 bp, Artificial Sequence
  aptamer
tcacctgagacttgacgatggtctaattcgcaccccccctctactcgccaccccccagagtgctatcgtctgtcca

SEQ ID NO: 49 - DNA, 76 bp, Artificial Sequence
  aptamer
tcacctgagacttgacgatggtgttgttttccaccctactcgccgcccccccgacccgagtgctatcgtctgtcca

SEQ ID NO: 50 - DNA, 76 bp, Artificial Sequence
  aptamer
tcacctgagacttgacgatggtaacgaaccgcgcccaccacacgcccacccccccacgagtgctatcgtctgtcca

SEQ ID NO: 51 - DNA, 76 bp, Artificial Sequence
  aptamer
tcacctgagacttgacgatggtccacgttaccgcacacccgccgacacccccgcctcgagtgctatcgtctgtcca

SEQ ID NO: 52 - DNA, 76 bp, Artificial Sequence
  aptamer
tcacctgagacttgacgatggtgaccaccccccacgcacacacaccccttccatcctgagtgctatcgtctgtcca

SEQ ID NO: 53 - DNA, 76 bp, Artificial Sequence
  aptamer
tcacctgagacttgacgatggactaccgcaccaccaccacacccccccgaccgcactgagtgctatcgtctgtcca

SEQ ID NO: 54 - DNA, 76 bp, Artificial Sequence
  aptamer
tcacctgagacttgacgatggtaacctcacccgcatccccccccagttcaattattagagtgctatcgtctgtcca

SEQ ID NO: 55 - DNA, 66 bp, Artificial Sequence
  aptamer
gacttgacgatggcatcactcccccccacctattacatcatcataaattgagtgctatcgtctgtc

SEQ ID NO: 56 - DNA, 57 bp, Artificial Sequence
  aptamer
gacgatggcatcactcccccccacctattacatcatcataaattgagtgctatcgtc

SEQ ID NO: 57 - DNA, 57 bp, Artificial Sequence
  aptamer
gacgatagcatcactcccccccacctattacatcatcataaattgagtgctatcgtc

(19) United States
(12) Patent Application Publication
(10) Pub. No.: US 2023/0159935 A1
(43) Pub. Date: May 25, 2023

(54) CIRCULAR BIFUNCTIONAL APTAMERS AND TRIFUNCTIONAL APTAMERS TARGETING Tau

(71) Applicants: UNIVERSITY OF FLORIDA RESEARCH FOUNDATION, INCORPORATED, Gainesville, FL (US); GRYPHON BIO THERAPEUTICS, INC., South San Francisco, CA (US)

(72) Inventors: Kevin K-Wang WANG, Gainesville, FL (US); Xiaowei LI, Gainesville, FL (US); Weihong TAN, San Ranon, CA (US); Bang WANG, Gainesville, FL (US); William HASKINS, South San Francisco, CA (US)

(21) Appl. No.: 17/797,217

(22) PCT Filed: Mar. 1, 2021
(86) PCT No.: PCT/US2021/020322

§ 371 Date: Aug. 3, 2022

Related U.S. Application Data
(60) Provisional application No. 62/983,173, filed on Feb. 28, 2020.

(51) Int. Cl. C12N 15/115

(52) U.S. Cl. CPC C12N 15/115; C12N 2310/16; C12N 2310/3513; C12N 2310/3517; C12N 2310/3519; C12N 2310/51; C12N 2310/532

(57) ABSTRACT

The lack of blood-brain barrier (BBB) penetrating ability has hindered the delivery of many therapeutic agents for tauopathy therapeutic treatment. A circular bifunctional aptamer reported here has been able to enhance the in vivo BBB penetration for improved therapy. The circular aptamer includes one transferrin receptor (TfR) aptamer to facilitate TfR-aptamer recognition-induced transcytosis across BBB endothelial cells, and one Tau protein aptamer selected to inhibit Tau phosphorylation and other tauopathy-related pathological events in the brain. This bispecific construct exhibits strong specificity towards Tau and enhanced plasma stability in comparison to linear Tau aptamer. In vivo administration of circular Tau-TfR aptamer results in a rapid uptake into relevant brain regions after crossing the BBB, such as hippocampus and cortex. A Y-shaped trispecific aptamer including one aptamer for L1CAM, one aptamer for Tau and one aptamer for TfR reported here has enhanced BBB and neuron cell membrane permeation. Bispecific and trispecific Tau aptamer coupled to a signaling moiety (such as dodecane tetraacetic acid (DOTA) or DOTA complexed to Gd+3) for neuroimaging, and bispecific or trispecific Tau aptamer coupled to protein aggregate binding moiety (such as methylene blue) for enhanced ability to disrupt tau aggregation are also contemplated in this invention.

### GOVERNMENT FUNDING SUPPORT

This invention was made with government support under grant no. W81XWH1910860 awarded by U.S. Assistant Secretary of Defense for Health Affairs endorsed by the U.S. Department of Defense. The government has certain rights in the invention.

### FIELD

The disclosed invention relates generally to products and methods useful for treating and diagnosing tauopathy-related neurodegenerative disorders.

### BACKGROUND

Tau protein is a microtubule associated protein enriched in the axons of neurons in the central nervous system. Tau is known to promote the assembly of microtubules to maintain microtubule integrity in neurons and contribute to axonal outgrowth. Evidence has confirmed that the accumulation of unregulated hyperphosphorylation and other post-translational modifications can convert Tau from an important multifunctional protein to a neurotoxic entity, triggering Tau oligomerization/aggregation and resulting in neurofibrillary tangles (NFTs) and ultimately to irreversible neurodegeneration. Hyperphosphorylation of Tau (to form P-Tau) causes it to dissociate from the microtubule and to form aggregates and filaments, which result in death of the neurons. This phenomenon is termed tauopathy and is found to be pathologically involved in several neurodegenerative disorders.

Tauopathies, including traumatic brain injury (TBI), chronic traumatic encephalopathy (CTE), Alzheimer's disease (AD), frontotemporal dementia, primary age-related tauopathy (PART), frontotemporal dementia (FTD), Parkinsonism linked to chromosome 17 (FTDPL-17), and others, are a group of neurodegenerative diseases characterized by the abnormal deposition of phosphorylated Tau (P-Tau) filaments. The common causes for these conditions are unregulated Tau hyperphosphorylation and other post-translational modifications that in turn lead to Tau oligomerization/aggregation, resulting over time to neuronal death and irreversible neurodegeneration.

There are almost 1.9 million new cases of traumatic brain injury (TBI) incidents each year in the United States. In addition, over 5 million of the United States population might be living with some form of chronic issues due to traumatic brain injury (TBI). It is increasingly recognized that TBI is a complex, heterogeneous disorder. For example, repetitive concussion or mild TBI can result in brain protein (Tau) aggregate accumulation over time, leading to neurodegenerative condition called chronic traumatic encephalopathy (CTE).

Levels of total Tau and phosphorylated Tau (P-Tau) in cerebrospinal fluid (CSF) highly correlate to the progression of Alzheimer's disease. However, current methods to detect total Tau and P-Tau, including mass spectrometry, positron emission tomography (PET) imaging, and western blotting in combination with immunoprecipitation or high-performance liquid chromatography (HPLC), usually require a large amount of sample (i.e., brain tissue or CSF) to overcome the detection limit and are either too costly or too tedious to operate.

So far, many Tau-directed therapeutic interventions have been proposed for treating tauopathies, including inhibiting Tau kinase or stimulating phosphatase to suppress P-Tau, administering Tau/P-Tau antibodies for immunotherapy, and stabilizing microtubules or preventing Tau aggregation using chemotherapy drugs. However, it may not be sufficient to target only one kinase or phosphatase since many are involved in the hyperphosphorylation process. Controlling antibody quality and production consistency is also challenging and expensive. In addition, Tau aggregation inhibitors (e.g., methylene blue) have been reported to lack targeting specificity because they bind to the hydrophobic domains of multiple proteins. More importantly, the blood-brain barrier (BBB) is a major obstacle that limits the transport, and therefore the effectiveness, of many therapeutic antibodies and drug candidates (e.g., the microtubule-stabilizer paclitaxel). In summary, despite the emergence of potential tauopathy-related theranostic probes, the BBB remains a great obstacle, restricting their brain deliveries. The lack of specificity among drug candidates further reduces the correct transport into targeted brain regions and weakens therapeutic effects.

Therefore, there is a need in the market for a tool and method to detect Tau and P-Tau for diagnostic purposes, and a need in the market for a tool and method for use in treatment methods as well. Currently, there is no Tau-binding agent DNA aptamer on the market.

The problems of current approaches to the treatment of tauopathies include low specificity towards Tau protein, difficult quality control and inefficient BBB penetration. Aptamers capable of selective binding to defined targets are being considered as an alternative therapeutic strategy. Chemically synthesized aptamers can be reproducibly scaled-up, and they are more readily modified with functional groups to meet specific therapeutic and diagnostic needs. Aptamer probes relevant to the central nervous system (CNS) display protective benefits in the context of neuronal functions or neurodegeneration, as well.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 illustrates Human Tau 441 structure. Number indicates the major known phosphorylation sites (e.g. S202=ser 202; T231=Th 231).

FIG. 2 is a schematic showing the structures of the indicated TfR and Tau aptamers: TfR aptamer (FIG. 2A), TfR-13 aptamer (FIG. 2B), IT2a aptamer (FIG. 2C), and IT2a-13 aptamer (FIG. 2D).

FIG. 3 is a schematic showing the structures of linear (FIG. 3A) and circularized (FIG. 3B) TfR/IT2a Tau bifunctional aptamers.

FIG. 4 is a schematic showing the structures of non-circular IT2a-13 aptamer (FIG. 4A) and circularized IT1a/cIT2a dimeric Tau aptamer (FIG. 4B).

FIG. 5A and FIG. 5B are schematics showing the construction of the circular bispecific Tau-L1CAM aptamer, Tau-GLAST-1 Aptamer, or Circular Tau-ACSA-2 Aptamer (FIG. 5A) and the Y-shaped trispecific Tau-TfR-L1CAM aptamer, Tau-TfR-GLAST-1 Aptamer, or Tau-TfR-ACSA-2 Aptamer (FIG. 5B).

FIG. 6A shows examples of Tau aptamer coupling linkers to methylene blue, including -(PEG)ₙ, -poly-T-, and —S—S— linkers. FIG. 6B shows an example of circular Tau-TfR bispecific aptamer coupling to methylene blue.

FIG. 7A is a schematic showing the production of the circular bifunctional aptamer, which incorporates transferrin receptor (TfR) aptamer and Tau protein aptamer IT2a. FIG. 7B is a schematic of the transcytosis of the circular aptamer crossing the blood-brain barrier via specific recognition between TfR and TfR aptamer. FIG. 7C is an image of an agarose gel analysis of the circular aptamer and its components.

FIG. 8A, FIG. 8B, and FIG. 8C are 4% agarose gels showing the stability tests of IT2a aptamer and circular Tau-TfR aptamer.

FIG. 9A and FIG. 9B are 12% urea PAGE gels that show the serum stability tests of circular aptamers as indicated.

FIG. 10 is a set of 4% agarose gels showing the stability of IT2a aptamer and circular aptamer in mouse plasma (FIG. 10A) and in mouse brain lysate (FIG. 10B).

FIG. 11A is a graph showing normalized intensity after flow cytometry analysis of the indicated aptamers to become internalized in target bEnd.3 cells. FIG. 11B is a graph showing normalized intensity after flow cytometry analysis of circular aptamer incubated for the indicated times.

FIG. 12A, FIG. 12B, FIG. 12C, and FIG. 12D show flow cytometry histograms demonstrating the specific binding tests of the indicated aptamers on control Ni beads (FIG. 12A), T231-Ni beads (FIG. 12B), T231P-Ni beads (FIG. 12C), and S202 beads (FIG. 12D).

FIG. 13A and FIG. 13B are non-denaturing gels stained with Coomassie Brilliant Blue (FIG. 13A) and aptamer fluorescence (FIG. 13B).

FIG. 14 presents data on flow cytometry and confocal microscopy analysis. FIG. 14A and FIG. 14B show that circular aptamer selectively bound to TfR-positive bEnd.3 cells but not TfR-negative HEK293 cells.

FIG. 15 is a set of images showing that the circular aptamer was specifically internalized into target bEnd.3 cells, but not negative HEK293 cells.

FIG. 16 is a schematic showing a circular aptamer crossing in an in vitro BBB model.

FIG. 17 is a set of graphs showing the transport efficiency of TAMRA-labeled dextran controls (70 kDa, 10 kDa) across the BBB in vitro (FIG. 17A) and of all samples as indicated across the transwell membrane only (FIG. 17B).

FIG. 18A and FIG. 18B are graphs showing the transport efficiency of IT2a aptamer, TfR aptamer, circular aptamer and other control samples, as indicated, in the BBB model. FIG. 18C is a bar graph showing the transport ratio (%) of each sample aptamer, as indicated.

FIG. 19 is a set of bar graphs presenting data from the cell viability tests conducted with bEnd.3 cells (FIG. 19A) and SY5Y0SH cells (FIG. 19B), incubated with TfR aptamer, Tau aptamer, and circular aptamer at different concentrations.

FIG. 20A and FIG. 20B are ex vivo fluorescent organ images of mice injected with Cy5.5-labeled circular Tau-TfR aptamer, Tau aptamer, and TfR aptamer for different time periods.

FIG. 21A is a bar graph showing quantitative analysis of the normalized fluorescent signal of the brains. FIG. 21B is a graph showing the blood circulation time of Cy5.5-labeled aptamers in vivo.

FIG. 22A, FIG. 22B, FIG. 22C, FIG. 22D, and FIG. 22E (inset of FIG. 22D) are confocal images of entire dissected brain slices from mice treated with Cy5.5-labeled aptamers for 1 hour. In FIG. 22D, red colored spots are the brain areas showing staining of Cy5.5-Circular Tau-TfR Aptamer. Representative of three separate experiments. Scale bar: 200 μm. In FIG. 22E, red colored spots are the brain areas showing staining of Cy5.5-Circular Tau-TfR Aptamer with DNA (DARPI) staining (blue) as counterstaining. Area shown aptamer labeling included cortex (Ctx), corpus callosum (CC), hippocampus (CA1 and dentate gyrus (DG) regions) and subventricular zone (SVZ). Representative of three separate experiments. Scale bar: 200 μm

FIG. 23 is a set of bar graphs showing results of quantitative analysis of the normalized fluorescent signal from heart (FIG. 23A), spleen (FIG. 23B), kidney (FIG. 23C), liver (FIG. 23D), and lung (FIG. 23E).

FIG. 24 shows confocal fluorescence partial brain slice images of mice treated with Cy5.5-labeled Tau aptamer (FIG. 24A and FIG. 24B), TfR aptamer (FIG. 24C and FIG. 24D), or circular Tau-TfR aptamer (FIG. 24E and FIG. 24F) for 1 hour. Scale bar: 40 μm. Solid arrows indicate DNA staining with DAPI, dotted arrows indicate respective aptamer labeling.

FIG. 25 shows methylene blue (MB) coupling to Tau aptamer IT2a and circular Tau-TfR bispecific aptamer, including the conjugation steps (FIG. 25A) and Tau aptamer-MB binding to Tau monomer, suppressing oligomer formation of preventing oligomer forming Tau aggregate (FIG. 25B). Scale bar: 40 μm. Solid arrows indicate DNA staining with DAPI, dotted arrows indicate respective aptamer labeling.

FIG. 26 is a schematic showing circular Tau-TfR1 bispecific aptamer coupling to Gd⁺³ chelating molecule (DOTA-mono NHS tris ester) to form a dodecane tetraacetic acid (DOTA)-circular aptamer.

FIG. 27 is a flow chart showing the TBI mouse set-up, aptamer treatments, and biomarkers collection for ELISA.

FIG. 28A through FIG. 28D show that circular Tau-TfR aptamer attenuated the protein levels of TBI-related T-Tau (FIG. 28A), P-Tau (FIG. 28B), GFAP (FIG. 28C), and pNF-H (FIG. 28D) in both injured and control brain tissues after CCI.

FIG. 29 is a bar graph presenting data from an ELISA assessment of Tau protein levels in the serum of untreated or aptamer treated hTau mice after CCI surgeries.

FIG. 30 is a drawing showing the operation of the acquisition trial and the retrieval trial of the Y-maze behavior tests.

FIG. 31A is a schematic timeline of the FPI model, circular aptamer injection and Y-maze test. FIG. 31B is a graph showing the results of the Y-maze tests.

FIG. 32 is a schematic illustration of the two step Functional SELEX process and Tau aptamers functional validation workflow.

FIG. 33A is the sequence of forward primer and reverse primer. FIG. 33B is an amplification plot for a 10-fold dilution random library template series by real-time PCR. FIG. 33A pertains to the DNA library containing 36 nucleotide random sequences and the sequence of forward primer and reverse primer. FIG. 33B shows an amplification plot for a 10-fold dilution random library template series by real-time PCR. The amplification began with a hot start at 95deg. C. for 90 s to activate Tag DNA polymerase. Then each of the repeated amplification cycles was performed at 95deg. C. for 5 s, 57deg. C. for 30 s and 72deg. C. for 30 s. No template control (NTC) show a positive signal after 30 cycles incidating formation f primer dimer.

FIG. 34 is a table of the 13th round Tau functional SELEX sequencing results referred to as the BW series. These were identified using 5′ACCCTAACTGACACACATTCC-(35N)-GGATGTCAGAATGCCATTTGC-3′.

FIG. 35 is a table of the 19th round functional SELEX sequencing results referred to as the K series. Starting from K1 to K10, these sequences represent SEQ ID NOs:45-54, respectively

FIG. 36 shows the two most stable predicted secondary structures of KW6 using mFold.

FIG. 37 is a polyacrylamide gel showing the binding analysis of each FAM-labeled aptamer by the gel mobility shift assay. Binding analysis of each FAM-labled aptamer by the gel mobility shift assay. Except for the first channel, each aptamer (200 nM) was incubated with Tau protein (0.02 mg/mL) at 37deg. C. for 30 min. and the complexes were separated from the free DNA on native 8% polyacrylamide gels with electrophoresis 25deg. C. and 145 V for 40 min. The DNA bands on the gels were scanned using a Typhoon Imagin System (Amersham Biosciences).

FIG. 38A is the flow cytometry results analyzing primary binding between aptamer candidates and Tau 441. FIG. 38B is a dot blot assay showing aptamer binding specificity. FIG. 38A Primary binding analysis between aptamer candidates and Tau 441. FIG. 38B Aptamers binding specificity by dot blot assay. The nitrocellulose membranes were socked in methanol until becoming wet (1 min) then the membranes were washed with PBST solution for 5 mins. BSA, Casein, IgG, Tau and P-Tau (1 μg per protein) were respectively immobilized as striped dots on the NC membranes by air-drying. The membranes were blocked with 1% fat-free milk in PBST for 1 hour and incubated with heat-denatured aptamers (250 nM) labeled with FAM for 1 hour at room temperature. Control DNA were random sequence DNA with FAM label. The membranes were washed three times with TBST. Dots were scanned by Typhoon Imaging System (Amersham Biosciences).

FIG. 39 shows predicted secondary structures of the BW series using mFold.

FIG. 40 (A) Secondary structures of BW1 serial aptamers predicted by mFold. BW1a and BW1b were truncated from BW1. BW1c was modified from BW1b by replacing a G:T pair with A:T. (B) Determination of binding affinity of BW1 by dot blotting assay. (C) Primary binding analysis between aptamer candidates and Tau protein by dot blotting assay. The NC membrane was blocked with milk then incubated with each aptamer solution (100 nM). Aptamers were labeled with Cy5 at 5′ position, Dots were scanned simultaneously by a Typhoon Imaging System (Amersham Biosciences). (D) The data parameters of BW1 serial aptamers. (E) The sequences of BW1 serial aptamers (SEQ ID NOs: 25, 55, 56, and 57)

FIG. 41. (A) Schematic representation of ThT assay monitoring the process of tau aggregation with the inducer. Tau was first preincubated with the aptamer for 40 mins before adding the aggregate inducer. All the reaction were carried out at 37° C. for 12 h on a shaker. (B) Concentration: Tau441 protein: 2 uM, Aptamer: 6 uM, Arachidonic acid: 75 uM in the oligomerization buffer. (C) Concentration: Tau protein: 2 uM, Aptamer: 6 uM, Heparin: 100 μM in the oligomerization buffer. The aggregation of tau proteins was monitored in Greiner Bio-One 384-well micro-plates using a Biotek Synergy 2 microplate reader. Excitation wavelength: the excitation and emission filters were 450/15 and 485/15 nm. (D) In vitro tau phosphorylation assay was performed by incubating Tau441 (2 μM) with aptamers or tau-antibody (DA9 from mouse) (6 μM) for 30 mins, then add GSK3β (200 ng) incubating for 24 h. Samples were analyzed by SDS-PAGE, followed by Western blotting with tau antibody (DAKO A0024 from rabbit). The lower bands (blue label) is the non-phosphorylated Tau, the upper bands are phospho-Tau species (red label) (induced with GSK3B-phosphoarylation in vitro) (E) Quantification of non-phospho-Tau band (white bars) and phospho-tau bands (black bars) (expressed as % maximum). GSK3B phosphorylation induced reduction of non-phospho-Tau was reversed most effectively by DA9 Tau monoclonal antibody (MAb and by tau Aptamer BW1 and BW1c. In parallel, GSK3B phosphorylation induced elevations of phospho-Tau was reversed most effectively by DA9 Tau monoclonal antibody (MAb and by tau Aptamer BW1 and BW1c.

FIG. 42. (A) Confocal microscopy analyzing the internalization of Cy5-labeled aptamer in the N2a cells with the help of lipofectamine 2000. BW1c labeled with Cy5 incubated with N2a cells for 6 h and 24 h. (B) and (D) Cells were treated with OA (100 nM) for 24 h after the 30 mins internalization of aptamer. Immunoblots of N2a cells extracted protein (20 μg) using total Tau and phospho-tau antibodies: DA9 (Target to Total Tau), P-Tau (Ser396/5404) antibody (target to P-Tau). Different tau species are pointed with colored arrows. Blue brackets present monomeric p-tau and oligomeric p-tau. Black arrows show non-phosphorylated tau band (46 kDa). (C) Immunoblots quantification. All data are normalized to (3-actin and are expressed as a percentage of control. *p<0.05, **p<0.01, ***p<0.001 and ****p<0.0001 (n=3, two-way ANOVA).

FIG. 43. Three rounds of tau441-binding Gel Electrophoresis-SELEX (GE-SELEX) to enrich and preselect tau-binding aptamers. (A) Gel electrophoresis (GE) SELEX of tau protein. Native non-denaturing PAGE gel electrophoresis of FAM-labeled DNA pool and DNA incubated with Tau protein. Binding complexes of aptamers and tau showed a band migration and were then harvested from gel band for amplification. Unbound DNA were washed away in the electrophoresis running. (B) Flow cytometry monitored the process of GE-SELEX result. The Round 1 DNA pool already showed good binding ability with Tau protein.

FIG. 44 Tau Functional SELEX. (A) Arachidonic acids with a series of concentrations inducing tau protein aggregation have been analyzed by SDS-PAGE and the gel was stained with Coomassie blue. Concentration: Tau protein: 2 uM, Arachidonic acid: 0-100 uM, in oligomerization buffer (10 mM HEPES, 100 mM NaCl, 10 mM MgCl₂, 50 mM KCl, 1 mM EDTA, 1 mM DTT). (B) Schematic illustration of the Functional SELEX process. (C) aptamers bound to monomeric Tau were retrieved from native PAGE gel and Beads method. (D) Western blotting monitors each round of DNA pool inhibiting Tau aggregation in vitro in the process of Functional SELEX. (E) Normalized values of Oligo-Tau/Monomeric Tau protein level ratio for each selected DNA pool. Error bars represent the standard deviation.

FIG. 45. Inhibitory effects of tau-binding aptamers on aggregation of Tau441 in vitro. (A) In vitro tau oligomerization assay was performed by incubating Tau441 (2 uM) with aptamers (6 μM) for 30 mins, followed by incubation with Arachidonic acid: 75 uM. Samples were analyzed by SDS-PAGE, followed by Western blotting with Tau antibody (DA9). (B) Value quantification of oligomeric tau/monomeric tau in each band. *p<0.05, **p<0.01, ***p<0.001 and ****p<0.0001 (n=3, two-way ANOVA).

FIG. 46. Native PAGE separated the Oligomer Tau and Monomer Tau.

FIG. 47 In vitro dose-response effect of BW1 inhibit AA-induced tau oligomerization assay as detected by DA9 antibody. In vitro tau oligomerization assay was performed by incubating Tau441 (2 μM) with BW1 aptamers ((2, 4, 6 μM) for 30 mins, followed by incubation with Arachidonic acid: 50 uM. Samples were analyzed by SDS-PAGE, followed by Western blotting with Tau antibody (DA9). Then quantification of value of oligomeric tau/monomeric tau in each stripe.

FIG. 48. Cell viability test of N2a cells incubated with Lipofectamine-2000 and BW1c aptamer at different concentrations. Data are means±SD (n=3).

### DESCRIPTION

In order to achieve successful delivery of these potent Tau aptamers into tauopathic brains, BBB restriction and plasma degradation are key issues to overcome. In terms of BBB transport routes, transferrin receptor (TfR)-mediated transcytosis has been commonly used in BBB investigations, because TfRs are relatively abundant on the endothelial cells of BBB. Furthermore, both anti-TfR antibody and aptamers recognizing TfR specifically have been generated and presented efficient ability to penetrate BBB. Several epitope-specific Tau-binding DNA aptamers with high affinity were discovered by implementing SELEX (Systematic Evolution of Ligands by EXponential enrichment). The identified Tau aptamers can partially inhibit Tau hyperphosphorylation and aggregation in vitro, suggesting they can be used in vivo to disrupt tauopathies.

Based on the advantages of aptamers, a bispecific Tau-TfR-binding aptamer was produced and administered in vivo to mitigate tauopathy in the brain. To further address the problem of plasma stability for aptamer delivery in vivo, the ligation of two identical aptamers to make circular bivalent aptamers with improved in vivo stability and recognition ability (owing to unnotched and rigid DNA structures and the circular aptamer design) was also applicable in functional protein delivery. The selected Tau aptamer was optimized by ligating it with a TfR aptamer to stabilize the circular bifunctional Tau-TfR aptamer. This modified Tau-TfR bispecific aptamer continued to have strong binding ability towards Tau and exhibited enhanced plasma stability and improved BBB permeability in both in vitro and in vivo studies. Importantly, in vivo administration of a circular Tau-TfR bispecific aptamer led to attenuated brain levels of Tau and P-Tau in hTau mice subjected to TBI and negated the TBI-induced cognition/memory deficits found in hTau mice. In summary, by employing circular Tau-TfR bispecific aptamer, we show enhanced in vivo BBB penetration and brain retention via TfR protein-aptamer complex-mediated transcytosis, as well as improved attenuation of Tau levels in targeted injured brain areas and memory deficits, laying foundation for the development of specific tauopathy treatments. A trispecific aptamer with a Y-shaped structure also was produced. This aptamer binds to Tau and contains aptamers to TfR and to L1CAM allowing for enhanced in vivo BBB penetration, brain retention, and neuron cell membrane penetration.

Also described herein is a novel “Functional SELEX” system that enriched the desirable inhibitory DNA-aptamer molecules through a functionally-guided approach. Specifically, Using this principle, we were able to discovere and enrich a series of aptamer candidate (76 nucleotide-long) that preferentially bind with tau protein monomer but not tau oligomers. As a result of tau oligomerization functional SELEX, we identified a top aptamer candidate (BW1) that not only possess nanomolar affinity but also efficiently inhibits tau protein aggregation with higher inhibitory effects than the previously reported tau aptamers. Meanwhile, we further demonstrated that the truncated and modified aptamer (BW1c; 57 nucleotides) exhibited better affinity and higher inhibiting ability against hyperphosphorylation and aggregation of tau in vitro. BW1c also have and robust anti-Tau hyperphosphorylation/aggregation activities when introduced into neural cell culture. It is believed that this novel class of Tau aptamers serve as therapeutic agents in mitigating tauopathy-associated neurodegenerative disorders. This “Functional SELEX” approach demonstrated here establishes a new framework for discovering aptamers that not only based the selection/enrichment on target-recognition, but on target-specific biofunction interference.

### Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

The foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise.

As used herein, the term “about,” means plus or minus 20 percent of the recited value, so that, for example, “about 0.125” means 0.125±0.025, and “about 1.0” means 1.0±0.2.

As used herein, the term “linked” refers to a covalent linkage.

As used herein, the term “analog” refers to a compound with similar properties.

As used herein, the term “animal” refers to humans as well as non-human animals. Non-human animals preferably are mammals (e.g., laboratory animals, companion animals, farm animals, and the like, including, rats, mice, rabbits, monkeys, primates, dogs, cats, cattle, sheep, goats, swine and the like) and may be a transgenic animal.

As used herein, the term “antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes Fab′ and F(ab)′2 fragments. The term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. The “Fc” portion of an antibody refers to that portion of an immunoglobulin heavy chain that comprises one or more heavy chain constant region domains, CH1, CH2 and CH3, but does not include the heavy chain variable region.

As used herein, the terms “aptamer” refer to an oligonucleotide molecule that binds to a target protein. Aptamers can be comprised of RNA or DNA. Aptamer embodiments are described herein typically as DNA based aptamers. It will be understood that the DNA aptamer sequences described can be in the form of RNA sequences where any thymine sequences are presented as uracil. In some embodiments of the invention, the aptamer aptamer binds to a specific region or amino acid sequence of the target protein. The term “Tau aptamer” refers to an aptamer that can binds to a Tau protein at a phosphorylatable site. A “TfR aptamer” is an aptamer that specifically binds to transferrin receptor (TfR). A “Tau (IT2a) aptamer” is an aptamer that specifically binds to Tau. A circular Tau-TfR aptamer is a bifunctional aptamer that specifically binds both to Tau and to TfR. A trifunctional aptamer is a Y-shaped structure containing three aptamer moieties that each specifically bind a protein, for example Tau (microtubule binding protein Tau), TfR (Transferin receotpr incuding transferrin receipt 1 and 2) and L1CAM (L1 cell adhesion molecule).

As used herein, the terms “bind,” “binding,” and their cognates refer to any type of chemical or physical binding, which includes but is not limited to covalent binding, hydrogen binding, electrostatic binding, biological tethers, transmembrane attachment, cell surface attachment and expression. The terms “specifically bind,” “specific binding,” and their cognates refers to an adherence of one molecule for another which reflects a complementarity between them, for example a ligand and receptor, an enzyme and substrate, an antibody and antigen or hapten, aptamer and target, or two complementary nucleotide strands.

As used herein, the term “conjugate” refers to connected, coupled, or linked molecules. In particular, a DNA aptamer conjugate refers to a DNA aptamer connected, coupled, or linked to another molecule such as a reporter molecule, or a signaling moiety.

As used herein, the term “conservatively modified variants,” with respect to nucleic acid sequences, refers to those nucleic acids that encode identical or conservatively modified variants of the encoded amino acid sequences. Because of the degeneracy of the genetic code, a large number of functionally identical nucleic acids encode any given protein. With respect to amino acid sequences, the term refers to sequences of amino acids that contain substitutions of the traditional amino acids only within the same class, i.e., acidic, basic, aromatic, hydrophobic, hydrophilic.

As used herein, the term “diagnostic” refers to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, the term “diagnostic amount” of a marker refers to an amount of a marker in a subject's sample that is consistent with a diagnosis of neural injury and/or neuronal disorder. A diagnostic amount can be either in absolute amount (e.g., μg/mL) or a relative amount (e.g., relative intensity of signals compared to control).

As used herein, the term “dosage form,” refers to the particular dosage of a pharmaceutical product prepared in individual doses for administration. Dosage forms typically involve a mixture of active drug components and nondrug carrier components (also known as excipients), and optionally including containers or packaging.

As used herein, the term “dosage” refers to a specific amount, number, and frequency of administrated portions of active compound over a specified period of time. It can include one administration or a course of administrations. A “dosage regimen” is a treatment plan for administering doses of a drug over a period of time and also can include one administration or a course of administrations. The term “dose” refers to a specified amount of medication or active agent taken at one time.

As used herein, the term “drug” refers to a material that may have a biological effect on a cell, including but not limited to small organic molecules, inorganic compounds, polymers such as nucleic acids, peptides, saccharides, or other biologic materials, nanoparticles, etc.

As used herein, the term “effective amount” refers to an amount of an agent sufficient to exhibit one or more desired effects when administered in one dose, a course of doses or over a dosage regimen. The effective amount may be determined by a person skilled in the art using the guidance provided herein.

As used herein, the term “gene expression” refers to a process by which information from a gene is used to synthesize of a functional gene product. A gene product is often a protein, but in a non-protein coding gene such as transfer RNA (tRNA) or small nuclear RNA (snRNA) gene, the product is a functional RNA.

As used herein, the term “gene therapy” refers to the purposeful delivery of genetic material to cells for the purpose of treating disease or biomedical investigation and research. Gene therapy includes the delivery of a polynucleotide to a cell to express an exogenous nucleotide sequence, to inhibit, eliminate, augment, or alter expression of an endogenous nucleotide sequence, or to produce a specific physiological characteristic not naturally associated with the cell. In some cases, the polynucleotide itself, when delivered to a cell, can alter expression of a gene in the cell.

As used herein, the term “in need” in the context of a subject or patient refers to a subject that is in need of diagnosis or treatment for a disease or condition related to tauopathy or the presence of P-Tau or Tau aggregations or tangles. Such a subject may suffer from a tauopathy or be suspected of suffering from a tauopathy.

As used herein, the term “neural cells” refers to the cells that reside in the brain and central nervous system, or in the peripheral nervous systems, and include but are not limited to nerve cells, glial cells, oligodendrocytes, microglia cells or neural stem cells.

As used herein, the term “nucleic acid” and the term “polynucleotide,” as used interchangeably herein, refer to a deoxyribonucleotide or ribonucleotide polymer in either single- or double-stranded form, and unless otherwise limited, encompasses known analogs having the essential nature of natural nucleotides in that they hybridize to single-stranded nucleic acids in a manner similar to naturally occurring nucleotides (e.g., peptide nucleic acids).

As used herein, the term “oligonucleotide,” the term “polynucleotide,” the term “nucleotide,” and the term “nucleic acid” refer to a molecule comprised of two or more deoxyribonucleotides or ribonucleotides, and usually more than ten. The exact size of an oligonucleotide will depend on many factors, which in turn depends on the ultimate function or use of the oligonucleotide. The oligonucleotide may be generated in any manner, including chemical synthesis, DNA replication, reverse transcription, or a combination thereof. When present in a DNA form, the oligonucleotide may be single-stranded (i.e., the sense strand) or double-stranded.

As used herein, the term “percentage of sequence identity” refers to the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the term “pharmaceutical composition” as part of the disclosed invention, refers to a product comprising one or more of the disclosed Tau protein-binding DNA aptamers, and an optional carrier comprising inert ingredient(s), as well as any product that results, directly or indirectly, from combination, complexation or aggregation of any two or more of the ingredients, or from dissociation of one or more of the ingredients, or from other types of reactions or interactions of one or more of the ingredients.

As used herein, the term “pharmaceutical formulation” refers to a composition containing an active agent and a carrier mixture, combined to produce a medicinal product, such as a sterile solution, a capsule, a tablet, a powder, a granule, a solution, an emulsion, a gel, ointment, cream, lotion or the like for administration to a subject by any convenient method. A “pharmaceutical formulation” also includes delayed-release or sustained-release preparations. The medicinal product will vary by the route of administration which is to be used. For example, oral drugs are normally taken as tablet or capsules. Preferred pharmaceutical formulations for embodiments of the invention include forms of injection such as intravenous, intraarterial, and intraventricular or intracranial injection or infusion into the brain or CSF.

As used herein, the term “pharmaceutically acceptable” refers to a compound or group of compounds that are nontoxic and compatible with the other ingredients of the formulation and not deleterious to the subject.

As used herein, the term “pharmaceutically acceptable salt” refers to those salts that are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well-known in the art. Pharmaceutically acceptable salts may be obtained using standard procedures well known in the art, for example by mixing a compound of the present invention with a suitable acid, for instance an inorganic acid or an organic acid.

As used herein, the term “pharmaceutically acceptable carrier” refers to any carrier(s), used to facilitate administration of a pharmaceutical compound, that do not induce the production of antibodies harmful to an individual or a subject receiving a composition, is nontoxic and non-reactive, and is chemically and biologically compatible with the active agent. For example, pharmaceutically acceptable carriers may be large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Therefore, the term “pharmaceutically acceptable carrier” refers to nontoxic and nonreactive compound or agent that facilitates the incorporation of an active agent or pharmaceutical compound to form a pharmaceutical composition for administration to an animal.

As used herein, the term “polynucleotide” refers to a deoxyribopolynucleotide, ribopolynucleotide, or analogs thereof that hybridize, under stringent hybridization conditions, to substantially the same nucleotide sequence as the naturally occurring nucleotides and/or allow translation into the same amino acid(s) as the naturally occurring nucleotide(s). A polynucleotide can be full-length or a partial sequence, single- or double-stranded. Unless otherwise indicated, the term refers to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two non-limiting examples, are polynucleotides as the term is used herein. A great variety of modifications have been made to DNA and RNA that serve many useful purposes known to those of skill in the art. The term polynucleotide as it is employed herein encompasses such chemically, enzymatically or metabolically modified forms of polynucleotides, as well as the chemical forms of DNA and RNA characteristic of viruses and cells, including inter alia, simple and complex cells.

As used herein, the term “polypeptide” and the term “protein” are used interchangeably herein to refer to a polymer of amino acid residues. The terms encompass amino acid polymers in which one or more amino acid residues are artificial chemical mimetic of a corresponding naturally occurring amino acids, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” include glycoproteins, as well as non-glycoproteins.

As used herein, the term “recombinant” refers to a genetic material formed by a genetic recombination process. A “recombinant protein” is made through genetic engineering. A recombinant protein is encoded by a recombinant nucleic acid sequence that has a sequence from two or more sources incorporated into a single molecule.

As used herein, the term “specifically binds” and its cognates refers to binding to a specific binding partner and that can bind to and determine the presence of the binding partner in a heterogeneous population of proteins and other biologics without undue binding to other molecules in the sample. Thus, under designated conditions, the specific binder binds to its particular specific binding partner at least two times (and preferably more than 10-100 times) the background and does not bind substantially or in a significant amount to other molecules present in the sample. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

As used herein, the term “subject” or “patient” refers to an animal or a human individual, which is the object of treatment, diagnosis, observation or experiment.

As used herein, the term “Tau” refers to a microtubule-associated protein expressed abundantly in the neurons of the central nervous system. Its main function is to modulate the stability of axonal microtubules. The accumulation of hyperphosphorylated Tau in neurons leads to neurofibrillary degeneration, and various neurological symptoms. “P-Tau” refers to hyperphosphorylated or phosphorylated (pathological) Tau.

As used herein, the terms “tauopathy” or “tauopathy-associated disorder”” refer to a class of neurodegenerative diseases involving the aggregation of Tau protein into neurofibrillary or gliofibribrillary tangles, formed when hyperphosphorylation of Tau causes the protein to dissociate from the microtubule and form insoluble congregates or tangles. Tauopathies include AD, primary age-related tauopathy, chronic traumatic encephalopathy, progressive supranuclear palsy, corticobasal degeneration, frontotemperal dementia and Parkinsonism linked to chromosome 17 (FTDPL17), primary age-related tauopathy (PART). Lytigo-bodig disease, ganglioglioma, gangliocytoma, meningioangiomatosis, postencephalic parkinsonism, subacute sclerosing panencephalitis, and others.

As used herein, the term “therapeutically effective amount” refers to an amount of a compound or composition that, when administered to a subject for treating a disease or disorder, or at least one of the clinical symptoms of a disease or disorder, is sufficient to affect such disease, disorder, or symptom. This amount may be administered in a single dose or in a regimen of doses that may continue for a finite period or for continue for the life of the subject. A “therapeutically effective amount” will vary depending on the compound to be administered, the disease, disorder, and/or symptoms to be treated, severity of the disease, disorder, and/or symptoms, and the condition of the subject (including gender, age, weight, and health of the subject to be treated, and the general condition of the subject according to the treating doctor's assessment of the medical situation, and other relevant factors). An appropriate amount in any given instance may be readily ascertained by those skilled in the art or capable of determination by routine experimentation. It is expected that the amount will fall in a relatively broad range that can be determined through routine trials.

As used herein, the term “treating” and the term “treatment,” when being used in relation to a disease, disorder, or condition, refers to an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

### Overview

The invention disclosed here shows that wildtype and hTau mice subjected to repetitive closed head injury results in increased tauopathy and can serve as models for CTE. To tackle the unmet need to treat CTE and other tauopathy diseases, the work discussed here involves SELEX (Systematic Evolution of Ligands by EXponential enrichment) aptamer screening, used to discover a series of high affinity site-specific Tau-binding DNA aptamers. These Tau aptamers can partially inhibit Tau hyperphosphorylation and Tau aggregation. In addition, further modifications have improved the properties of the most promising Tau aptamer candidate (IT-2a) in two important ways: (i) aptamer circularization to improve plasma stability; and (ii) construction of bifunctional Tau/Transferrin receptor 1 (TfR)-binding aptamer to improve its ability to permeate the blood-brain barrier (BBB).

The invention comprises four new designs for aptamers:

(1) A bifunctional aptamer that can concurrently bind to Tau and TfR, to allow for transferrin receptor-based transcytosis of the bifunctional aptamer through the vascular endothelial cell layer, then facilitating crossing the BBB, and allow the bifunctional aptamer to reach the brain tissue where it can engage the extracellular and intracellular Tau target.

(2) A trifunctional aptamer that can concurrently bind to Tau, TfR, and neuronal cell adhesion L1CAM to facilitate penetrating the BBB as well as facilitate aptamer internalization into neurons and other brain cell types for both extracellular and intracellular Tau target engagement.

(3) An augmented Tau aptamer (IT2a) comprising a protein aggregate binding moiety (e.g. methylene blue) that bind to Tau monomer with high affinity and strong kinetics to prevent Tau oligomerization and aggregation in vitro. Methylene blue (MB) is a good model molecule for Tau aggregate binding and disruption, yet it has tendency to bind to hydrophobic pockets in other proteins thus it has off-target effects and cytotoxicity. By linking the Tau T231-epitope-specific aptamer IT2a to MB, which binds to tubulin repeats, a chemically augmented Tau aptamer that maximize Tau aggregation disruption can be produced.

(4) Coupling Tau/TfR1 circular aptamers or Y-shape Tau-TfR-L1CAM aptamers to (Gd3+) chelator “cage” DOTA-moiety can assist selective MRI imaging of Tau protein or aggregations. Many MRI-visible compounds (e.g., DOTA-Gd3+) do not penetrate the brain spontaneously and usually require the use of invasive techniques (e.g., BBB transient opening using hyperosmotic agents or ultrasound-associated microbubble injections) to reach their target. Gadolinium (Gd3+) contrast magnetic resonance based imaging (MRI) could not cross the intact BBB with no selectivity to Tau.

### Summary of Results

After subjecting human Tau-overexpressing (hTau) mice to the controlled cortical impact surgery, five daily doses of circular Tau-TfR aptamer leads to the attenuation of Tau and P-Tau levels in the cortex and hippocampus. Furthermore, treating hTau mice with circular Tau-TfR aptamer abrogates fluid percussion injury-induced spatial memory deficits. Therefore, this novel circular Tau-TfR bifunctional aptamer displays significantly improved plasma stability, brain exposure, as well as the ability to disrupt tauopathy and improve traumatic brain injury (TBI)-induced cognitive/memory deficits in vivo, providing important evidence that circular Tau-TfR aptamer can serve as a diagnostic and therapeutic compound for tauopathies.

An aptamer compound linked to a Gd⁺³ moiety is able to cross the BBB and target Tau for MRI imaging of Tau aggregates in vivo in the brain.

### DETAILED DESCRIPTION OF EMBODIMENTS

### Nucleic Acid Aptamers

Nucleic acid aptamers are short, single-stranded DNA or RNA oligonucleotides capable of specific binding to defined targets. The term “aptamer” derived from the Latin “aptus,” meaning to fit, was firstly introduced back in 1990 when Ellington and Szostak reported the in vitro selection of dye-binding RNA aptamers. The amplification-evolution process used to select these binding molecules was termed “systematic evolution of ligands by exponential enrichment” (SELEX) by Turek and Gold when they identified two RNA sequences that bind to T4 DNA polymerase from over 60 thousand species using this procedure.

Since then, a growing number of RNA and DNA aptamers have been selected against a variety of targets, including metal ions, fluorescent dyes, amino acids, nucleotides, antibiotics, metabolites, peptides, proteins, viruses, organelles, or even whole cells. Aptamers have shown remarkable specificity in discriminating targets from their similar analogs, such as differentiating among homologous proteins that differed only by a few amino acids or one single amino acid, or even between enantiomers. As oligonucleotides, aptamers are readily synthesized and are reproducible by chemical synthesis. In addition, functional modules, such as fluorophores, chemical linkers, therapeutics, or even nanoparticles, can be introduces onto aptamers to fulfill specific needs. The specificity and high affinity of aptamers' binding ability toward corresponding targets have made them a new generation of molecular probes. Especially, aptamers have shown outstanding capacity in differentiating specific disease-related proteins, either on the cell membranes or in the body fluids.

The Tau aptamers identified here have been produced against peptide fragments from Tau that have a predisposition to phosphorylation. The Tau aptamers according to this invention not only recognize Tau at the designated sites, but also demonstrate inhibitory effects on phosphorylation and oligomer formation. These properties allow practitioners to apply the Tau aptamers to (1) detect the levels of Tau in cerebrospinal fluid and brain tissue, (2) study the mechanism of tauopathy, and (3) arrest the progression of tauopathy-associated disorders.

### Aptamer-Protein Interactions

Many proteins in nature, such as transcription factors and nuclear proteins, are already known to interact with DNA or RNA to perform multiple functions and regulate many cellular processes, including transcription, translation, gene silencing, microRNA biogenesis and telomere maintenance. Even though these interactions are not necessarily strong, they are specific and functional. Unlike these DNA- and RNA-binding proteins, the proteins targeted by aptamers, and used to develop aptamers are proteins that do not normally interact with nucleic acids but show high affinity to the specific DNA or RNA selected for the aptamer. In these cases, the affinity may be attributed to the topography on the protein surfaces along with the presence of H-bond donors and acceptors as well as the flexible phosphodiester backbone of the nucleic acid that allows folding into precise three-dimensional scaffolds, thus creating discrete regions for hydrophobic and electrostatic interactions, hydrogen bonding, van der Waals forces, and shape complementarity between aptamers and target proteins to assist the recognition.

An example of this type of interaction is that of thrombin binding to single-stranded DNA aptamers with a highly conserved region. Binding thrombin with aptamers can inhibit thrombin activity and decrease the rate of blood clotting. The three-dimensional structure of the aptamer and the thrombin-aptamer complex have been evaluated by NMR and X-ray crystallography and show that aptamers are not only expected to recognize the targets but also inhibit their down-stream functions.

### Tau Proteins and Neuron Neurodegenerative Diseases

Tau proteins are neuronal microtubule-associated proteins known to promote the assembly of microtubules and to maintain microtubular integrity, which is physiologically essential for axonal transport and morphogenesis. These proteins are mainly expressed in neurons of the central nervous system (CNS), but are also found in astrocytes and oligodendrocytes at a much lower level. Tau is found to be pathologically involved in several related disorders, termed tauopathies, in which aggregations of Tau proteins are deposited in brain neurons. Tauopathies include, but are not limited to traumatic brain injury (TBI), frontotemporal dementia (FTD), Parkinson's disease, Alzheimer's disease, progressive supranuclear palsy (PSP), and a progressive degenerative disease of the brain often found in athletes, military veterans, and others with a history of repetitive traumatic brain injury, called chronic traumatic encephalopathy (CTE).

Tau was first isolated and identified in 1975 as a heat stable protein essential for microtubule assembly. Tau proteins found in adult human CNS are a mixture of six isoforms, ranging from 352 to 441 amino acids in length. Microtubule-associated protein Tau gene (MAPT), the gene encoding human Tau, contains 16 exons. Exon 1 (E1), E4, E5, E7, E9, E11, E12 and E13 are constitutive, whereas the others are subject to alternative splicing. The six human brain Tau isoforms are generated through alternative splicing of E2, E3 and E10. The Tau variants are expressed from alternative splicing in exons E2, E3, and E10 of a single MAPT gene. Among the six isoforms, three of them have three binding domains (3R isoforms), while the other three with exon 10 inserts have four binding domains (4R isoforms). These conserved microtubule-binding domains are located in the C-terminal half of Tau, and 3R isoforms bind less tightly to microtubules than 4R isoforms. Even though the molecular mechanism or the relative abundance of each Tau isoform is ambiguous currently, the inability of commercially available ELISA kits to discriminate between the 6 isoforms demonstrates that there are common epitope sites for each corresponding antibody across all 6 variants.

### Exemplary Neurodegenerative Diseases Related to Tauopathy

Alzheimer's disease (AD) is a neurological disorder that has symptoms of memory loss and cognitive decline resulting from progressive degeneration of or loss of neurons in the brain. The exact causes of Alzheimer's disease are not yet completely understood. It is the most common form of dementia as well as the most prevalent tauopathy. Two major inclusions often sighted in its progression or postmortem are extracellular plaques and intracellular tangles.

Plaques, or beta-amyloid plaques, are clumps of beta-amyloid monomers that appear between the dying neurons and interfere with neuron-to-neuron signaling. Consequently, brain functions such as memory can be seriously impaired because the neurons in the brain cannot signal and relay information properly. Amyloid plaques have also been found around blood vessels in the brain, causing amyloid angiopathy, which weakens the walls of blood vessels and increases the risk of hemorrhage.

Tangles, or neurofibrillary tangles (NFTs) are abnormal clusters of Tau proteins found within the brain neurons. Healthy neurons are held together by their cytoskeleton, which is partly built with microtubules. Normal Tau proteins bind with microtubules and prevent the track-like microtubule structures from breaking apart, allowing nutrients and molecules to be transported along the cells. Tau proteins in AD, on the other hand, lose their affinity for microtubules due to an abnormally high degree of phosphorylation. The pathological P-Tau proteins self-assemble into paired helical filaments (PHFs), which later aggregate into insoluble neurofibrillary tangles. The transport system for neurons is disrupted along the axon or other process, causing nutrients and other essential supplies to no longer move along the cells as needed. Neurons with tangles and non-functioning microtubules thereby undergo apoptosis and eventually cell death.

Chronic traumatic encephalopathy (CTE) is a progressive degenerative disease associated with repetitive traumatic brain injury (TBI). It has been found most commonly in professional athletes participating in contact sports and military personnel who have been exposed to a blast or other trauma. In this condition, there is often a long period of latency, ranging from several years to several decades, between the incident(s) of TBI and the occurrence of the clinical symptoms of CTE. The initial symptoms of CTE include irritability, impulsivity, aggression, depression, short-term memory loss and heightened suicidality, but it may progress further into cognitive deficits and dementia. The pathology of CTE is characterized by the accumulation of P-Tau protein in neurons and astrocytes (tauopathy).

### Hyperphosphorylation of Tau

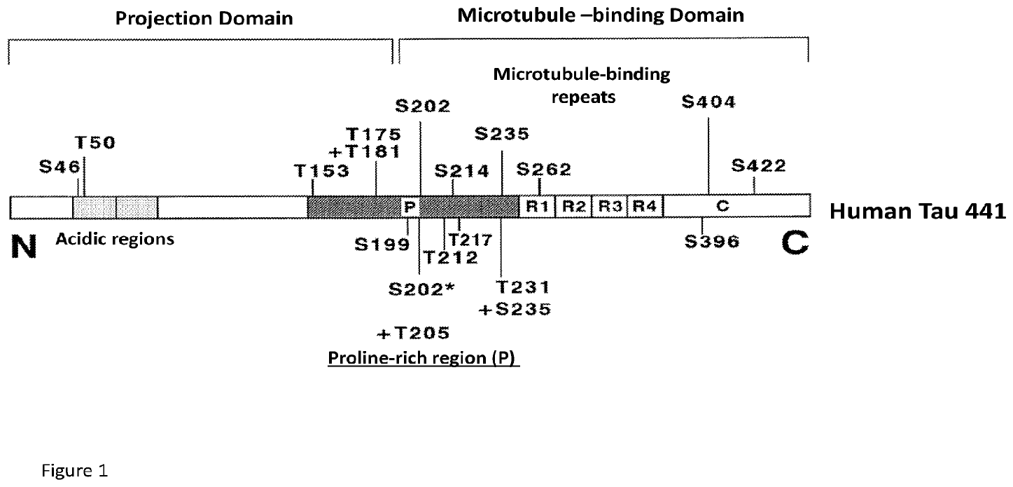

The distinctive intracellular neurofibrillary tangles observed in brains affected by Alzheimer's disease or chronic traumatic encephalopathy, or various forms of insoluble abnormal Tau aggregates in other tauopathies, share a common composition of pathological Tau, which is in an elevated state of phosphorylation, called hyperphosphorylation. Tau can be phosphorylated at many sites and by several kinases. A list of Tau phosphorylation sites identified is shown in FIG. 1. This figure shows the positioning of phosphorylation sites on Tau from Alzheimer brain.

Approximately 45 sites have been identified, and they seem to cluster in the PRD and in the C-terminal region, with few sites evident within the microtubule-binding domain of Tau. Six of the phosphorylation sites have been identified only by P-Tau-specific antibody labelling. The remaining phosphorylation sites have been identified by direct means (mass spectrometry and/or Edman degradation). Many of the reported phosphorylations occur at Ser-Pro and Thr-Pro motifs, corresponding to phosphorylated sites sensitized by proline-directed kinases (MAPK, GSK-3, cdk5), but the hyperphosphorylation of Tau is not confined within the proline-rich region. Apparently, increased phosphorylation in the microtubule-binding domain (residues 244-368) of Tau reduces the amount of Tau binding to microtubules. One site that has been reported to have a great impact on the binding of Tau to microtubules after its phosphorylation is Ser262. However, it is also found that phosphorylation of Tau at sites distinct from the microtubule-binding domain, such as at Thr231, still can have a pronounced influence on the binding affinity of Tau to microtubules. That being said, several other phosphorylation sites have only moderate effect on microtubule binding.

### DNA Aptamers Capable of Recognizing Disease-Associated Targets

According to embodiments of the disclosed invention, by using specific peptide fragments from Tau protein and pathologic P-Tau proteins as targets to carry out the selection for site-specific Tau protein-binding DNA aptamers (in short, “aptamers,” “Tau aptamers,” or “DNA aptamers”) that recognize Tau at designated phosphorylatable sites are identified and selected. Molecular probes are further developed using the site-specific Tau aptamers. Therefore, embodiments of the disclosed invention include DNA aptamers that not only recognize Tau at designated phosphorylatable sites, but also possess inhibitory effects on Tau phosphorylation and oligomer formation. These Tau protein-binding DNA aptamers are feasible to apply to (1) detect the levels of Tau and P-Tau in cerebrospinal fluid as capture or detection agent, (2) study the mechanism of tauopathy, and (3) arrest the progression of tauopathy associated neurodegenerative disorders.

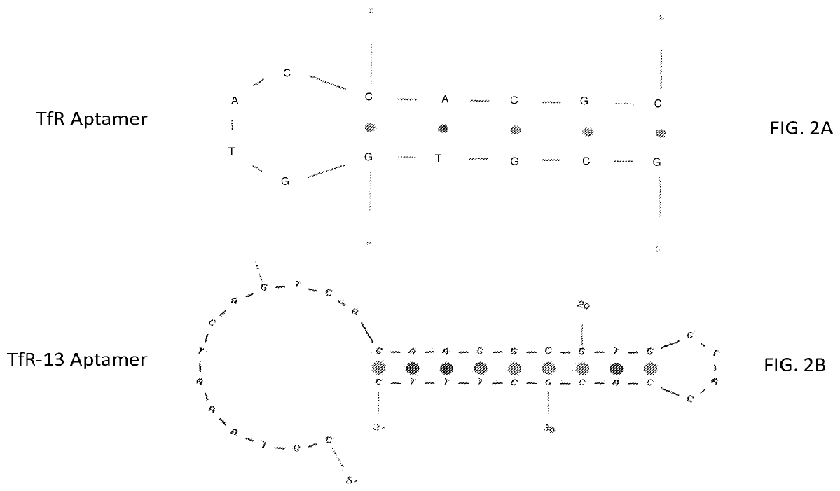

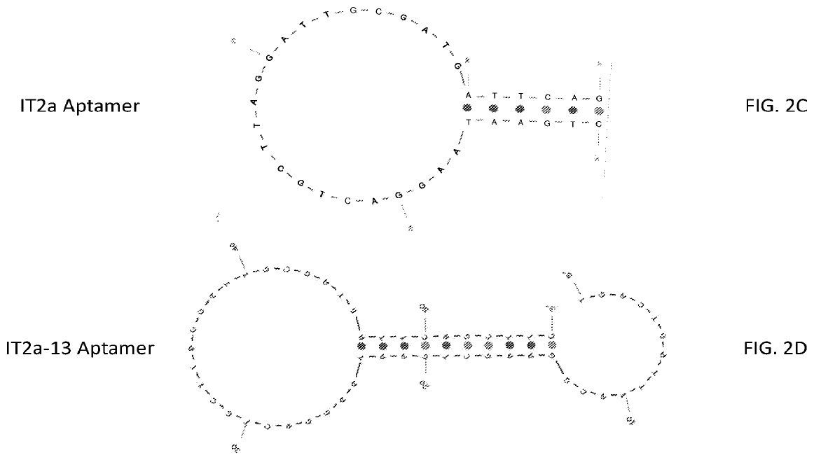

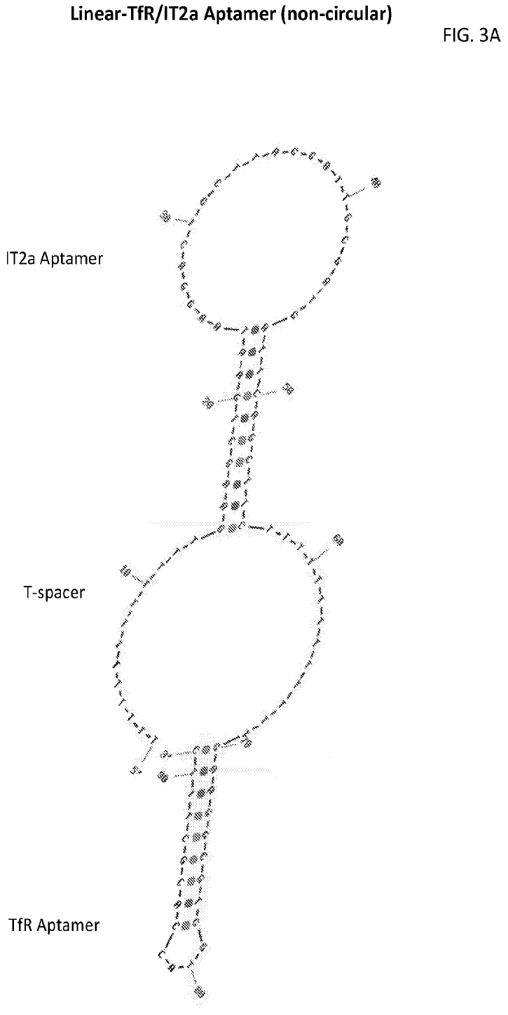

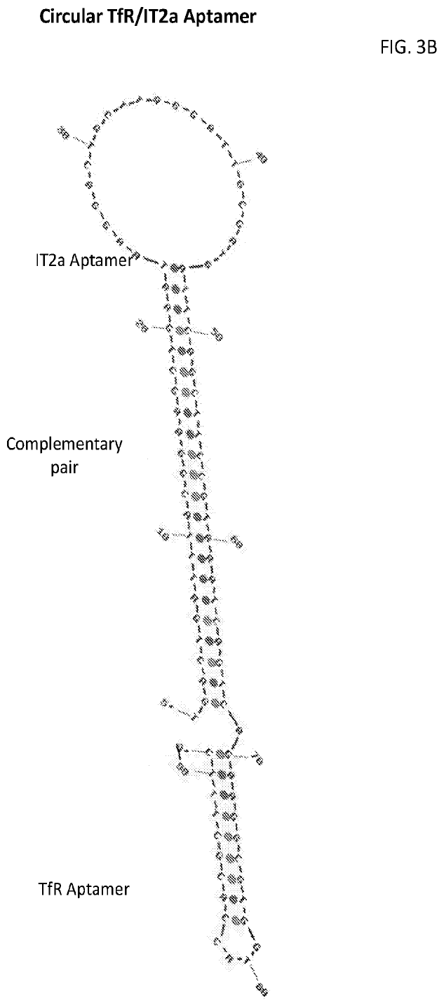

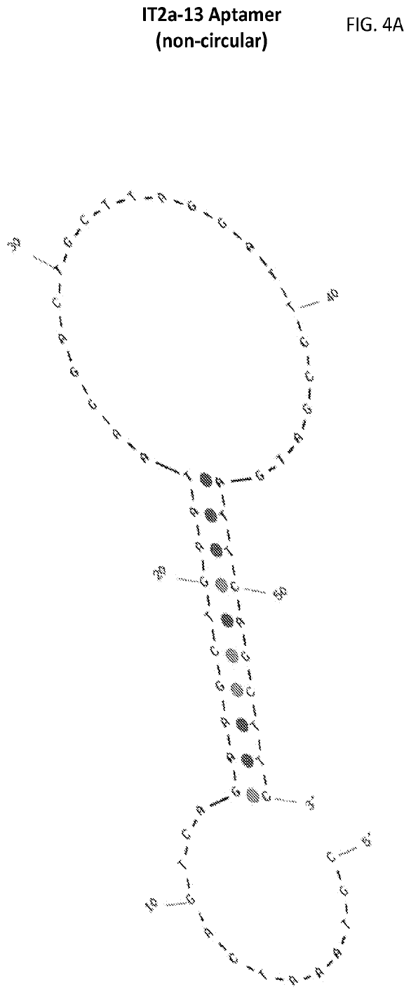

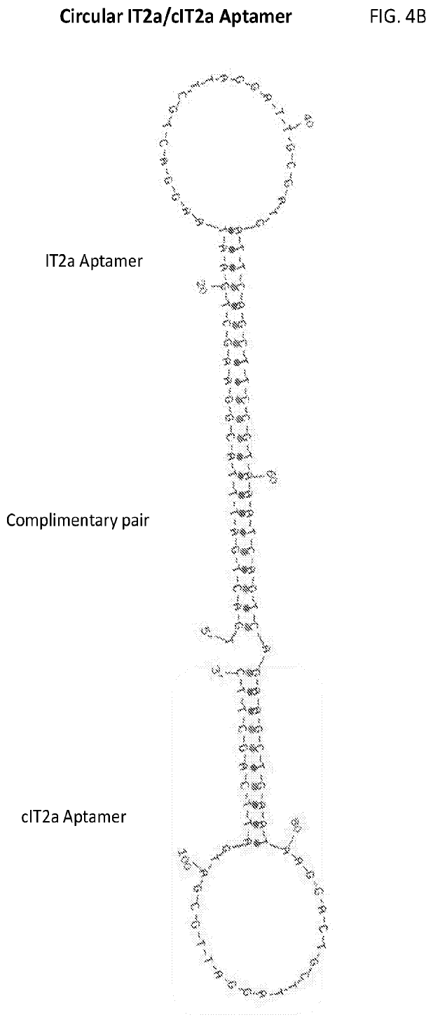

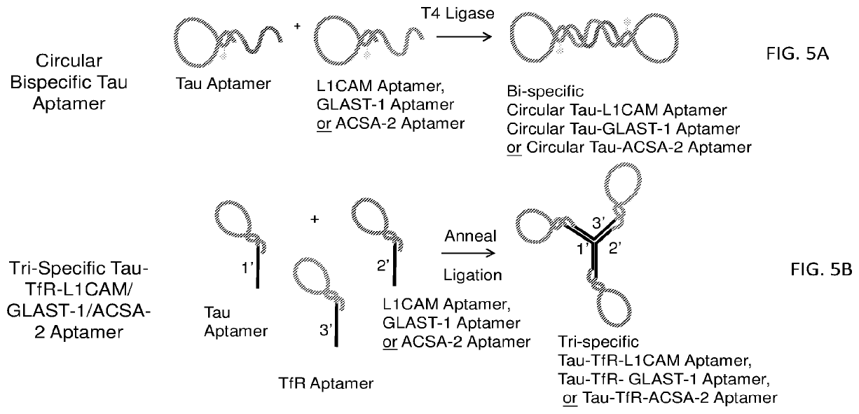

See FIG. 2, FIG. 3 and FIG. 4 for structures of certain aptamers as indicated. FIG. 5A depicts the concept of circular bispecific Tau Aptamers that can target two major cells type express tau protein and have been show to have tau aggregate despite, namely neurons and astrocytes. We use a novel concept of Circular Bispecific Tau Aptamer that fuse the tau-specific Aptamer sequence chemically to (i) a neuron cell surface antigen/adhesion molecule called L1CAM (also called CD171), and (ii) to astrocyte cell surface antigen (ACSA) including GLAST-1 (also called ACSA-1) and ACSA-2, respectively. The concept is that these Bispecific aptamers will then be internalized by endocytosis and thus can engage tau/tau oligomer/aggregate in their respective cells. Thus, these two synthesis will result in creating (i) circular Tau-L1CAM Aptamer to target and mitigate neuron tauopathy intracellularly. (ii) circular Tau-GLAST-1 Aptamer or circular Tar-ACSA-2 Aptamer will target and mitigate astrocyte tauopathy intracellularly.

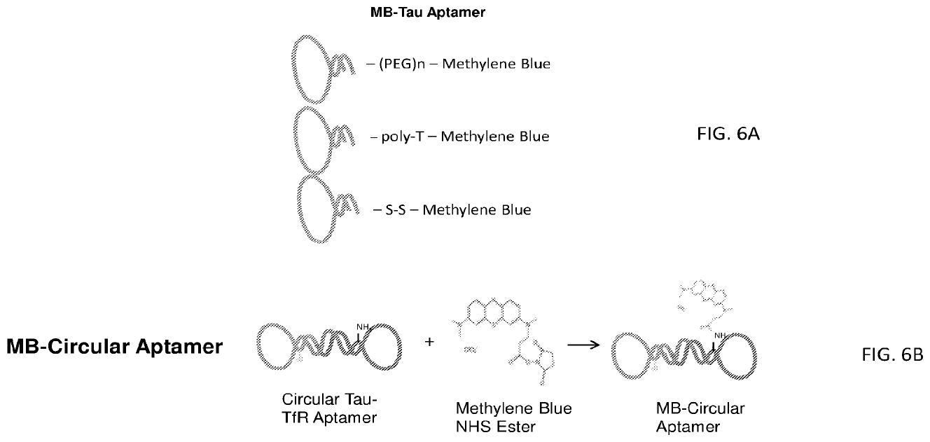

FIG. 5B depicts the concept that extend circular bispecific Tau Aptamer shown in FIG. 5A by constructing a tri-Specific Tau-TfR-L1CAM/GLAST-1/ACSA-2 Aptamer. The concept is that by fusing an TfR-binding aptamer to Tau aptamer and to L1CAM/GLAST-1/ACSA-2 Aptamers, respectively, this creates a y-shape tri-specific aptamer that (i) targets Tau for tauopathy mitigation, (ii) target L1CAM for neuron cell engagement and internalization or GLAST-1/ACSA-2 for astrocyte cell engagement and internalization, and then lastly (iii) targets TfR to facilitate brain vascular endothelial cell binding, and transcytosis to pass through the blood brain barrier (BBB). In reference to tri-specific aptamers that comprise a stem, such as 1′, 2′, or 3′ depicted in FIG. 5B, wherein the stem is an engineered sequence (typically 5 to 25 bp long), and wherein the respective stems possess sufficient complementarity to form a y-shaped configuration. See also FIG. 6A and FIG. 6B for additional structures for linking of methylene blue to the aptamers. To date, many other central nervous system (CNS)-related aptamers have also been reported as promising theragnostic probes specifically recognizing proteins that were relevant to neurodegenerative diseases, such as amyloid beta, myelin and alpha-synuclein. These aptamers usually have similar secondary hairpin structures and also showed efficient inhibitory effects on targeted protein aggregation.

### Pharmaceutical Compositions

In general, pharmaceutical compositions are prepared by uniformly and intimately bringing the active ingredient into association with a liquid carrier or a finely divided solid carrier or both, and then, if necessary, shaping the product into the desired formulation. A pharmaceutical composition includes enough of the active object compound to produce the desired effect upon the progress or condition of diseases. In certain embodiments, the aptamers described herein are formulated and are administered as pharmaceutical compositions that includes a pharmaceutically acceptable carrier and one or more of the inventive compounds described herein, or one or more of the inventive compounds described herein combined with an additional active agent. A pharmaceutically acceptable carrier refers to any convenient compound or group of compounds that is not toxic, non-allergenic, and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art. For example, a “pharmaceutically acceptable diluent, excipient, carrier, or adjuvant” is a diluent, excipient, carrier, or adjuvant which is physiologically acceptable to the subject while retaining the therapeutic properties of the pharmaceutical composition with which it is administered. One preferred pharmaceutically acceptable carrier is physiological saline.

A suitable carrier depends on the route of administration contemplated for the pharmaceutical composition. Routes of administration are determined by the person of skill according to convenience, the health and condition of the subject to be treated, and the location and stage of the condition to be treated. Such routes can be any route which the practitioner deems to be most effective or convenient using considerations such as the patient, the patient's general condition, and the specific condition to be treated. For example, routes of administration can include, but are not limited to: local, oral, or parenteral, including: oral, intravenous, intraarterial, intrathecal, subcutaneous, intradermal, intraperitoneal, rectal, vaginal, topical, nasal, local injection, buccal, transdermal, sublingual, inhalation, transmucosal, wound covering, direct injection into a specific area affected by disease, and the like. The administration can be given by transfusion or infusion, and can be administered by an implant, an implanted pump, or an external pump, or any device known in the art.

Therefore, the forms which the pharmaceutical composition can take will include, but are not limited to: tablets, capsules, caplets, lozenges, dragees, pills, granules, oral solutions, powders for dilution, powders for inhalation, vapors, gases, sterile solutions or other liquids for injection or infusion, transdermal patches, buccal patches, inserts and implants, rectal suppositories, vaginal suppositories, creams, lotions, oils, ointments, topical coverings (e.g., wound coverings and bandages), suspensions, emulsions, lipid vesicles, and the like.

Treatment regimens include a single administration or a course of administrations lasting one, two, or more days, including a week, two weeks, several weeks, a month, two months, several months, a year, or more, including administration for the remainder of the subject's life. The regimen can include multiple doses per day, one dose per day or per week, for example, or a long infusion administration lasting for an hour, multiple hours, a full day, or longer.

Dosage amounts per administration include any amount determined by the practitioner, and will depend on the size of the subject to be treated, the state of the health of the subject, the route of administration, the condition to be treated or prevented, and the like. In general, it is contemplated that for the majority of subjects, a dose in the range of about 0.01 mg/kg to about 100 mg/kg is suitable, preferably about 0.1 mg/kg to about 50 mg/kg, more preferably about 0.1 mg/kg to about 10 mg/kg, and most preferably about 0.2 mg/kg to about 5 mg/kg are useful. This dose can be administered preferably weekly, daily, or multiple times per day. A dose of about 0.01 mg, 0.1 mg, 0.2 mg, 0.25 mg, 0.5 mg, 1 mg, 5 mg, 10 mg, 20 mg, 40 mg, 80 mg, 100 mg, 250 mg, 500 mg, or 1000 mg can be administered.

### Methods of Use

The invention further includes methods for diagnosis and treatment of tauopathy-related diseases and conditions. Any tauopathy-related disease or condition is contemplated for use in the invention. Such diseases and conditions include, but are not limited to traumatic brain injury (TBI), frontotemporal dementia (FTD), and primary age-related tauopathy (PART), Parkinson's disease, Alzheimer's disease, progressive supranuclear palsy (PSP), and chronic traumatic encephalopathy (CTE).

Diagnostic methods contemplated as part of the invention include a neuroimaging test including MRI, near-infrared fluorescence based imaging diagnostic test or SPECT and PET imaging test.

Treatment methods contemplated as part of the invention include single or repeated treatment over time by various forms of administration including but not limited to intravenous, intraarterial, and intraventricular or intracranial injection or infusion.

### Some Advantages of the Disclosed Aptamers

Compared with the traditional small-molecule drugs and immunotherapy using anti-Tau antibodies, the Tau aptamer (IT2a) has demonstrated strong specificity and affinity against T-Tau protein, as well as inhibitory ability on Tau hyperphosphorylation and aggregation with low immunogenicity and cytotoxicity. To solve the BBB limitation problem, the Tau aptamer was circularized with a reported TfR aptamer that would facilitate the transport of the construct through TfR protein-mediated transcytosis on the BBB cell membrane.

The results discussed here show that the circular Tau-TfR bifunctional aptamer preferentially targeted specific Tau epitopes and T-Tau protein and was able to penetrate the BBB efficiently in both in vitro and in vivo models. Its potential for treating tauopathy disorders was also revealed by the effective reduction of related biomarker levels in TBI-induced traumatic brain tissues and the protective effects on impaired memory restoration. The circular aptamer of the invention also had better stability in body circulation.

Tauopathy attenuation may mainly result from the inhibition of circular Tau-TfR aptamer on extracellular Tau spreading and seeding among adjacent neurons; the aptamer showed low neuron cell membrane permeability. To overcome the neuron cell barrier and suppress the intracellular Tau aggregates, aptamers targeting neuron cell membrane proteins, such as anti-L1CAM aptamer, were included to make a trifunctional aptamer nanostructure that recognizes Tau, TfR, and L1CAM. In addition, introducing a Tau aggregation suppressor (e.g., methylene blue, thioflavin T, Pittsburgh compound B (PiB) (2-(4′-methylaminophenyl)-6-hydroxybenzothiazole), Riluzole, cyanine dye (Cy3), curcumin, catechin (a polyphenol), adriamycin Epalrestat and thionin, (Bruno Bulic, Marcus Pickhardt, Eva-Maria Mandelkow, Eckhard Mandelkow Tau protein and tau aggregation inhibitors Bruno Neuropharmacology 59 (2010) 276e289doi:10.1016/j.neuropharm.2010.01.016) onto the circular Tau-TfR aptamer via chemical modification can be used to strengthen the specificity and binding between drug and Tau aggregate, and to achieve better or synergistic effects for tauopathy treatment. It is also possible to modify circular aptamer with contrast agents to improve tauopathy-imaging diagnostics. Thus, by fabricating a circular Tau-TfR aptamer and a Tau-TfR-L1CAM aptamer with additional functional moieties, additional therapeutic and diagnostic compositions can be used to untangle, or even reverse neurodegenerative tauopathies.

### EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Exemplary Sequences

TABLE 1
Oligonucleotide Sequences.

| Name | | Sequence | SEQ ID NO |
|---|---|---|---|
| Circular | TfR Aptamer | 5′- [symbol] -CGTAAATCAGTCAGAAGGC | 1 |
| Aptamer | | GTGGTACCACGCTT(FITC)TC-3′ | |
| Tau-TfR | Tau Aptamer | 5′- [symbol] -TGACTGATTTACGGAAGCTGAATAAG | 2 |
| | (IT2a) | GACTGCTTAGGATTGCGAT GATTCAGC | |
| | | T(FITC)TC-3′ | |
| RS- | Scrambled | 5′- [symbol] -CGTAAATCAGTCAGAAGTCGACGCCG | 3 |
| Aptamer | TfR Aptamer | GTCGACT(FITC)TC-3′ | |
| Circular | Scrambled | 5- [symbol] -TGACTGATTTACGGAAGTTACGGACG | 4 |
| | Tau Aptamer | GATGTCAGTGGTATAGTAATCCGTAAC | |
| | (IT2a) | T(FITC)TC-3′ | |

| | | |
|---|---|---|
| Tau Aptamer (IT2a) for | 5′- [symbol] -TGACTGATTTACGGAAGCTGAATAAG | 5 |
| Cy5.5 Coupling | GACTGCTTAGGATTGCGATGATTCAGC | |
| | T(NH₂)TC-3′ | |
| cIT2a Aptamer | 5′- [symbol] -CGTAAATCAGTCAGAAGCTGAATAAG | 6 |
| | GACTGCTTAGGAT TGCGATGATTCAGC | |
| | T(FITC)TC-3′ | |
| Linear IT2a-TfR | 5′-CGTAAATCAGTCAGAAGGCGTGGTACC | 7 |
| Aptamer | ACGCTTTCTGACTGATTTACGGAAGCTGA | |
| | ATAAGGACTGCTTAGGATTGCGATGATTCA | |
| | GCT(FITC)TC-3′ | |
| Linear T-IT2a-TfR | 5′-TTTTTTTTTTTTTGAAGGCGTGGTACCAC | 8 |
| Aptamer | GCTTTCTTTTTTTTTTTTTGAAGCTGAATAA | |
| | GGACTGCTTAGGATTGCGATGATTCAGC | |
| | T(FITC) TC-3′ | |
| TfR Aptamer | 5′-GC GTG GTAC CAC GC-3′ | 9 |
| IT2 Aptamer | 5′-CAGCACCGTCAACTGAATAAGGACTGCT | 10 |
| | TAGGATTGCGATGATTCAGGGTGATGCGA | |
| | TTGAGATGT-3′ | |
| IT2a Aptamer | 5′-CTGAATAAGGACTGCTTAGGATTGCGA | 11 |
| | TGATTCAG-3′ | |
| TfR-13* Aptamer | 5′-CGTAAATCAGTCAGAAGGCGTGGTAC | 12 |
| | CACGCTTTC-3′ | |
| IT2a-13 Aptamer | 5′-TGACTGATTTACGGAAGCTGAATAAG | 13 |
| | GACTGCTTAGGATTGCGATGATTCAGCT | |
| | TC-3′ | |
| cIT2a-13 Aptamer | 5′-CGTAAATCAGTCAGAAGCTGAATAAG | 14 |
| | GACTGCTTAGGATTGCGATGATTCAGCT | |
| | TC-3′ | |
| Linear-TfR/IT2a | 5′-TTTTTTTTTTTTTGAAGCTGAATAAGGA | 15 |
| Aptamer (non-circular) | CTGCTTAGGATTGCGATGATTCAGCTTCT | |
| | TTTTTTTTTTTTGAAGGCGTGGTACCACG | |
| | CTTTC-3′ | |

*13 indicates that 13 extra bases were added to the aptamer sequence for the following annealing and ligation.

Circular TfR/IT2a Aptamer
(SEQ ID NO. 23)
5′-TGACTGATTTACGGAAGCTGAATAAGGACTGCTTAG
GATTGCGATGATTCAGCTTCCGTAAATCAGTCAGAAGGC
GTGGTACCACGCTTTC-3′
Circular IT2a/cIT2a Aptamer
(SEQ ID NO. 24)
5′-TGACTGATTTACGGAAGCTGAATAAGGACTGCTTAG
GATTGCGATGATTCAGCTTCCGTAAATCAGTCAGAAGCT
GAATAAGGACTGCTTAGGATTGCGATGATTCAGCTTC-3′

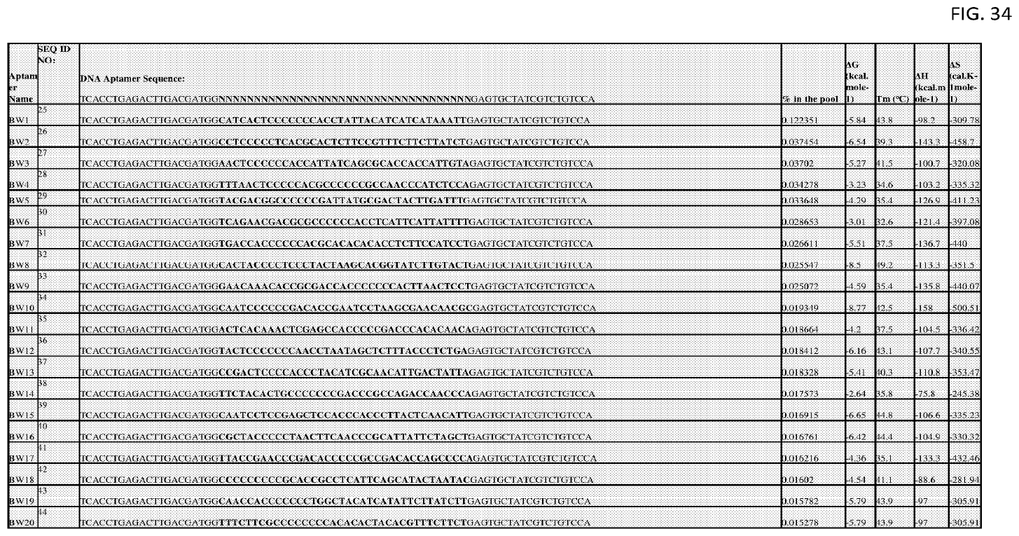

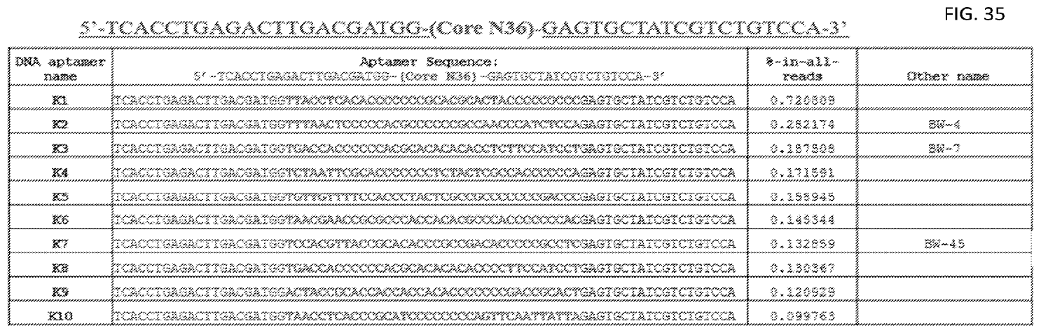

Other aptamer sequences are provided in the Figures such as FIGS. 34, 35 and 40, inter alia.

TABLE 2
Tau Peptides for Binding Studies of
Circular Tau-TfR Aptamer.

| Name | Sequence | SEQ |
|---|---|---|
| | | ID |
| | | NO |
| S202 | GYSSPGSPGTPGSR + linker − (His)₆ | 16 |
| T231 | KVAVVRTPPKSPS + linker − (His)₆ | 17 |
| T231P | KVAVVRT^{(p)}PPPKSPS + linker − | 18 |
| | (His)₆ | |

### Example 2: General Methods and Instruments

All oligonucleotide synthesis reagents were purchased from Glen Research Corp™ and ChemGenes™ Corp. The water used in all experiments was DNA grade from Fisher Scientific™. Dulbecco's phosphate buffered saline (PBS) with calcium chloride and magnesium chloride was purchased from Sigma-Aldrich™. Ultracentrifugation was performed by using Amicon® Ultra centrifugal filter units from Sigma-Aldrich™.

All peptides were purchased from GenScript™ with N-terminal acetylation and C-terminal 6 histidine residues (His-tag). Tau441 protein with N-terminal His-tag was purchased from SignalChem™. T4 DNA ligase, Exonuclease I and III (Exo I, III) were purchased from New England Biolabs™. Deoxyribonuclease I and mouse plasma (#50641960) were purchased from Sigma-Aldrich™ and Fisher Scientific™, respectively. Twelve millimeter collagen-coated transwell membrane inserts with 0.4 μm pore (#3493) were purchased from Corning™ Tetramethylrhodamine-labeled dextrans (#D1818: 70 k and #D1868: 10 k, MWCO) were purchased from Invitrogen™. All the gels (PAGE and agarose) were scanned using an Amersham BioSciences™ GE Typhoon 9410 Variable Mode Imager.

All oligonucleotides were synthesized on an ABI 3400 DNA synthesizer (Applied Biosystems™) using solid-state phosphoramidite chemistry. Synthesis started from controlled-pore glass (CPG) columns. Fluorescein-dT, amino-dT phosphoramidite and chemical phosphorylation reagent were directly coupled in the interchain or onto the 5′-end of oligonucleotides (Table 2). Sequences were deprotected in 2 mL APA solution (ammonium hydroxide:propylamine:water=2:1:1) at 65° C. for 1 hour. Deprotected sequences were purified by reversed-phase high-pressure liquid chromatography (HPLC) (ProStar™) using a C18 column. The collected sequences were then vacuum dried and quantified by measuring the absorbance at 260 nm using a Varian Cary™ 100 UV-Vis spectrometer (Agilent Technologies™).

Circular Tau-TfR aptamer was constructed using T4 DNA ligase and purified by urea-PAGE gel elution or ultracentrifugation. Flow cytometry and confocal microscopy were used to characterize the specificity of circular aptamer against Tau peptides/protein and TfR-positive bEnd.3 cells. An In vitro BBB model was established by culturing bEnd.3 cells on the transwell membrane inserts and was then used for transport efficiency analysis of circular aptamer. hTau mice were injected with certain amount of circular aptamer for the determinations of blood circulation time, biodistribution and ex vivo imaging. TBI animal model was developed by either performing CCI or FPI surgery for the assessment of therapeutic effects of circular aptamer on TBI-related biomarkers and memory deficits.

Both TfR aptamer and Tau aptamer (see Table 2) were dissolved in T4 DNA ligase buffer in 3 μM and heated for 10 minutes at 95° C. Then, 15 μL T4 DNA ligase were added into the solution after a quick chilling to 16° C. The reaction was carried out at 16° C. for at least 12 hours on a Multi-Therm™ shaker (Alkali Scientific™). Afterwards, the ligase in the solution was denatured by heating at 75° C. for 10 minutes. The constructed circular bifunctional aptamer was then extracted by using phenol-chloroform (#J75831-AN, Thermo Fisher Scientific™) and ethanol-salt precipitation. Finally, the dissolved DNA pellet was purified either by 8% urea-PAGE gel elution or 30 k/50 k MWCO ultracentrifugation. The concentration of purified Tau-TfR circular bifunctional aptamer was quantified by UV-Vis spectrometer.

To make Cy5.5-labeled circular aptamer, an amino-dT was first coupled in the interchain of Tau aptamer (see Table 2). After the ligation and purification of circular aptamer, as described above, a certain amount of 5 mM Cyanine5.5 NHS ester (abcam, #ab146455) in DMSO was added into 10 mg/mL circular aptamer solution in 1×PBS to make a concentration ratio of 1.2, and the reaction was carried out on a shaker at room temperature for 24 hours. Finally, 10 k MWCO ultracentrifugation was used to remove unbound Cy5.5 dye, and the concentration of Cy5.5-labeled circular aptamer for animal study was calculated based on Cy5.5 absorption at 632 nm.

For cell culture, HEK293T, bEnd.3 and SH-SY5Y cell lines were purchased from American Type Culture Collection (ATCC). HEK293T and SH-SY5Y cells were cultured in DMEM (Sigma-Aldrich™) with 10% fetal bovine serum (FBS, Gibco™) and 1% penicillin-streptomycin (PS, Life Technologies™), while bEnd3 cells were grown in DMEM (ATCC, 30-2002) supplemented with 10% FBS (ATCC, 30-2020) and 1% PS. Both cell lines were incubated at 37° C. in 5% CO₂.

Specificity analysis against Tau peptide and Tau protein was performed as follows. Selected Tau/p-Tau peptides were first immobilized on Ni Sepharose High Performance beads (GE Healthcare Life Sciences™) based on a previous method. Forty μL of stock Ni beads (2×10⁷ beads/mL) were washed with 1 mL PBS three times. After a quick spin-down, the supernatant was discarded. The pellet was resuspended in 100 μL of PBS containing 800 μg/mL of T231, T231P, S202 (Table 1) and incubated overnight at 4° C. on a shaker. Twenty μL of peptide-beads or bare beads (˜1.6×10⁵ beads) for each sample were washed three times with 500 μL of PBS. Then, each type of beads was incubated with 80 μL 200 nM of the TfR aptamer, Tau (IT2a) aptamer, and one FITC- or two FITC-labeled circular Tau-TfR aptamers for 30 minutes at 4° C. (1×PBS+5 mM Mg²⁺). Finally, beads were washed with 1 mL PBS five times and resuspended in 100 μL PBS for flow cytometry (BD Accuri™) analysis. The data was analyzed with FlowJo™ software.

To determine the specificity of the circular aptamer against Tau441 protein, 5 μL of 400 nM Tau (IT2a) aptamer or circular aptamer were first incubated with 0.2 mg/mL target Tau441 protein or control proteins (S100B, BSA) at 4° C. for 30 minutes. Then, each sample was loaded onto an 8% native PAGE gel. The gel was run in 1×TBE at 70 V for 10 minutes and 150 V for 40 minutes. Finally, the gel was stained with Coomassie blue for protein detection and scanned in the fluorescein channel using Typhoon for aptamer detection.

For specificity analysis against bEnd.3 cells, the methods were as follows. In the flow cytometry binding analysis, bEnd.3 and HEK293T cells were first detached using a nonenzymatic cell dissociation buffer and then incubated with 1× binding buffer at 37° C. for 30 minutes. Then, 1×10⁵ cells of both cell lines were counted and incubated with 200 μL 400 nM of each DNA sample in 1× binding buffer for 30 minutes at 4° C. Finally, the cells were washed with iced 1×PBS three times and suspended in 100 μL 1×PBS buffer for fluorescence analysis on a BD Accuri flow cytometer.

In the confocal microscopy studies, bEnd.3 and HEK293T cells (2×10⁴ cells/well) were 24 hours preloaded in a 4-chamber confocal dish. Then the cells were incubated with 400 nM 200 μL of two FITC-labeled circular aptamers at 37° C. in 5% CO₂ for 0.5 hour and 4 hours in opti-MEM® I medium (ThermoFisher Scientific™). Afterwards, the cells were incubated with a Hoechst™ stain for nuclear staining, followed by washing three times with iced 1×PBS. Finally, the fluorescence images were acquired using a Leica™ TCS SP5 confocal microscope with a 63× oil objective. The images were analyzed by using LAS AF.

In flow cytometry internalization tests, the same cell detachment procedure was performed as in the binding assay. Then, 1×10⁵ cells of bEnd.3 cells were incubated with 200 μL 400 nM of each DNA sample in opti-MEM® I medium for different time periods (0.5, 1, 4, 8 hours) at 37° C. in 5% CO₂. Afterwards, the cells were washed and incubated with trypsin for 10 minutes at 37° C. in 5% CO₂. Finally, the cells were washed with 1×PBS three times and were ready for flow cytometry analysis.

To determine the in vivo blood circulation time of mice, the following methods were used. Animal care conformed to the guidelines of the Institutional Animal Care and Use Committee (IACUC). C57/BL6 mice with an average weight of 22 g were selected to evaluate the blood circulation time of Cy5.5-labeled TfR aptamer, Tau aptamer and circular aptamer. Three mice in each group were anaesthetized using an isoflurane vaporizer and received retro-orbital injection of 150 μL 1×PBS containing each aptamer sample at a dose of 80 nmol/kg. At different time points (0.25, 0.5, 0.75, 1.5, 2, 4, 6, 12 and 24 hours) post-injection, 50-100 μL of blood were drawn from the tail vein. The blood samples were weighed and dissolved in 1×RBC lysis buffer (ThermoFisher Scientific™) to make 500 μL solutions for a 1-hour incubation with occasional shaking. Then 300 μL of each solution were collected for Cy5.5 fluorescence quantification in a 96-well black plate using a CLARIOstar™ plate reader. An additional 10 μL aliquot of each aptamer sample was saved and diluted for a total fluorescence measurement (Fₜₒₜₐₗ). Finally, mean injected dose per gram (% ID/g) in blood was calculated as % ID/g in blood=F_{blood}×V_{blood}/Fₜₒₜₐₗ/Vₛₐₘₚₗₑ/dilution factor/W_{blood}×100%.

Brain slice preparation and imaging was performed as followed. To further study the distribution of circular Tau-TfR aptamer in brain slices, C57/BL6 mice (25-30 g) were anaesthetized and perfused with 1×PBS followed by formalin after injection of 150 μL 700 nmol/kg of Cy5.5-labeled TfR aptamer, Tau aptamer and circular Tau-TfR aptamer each for 1 hour. The mice were sacrificed and brains were fixed overnight in formalin at 4° C., and were further dehydrated with 30% sucrose solution at 4° C. After embedding in O.C.T. (Sakura® Finetek™) for several days at −20° C., brains were processed for consecutive frozen sectioning with a thickness of 20 μm using a Microm™ Cryostat. The dissected brains were stained with DAPI for 10 minutes before imaging, and each entire brain slice image was captured by scanning the whole slide with z sections stacking using Leica TCS SP5 confocal microscope with a 20× dry objective.

TBI animal model and aptamer treatments were performed as follows. Traumatic brain injury (TBI) was induced by cortical control impact (CCI) surgery using the Leica Impact One™ system (Leica Biosystems™), according to previous literature. Tg(MAPT) 8cPdav/J (hTau) mice (Jackson Lab™, #005491, 4-6 months old, 25-30 g) were anaesthetized using an isoflurane vaporizer and maintained in deep plane of anesthesia during the surgery. A 1-2 cm midline cranial incision was made on the head, and a unilateral craniotomy (ipsilateral to the CCI site, ˜3 mm diameter) was performed adjacent to the central suture, midway between Bregma and lambda. The dura mater was kept intact over the cortex. Then TBI was induced on the right cortex using a Benchmark™ Stereotaxic Impactor with the impactor tip at a velocity of 3.5 m/s, compression depth of 1.5 mm and compression duration of 200 ms. After one week of recovery, mice with CCI were randomly assigned to three treatment groups: CCI only, CCI+circular aptamer, and CCI+Tau aptamer, each group containing 5-6 mice. After anesthesia, mice in the treatment groups received injection of 150 μL 200 nmol/kg of circular aptamer or Tau aptamer once a day for five days. During the study, mice were monitored closely for signs of infection, bleeding and distress.

Preparation of serum and tissue samples from the TBI model were obtained as follows. The serum samples were obtained through cardiac puncture after the mice were anaesthetized. The samples were subjected to centrifugation at 14,000 rpm for 30 minutes at 4° C. and the supernatant transferred to new Eppendorf™ tubes and stored at −80° C. for future analysis.

Mouse cortex, hippocampus and thalamus were collected from CCI mouse brains, snap-frozen in liquid nitrogen and stored at −80° C. for further analysis. The brain tissues then were homogenized using a cordless pellet pestle (Kimble™, #749540-0000) and were lysed for 2 hours at 4° C. using lysis buffer (5 mM EDTA, 1% Triton X-100, 1 mM DTT, and complete protease inhibitor cocktail (Roche™)). The supernatant was transferred to new Eppendorf™ tubes after 14,000 rpm centrifugation for 10 minutes at 4° C. The total protein concentration of each sample was determined using a Pierce™ 660 nm protein assay (ThermoFisher Scientific™) Each sample was also diluted to 500 ng/μL for future assessment.

Assessment of TBI-related biomarkers was performed as follows:

1. GFAP. Homebrew enzyme-linked immunosorbent assay (ELISA) was developed to evaluate the GFAP protein level in brain tissues. The rabbit anti-GFAP antibody (#29309) was first prepared in coating buffer (carbonate-bicarbonate buffer, sigma #3041) at 0.1 μg/mL and pipetted into a 96-well plate (100 μL per well) for overnight incubation at 4° C. Then the plate was washed three times with TBST (Tris Buffered Saline, with Tween® 20, pH 8.0, Sigma™) and incubated with 100 μL Start-Block™ blocking buffers (Thermo Scientific™, #37543) for 1 hour at room temperature on a shaker. After washing three times with TBST, human recombinant GFAP (Dx-SYS, #DXAG-001) in TBST as standard protein solutions with a series of concentrations were added to each well (100 μL), while 4 μL 500 ng/μL of brain tissue samples were mixed with 96 μL TBST and transferred to other wells. The plate was stored at 4° C. overnight. After washing, 100 μL 0.1 μg/mL of detecting mouse anti-GFAP (BD Pharmingen™ #556330) antibody in TBST was added into each well with incubation at room temperature for 1 hour. Afterwards, the plate was washed and treated with 100 μL 1:10K diluted anti-mouse IgG HRP (Jackson ImmunoResearch™) for 1 hour at room temperature. Finally, 100 μL TMB solution was added to each well to develop color for 20 minutes, then 100 μL stop solution was added, and the 450 nm and 620 nm absorbance was recorded by a plate reader. The final readings were determined by subtracting readings at 450 nm from readings at 620 nm, and the readings could then be converted to the concentration of each sample (ng/mL) according to the standard protein concentrations.

2. pNF-H. A commercial ELISA kit was used to analyze pNF-H level in brain tissues (BioVendor™, cat #RD191138300R). The assay was conducted based on the manufacturer's procedure.

3. Tau/P-Tau. The Tau and P-Tau levels in brain tissues were analyzed using MSD homebrew kit. Before ELISA, SULFO-Tag NNS was conjugated to polyclonal rabbit anti-Tau antibody (DAKO™, #A0024) as a detector according to company's manual and stored at 4° C. for future use. Firstly, 30 μL 0.5 μg/mL of capture antibody for Tau and P-Tau-DAKO™ and PHF-1 in 1×PBS was coated in homebrew plates (MSD, #L15XA-3) at 4° C. overnight. The next day, the plates were washed with TBST and blocked with 150 μL Start-Block blocking buffers for 1 hour at room temperature on a shaker. Then the standard protein calibrators were prepared by diluting Tau441 (rPeptide, #T-1001-1) or DYRK1A P-Tau (SignalChem™ #B1879-7) in TBST with serial dilutions. The calibrators (25 μL) or tissue samples (5 μL+20 μL TBST) were added into each well for incubation at 4° C. overnight. After washing, the plates were treated with 25 μL 1 μg/mL pre-prepared SULFO-Tag detector for a 1 hour incubation at room temperature. Finally, the plates were washed and incubated with 150 μL 1× read buffer and scanned by the MSD machine. The Tau levels in serum samples were also evaluated using the Simoa™ mouse Tau discovery kit (Quanterix™, #102209). The assay was performed following the manufacturer's procedure.

All data were expressed as the mean value with a standard deviation (±SD) or standard error (±SEM). Statistical analysis was performed using a t-test, and a P value <0.05 was considered significant. *P<0.05, **P<0.01, ***P<0.005.

### Example 3. Construction and Characterization of Circular Tau-TfR Bifunctional Aptamer

To make a simple and stable DNA nanostructure capable of both crossing the BBB and targeting Tau protein, one TfR aptamer and one Tau aptamer were first selected (see Table 2). The 14-nucleotide truncated TfR aptamer was short and easy to manipulate, while still keeping its selective recognition of TfR in vitro. The Tau aptamer used here was truncated (IT2a) with unique binding profile towards Tau at three important epitopes (Thr-231, Thr-231P and Ser-202; see Table 1) and has also demonstrated inhibitory effect on Tau oligomerization in vitro.

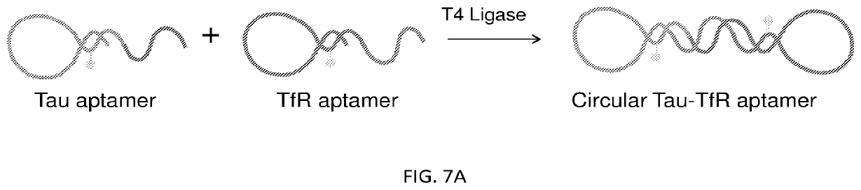

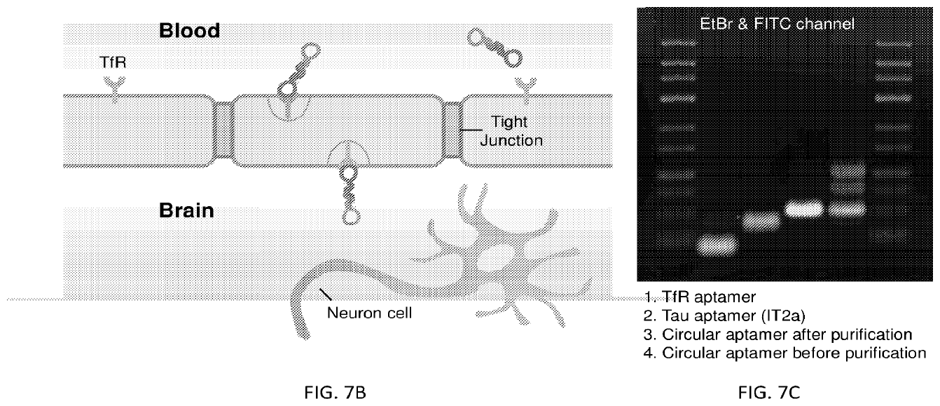

To maintain the loop structure for target recognition, the stems of two aptamers were elongated with several bases and inserted with one dT-FITC for fluorescence detection. Then the aptamers were modified with additional 13-base complementary sequences to allow hybridization and ligation for the formation of circular Tau-TfR bifunctional aptamer based on previous method. See FIG. 7A, which illustrates the creation of circular bifunctional aptamer incorporating transferrin receptor (TfR) aptamer and Tau protein aptamer IT2a via enzyme ligation by T4 ligase. The small moiety represents the FITC fluorescence tag. As illustrated in FIG. 7B, the generated circular Tau-TfR aptamer can be transported through BBB via TfR-mediated transcytosis, and help ameliorate tauopathy via Tau aptamer-facilitated reduction on several Tau-related pathological events. Once the circular bispecific aptamer enters the brain, it can potentially disrupt tauopathy at several points: (1) Tau hyperphosphorylation, (2) Tau oligomerization, and (3) Tau aggregation.

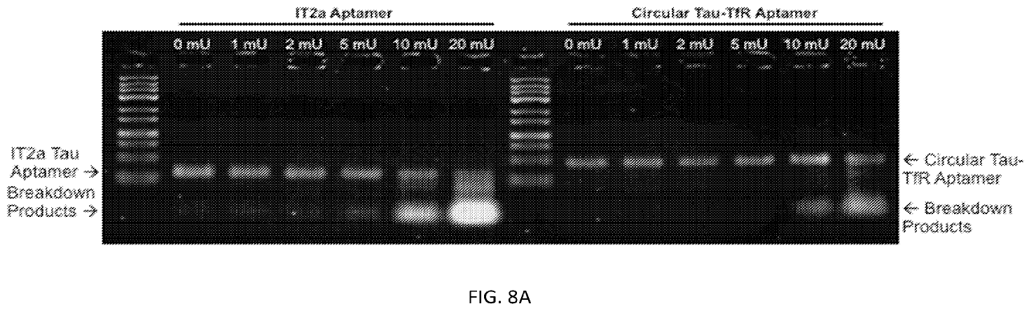

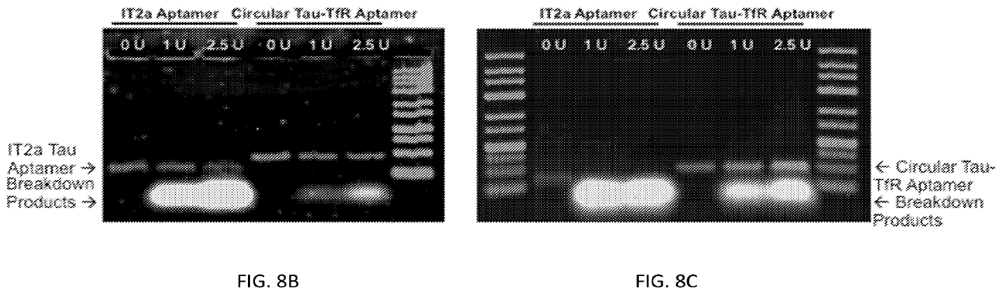

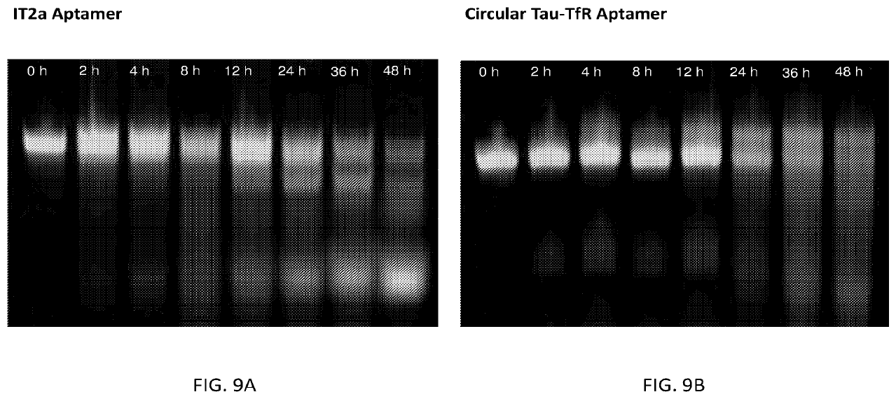

After purification, the successful construction of circular Tau-TfR aptamer was confirmed by agarose gel electrophoresis as the circular aptamer showed slower migration than its single-stranded constituents. The gel, shown in FIG. 7C, was scanned in both EtBr channel and fluorescein channel using Typhoon. Moreover, the biostability of circular Tau-TfR aptamer in different biological media was investigated. As there was no open nick, circular Tau-TfR aptamer exhibited remarkably improved resistance to nuclease degradation in vitro, compared with Tau aptamer alone (see FIG. 8 and FIG. 9). The circularization also showed impressive protection on the DNA structure against the more complex mouse plasma environment at physiological temperature, which was essential for the delivery of Tau aptamer in practical applications.

### Example 4. Aptamer Sample Stability Analysis

Stability analysis of aptamer samples was performed according to the following methods. In particular, for the DNase I and exonucleases resistance studies, Tau (IT2a) aptamer and circular aptamer (0.2 μM, 10 μL) were incubated with 1, 2, 5, 10 or 20 mU DNase I for 30 minutes at room temperature, or incubated with 1 U or 2.5 U Exo I or Exo III for 30 minutes at 37° C. Then, each sample was loaded onto 4% agarose gels. The gels were run in 1×TBE at 110 V for 40 minutes or 25 minutes and then scanned in both EtBr channel and fluorescein channel using Typhoon. See FIG. 8, which presents a set of 4% agarose gels showing the stability tests of circular IT2a/TfR aptamer versus Tau aptamer IT2a.

For FIG. 8A, Tau (IT2a) aptamer and circular aptamer were incubated with 1, 2, 5, 10 or 20 mU DNase I for 30 minutes at room temperature. For FIG. 8B, FITC-labelled IT2a and circular aptamers were incubated with 1 U or 2.5 U Exo I for 30 minutes at 37° C. For FIG. 8C, aptamers were incubated with 1 U or 2.5 U Exo III for 30 minutes at 37° C. The gels were scanned in both EtBr channel and fluorescein channel using Typhoon.

For the serum stability testing, Tau (IT2a) aptamer and circular aptamer (0.3 μM) were incubated with 10 μL DMEM cell culture medium containing 10% FBS at 37° C. for 0, 2, 4, 8, 12, 24, 36 or 48 hours. At the designated time point, each tube was taken out and heated at 95° C. for 5 minutes and then stored at −20° C. Then, 5 μL of each sample was loaded onto 12% urea PAGE gel. The gel was run in 1×TBE buffer at 70 V for 10 minutes and 120 V for 40 minutes, and the bands were captured using the fluorescein channel on Typhoon (FIG. 9).

### Example 5. Mouse Plasma and Brain Lysate Resistance Studies

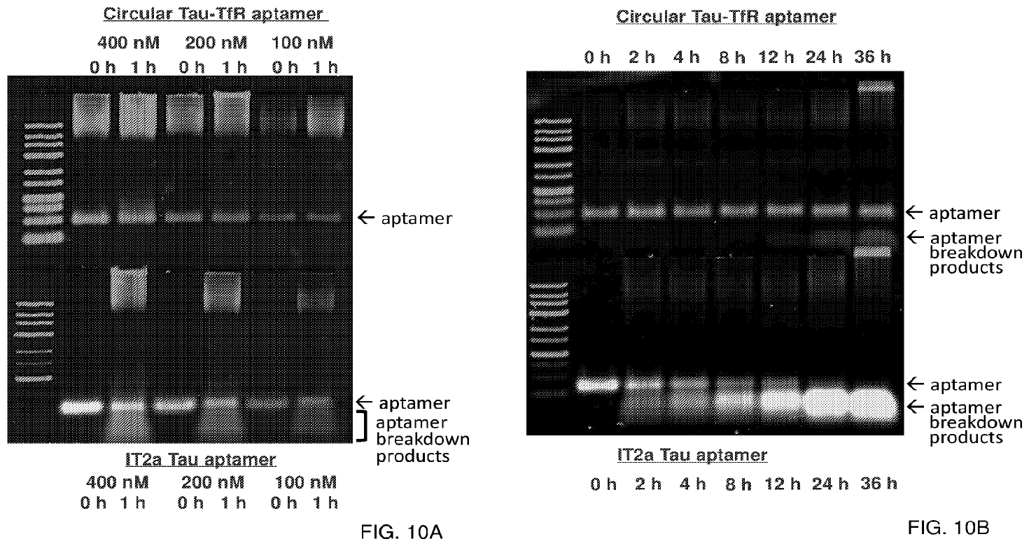

For mouse plasma and mouse brain lysates resistance studies, Tau (IT2a) aptamer and circular aptamer with different concentrations (400, 200, 100 nM, or 10 μL) were incubated in mouse plasma for 1 hour at 37° C., or samples (400 nM or 10 μL) were incubated in mouse plasma for different time (0, 2, 4, 8, 12, 24, or 36 hours) at 37° C., or samples (400 nM or 10 μL) were incubated in mouse brain lysates for different time (0, 2, 8, 12, or 24 hours) at 37° C. Then, each sample was loaded onto 4% agarose gels. The gels were run in 1×TBE at 110 V for 25 minutes and then scanned in both EtBr channel and fluorescein channel using Typhoon (FIG. 10). For FIG. 10A, IT2a and circular aptamer with different concentrations were incubated in mouse plasma for 1 h at 37° C. For FIG. 10B, 400 nM of IT2a and circular aptamer were incubated in mouse plasma for different time at 37° C. The gels were scanned in both EtBr channel and fluorescein channel using Typhoon.

### Example 6. Cell Viability Studies

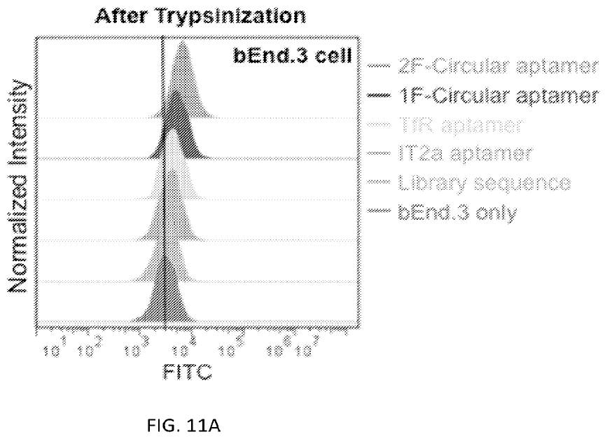

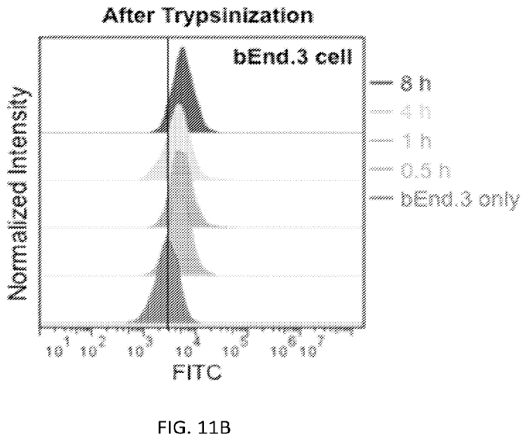

Cell viability studies were performed as follows. The cytotoxicity of TfR aptamer, Tau (IT2a) aptamer and circular aptamer on bEnd.3 cells and SH-SY5Y cells was evaluated by MTS assay (see FIG. 11). Cells (5×10⁴ cells/well) were preloaded in 24-well plates for 24 hours. Then, the cells were incubated with each sample at the desired concentration at 37° C. in 5% CO₂ for 8 hours. The supernatant was removed from each well, followed by washing with 1×PBS. Fresh full medium (500 μL) was added for 48 hours to allow additional cell growth. After removing the supernatant, MTS reagent (20 μL CellTiter™ reagent) diluted in fresh full medium (200 μL) was added to each well for 1 hour incubation. Finally, the 24-well plate was scanned by a CLARIOstar™ plate reader (BMG LabTech™), and the 490 nm absorbance was recorded and normalized to the signals from untreated cells.

For FIG. 11A, target bEnd.3 cells were incubated with 200 μL 400 nM different DNA samples at 37° C. for 1 hour. For FIG. 11B, target bEnd.3 cells were incubated with 200 μL 400 nM circular Tau-TfR aptamer at 37° C. for different times as indicated. The cells were incubated with trypsin to remove noninternalized aptamers on the cell surface before the flow test. FIG. 11 shows data regarding flow cytometry analysis which demonstrates the internalization ability of each DNA sample using cell trypsinization.

### Example 7. Circular Tau-TfR Aptamer Against Tau Protein Shows Specificity for Vascular Endothelial Cells—bEnd.3 Cells

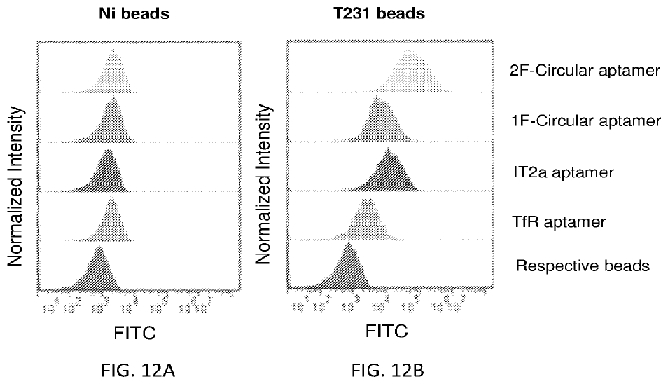

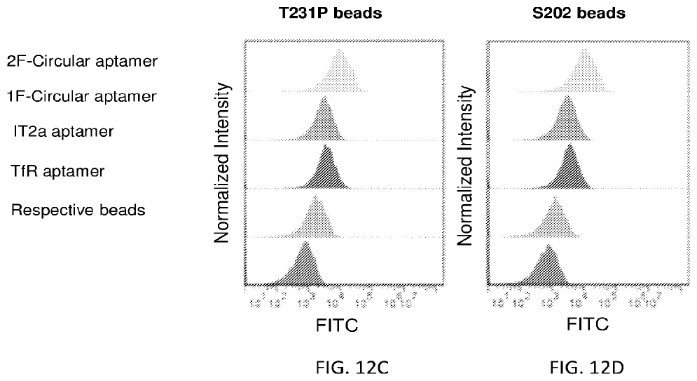

Next, we evaluated whether the circular Tau-TfR aptamer retained the specificity of both aptamers after circularization. FIG. 12A, FIG. 12B, FIG. 12C, and FIG. 12D provide flow cytometry histograms demonstrating the specific binding of circular aptamer against targeted Tau peptides. In these tests, 100 μL of 200 nM of TfR aptamer, Tau (IT2a) aptamer, and either one FITC- or two FITC-labeled circular aptamers were incubated with controlled Ni beads, T231-Ni beads, T231P-Ni beads or S202-Ni beads at 4° C. for 30 minutes. As examined by flow cytometry, IT2a itself showed good targeting ability for the three Tau peptides (immobilized on Ni beads). The one FITC-labeled circular Tau-TfR aptamer also displayed similar binding ability towards three targeted peptides, while no obvious fluorescence intensity was observed from TfR aptamer, indicating the conserved specificity of Tau aptamer in the construct. Results thus showed that Tau aptamer (IT2a) could selectively recognize two phosphorylatable peptide epitopes of Tau protein (T231, S202) and one phosphorylated peptide epitope (T231P).

The targeting capability of Tau aptamer in the circular construct towards full-length Tau protein was also confirmed using electrophoretic mobility shift assay (EMSA).

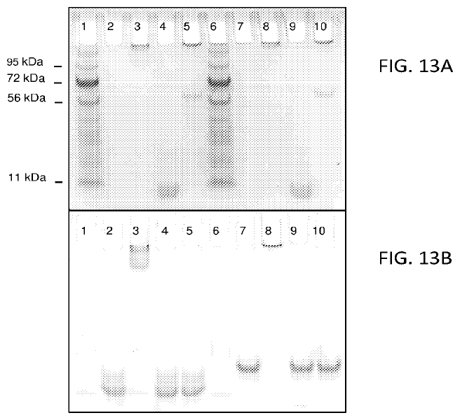

FITC-labeled IT2a or circular Tau-TfR aptamer was first incubated with targeted Tau protein (Tau441) or nontarget control proteins (bovine serum albumin (BSA) and astroglial calcium-binding protein (S100B)), respectively. After interaction, each sample was loaded onto the nondenaturing gel for protein detection with Coomassie Brilliant Blue staining (FIG. 13A) and aptamer fluorescence (FIG. 13B) detection. These figures show the specificity of circular aptamer against Tau441 protein. Five microliters of 400 nM Tau (IT2a) aptamer or circular aptamer was first incubated with 0.2 mg/mL target Tau441 protein or control proteins at 4° C. for 30 minutes. Without protein interference, the IT2a aptamer or circular aptamer alone showed a major band on the gel bottom (lane 2 and 7), while some minor upper bands were captured for IT2a, most likely from its multimeric transformations. For FIG. 13A and FIG. 13 B, Lanes 1 and 6: Ladder; Lane 2: IT2a aptamer; Lane 3: IT2a aptamer+Tau441 (67 kDa); Lane 4: IT2a aptamer+S100B (9 kDa); Lane 5: IT2a aptamer+BSA (66 kDa); Lane 7: Circular aptamer; Lane 8: Circular aptamer+Tau411 (67 kDa); Lane 9: Circular aptamer+S100B (9 kDa); Lane 10: Circular aptamer+BSA (66 kDa).

The control protein S100B has been reported as an important brain damage-associated biomarker as well, but no retardation of migration occurred on gel for the circular Tau-TfR aptamer after its interaction with the control proteins BSA or S100B (lane 9 and 10). However, a significant retardation was observed after reacting with Tau441, indicating that the specific complex only formed between circular Tau-TfR aptamer and Tau441 protein (lane 8). The binding profile of circular Tau-TfR aptamer was exactly the same as that of IT2a aptamer.

Therefore, the circular Tau-TfR aptamer maintained the selectivity to the targeted full-length Tau441 protein, laying the foundation for the following tauopathy treatment study. Moreover, the two FITC-labeled circular aptamers exhibited stronger binding shift due to the multivalency, suggesting the possibility of modifying circular Tau-TfR aptamer with more effective fluorescence tags for promising Tau protein detection in biological samples.

To deliver Tau aptamer across the BBB via aptamer-TfR complex-mediated transcytosis, it was imperative to ensure that the TfR aptamer was still functioning well in the circular construct. The immortalized mouse cerebral endothelial cells (bEnd.3) have been used to establish the in vitro BBB model as a surrogate to study the BBB permeability in different conditions. The targeting ability of circular Tau-TfR aptamer against the TfR-expressed bEnd.3 cells and the control HEK293T cells was investigated.

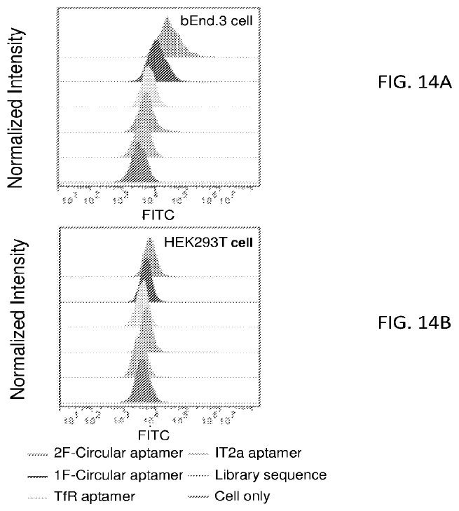

Flow cytometry and confocal microscopy analysis demonstrated specific binding and internalization of circular aptamer in the in vitro BBB model based on bEnd.3 cells. Different FITC-labelled_aptamer samples in 200 μL 400 nM were incubated with murine endothelial bEnd.3 cell (an in vitro model of BBB-linked epithelial cells) or HEK293T cell at 4° C. for 30 minutes. Circular aptamer with 1- or 2-FITC labels selectively bound to TfR-positive bEnd.3 cells and created greater shifts, but not TfR-negative HEK293T cells. See FIG. 14A and FIG. 14B. In comparison with TfR aptamer or library DNA sequence, the circular Tau-TfR aptamer showed better binding shift towards the target bEnd.3 cells, especially the two FITC-labeled circular Tau-TfR aptamer (FIG. 14A and FIG. 14B).

The improved binding ability most probably resulted from the strengthened closed-aptamer structure, as well as the rigid double-stranded complementary sequence domain that facilitated the formation of aptamer binding structure. Moreover, as there was no significant fluorescence enhancement in the flow analysis with HEK293T cells, the binding ability of circular Tau-TfR aptamer proved to be specific.

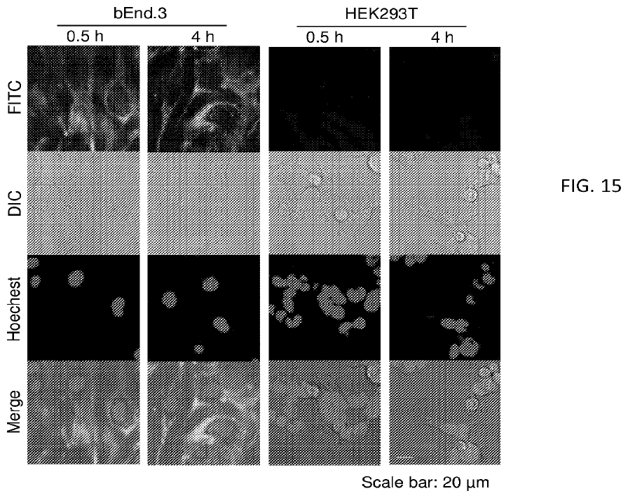

Transcytosis of cyclized Tau and TfR aptamers in the endothelial cells of BBB depends on internalization efficiency of the cargo. Confocal microscopy imaging was performed to analyze the cellular uptake ability of circular Tau-TfR aptamer in the TfR-positive and negative cells. The circular aptamer was incubated with bEnd.3 cells or HEK293T cells for 0.5 hours or 4 hours separately. As shown in FIG. 15, strong FITC fluorescence was localized in the cytoplasm of bEnd.3 cells after both 0.5 hour and 4 hour incubations, while almost no signal was observed from HEK293T cells, suggesting that circular Tau-TfR aptamer could rapidly internalize into the target cells and maintained the internalization ability for a long time.

### Example 8. Transport Efficiency of Circular Tau-TfR Aptamer Across the Blood-Brain Barrier

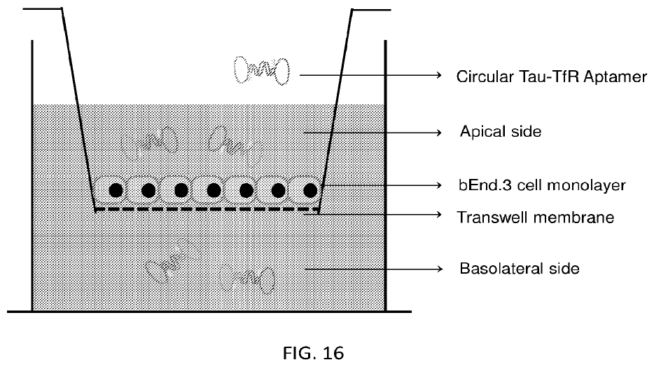

As previously discussed, in order to deliver the Tau aptamer into brain for tauopathy treatment, any aptamer complex would have to first penetrate the BBB. The data shown here, that the circular Tau-TfR aptamer could specifically internalize into the TfR-positive bEnd.3 cells. The next step was to investigate the penetration efficiency of the circular aptamer across the bEnd.3 cells-based BBB model before in vivo studies. See FIG. 16. Basically, a restrictive and compact bEnd.3 cell monolayer was formed on the transwell membrane after 9 days of culture, according to the literature. To assess the integrity of monolayer barrier, different-sized dextrans have been commonly used as the negative tracer molecules. Since the molecular weight of our circular Tau-TfR aptamer was around 30 k Da, we chose 10 kDa and 70 kDa TAMRA-tagged dextrans as the negative controls.

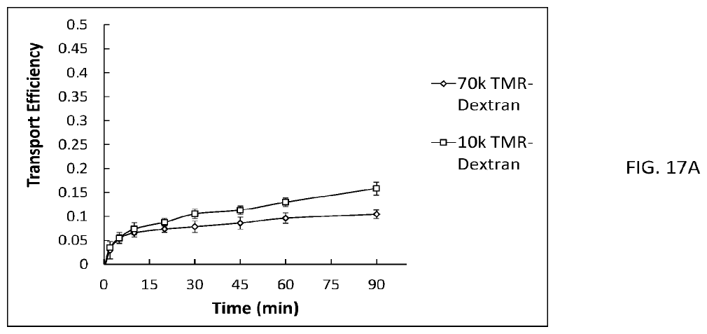

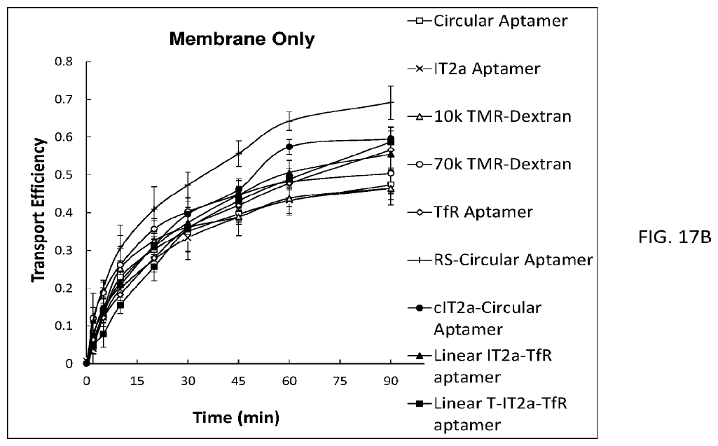

Fluorescence signal from the basolateral channel at each time point was detected for the calculation of real-time transport efficiency since the concentration of transferred cargo from upper to lower side was in direct proportion to the fluorescence intensity. After a 90-minute incubation with cell monolayer, transport efficiencies of 70 kDa and 10 kDa TAMRA-dextran were only 10%±1% and 15%±1%, respectively (see FIG. 17A). However, when incubated with transwell membrane alone for 90 minutes, the transport efficiency for either dextran was up to around 50%±5% (FIG. 17B). Data are means±SD (n=3). Therefore, the ratios between the transport efficiency in cells and membranes were around 20%±4% for 70 k dextran and 30%±3% for 10 k dextran. Consequently, these low transport ratios from controlled TAMRA-dextrans indicated the formation of a tight cell monolayer for BBB in vitro.

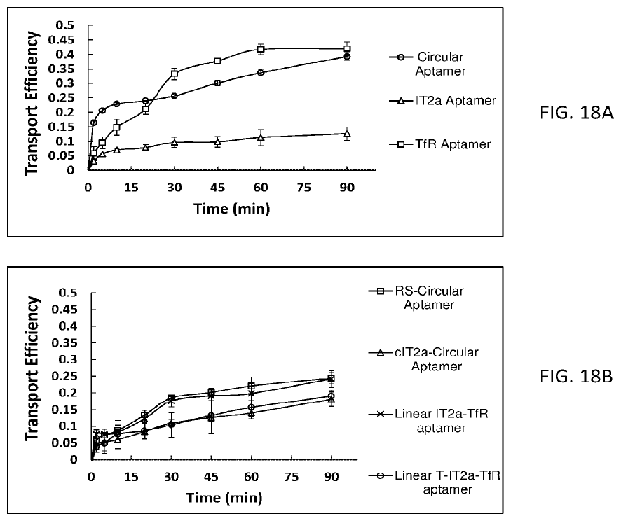

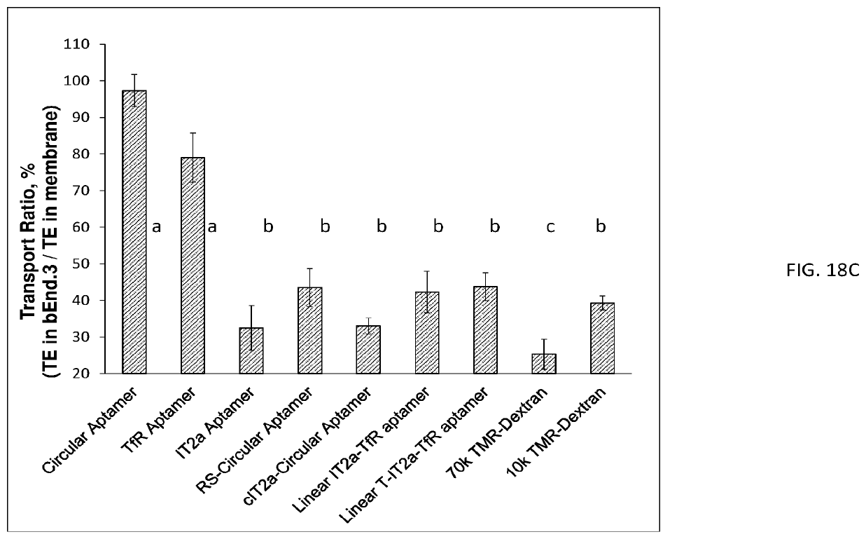

The next step was to examine the permeability of circular Tau-TfR aptamer in the tightly developed BBB cell model. 10 k Da and 70 k Da TAMRA-tagged dextrans were used as the negative controls. Fluorescent signal from the basolateral channel at each time point was detected for the calculation of real time transport efficiency. The transport efficiency of 10 μg/mL circular Tau-TfR aptamer quickly rushed to 20%±1% within only 5 min, 2-fold higher than that of single TfR aptamer, while both efficiencies finally increased to a plateau of around 40%±2% after 90 min incubation. Results are shown in FIG. 18A and FIG. 18B. Transport Efficiency=(Fₜᵢₘₑ−F_{medium})/(Fₜₒₜₐₗ−F_{medium}). Data are means±SD (n=3). **P<0.01, ***P<0.005.

Interestingly, the transport efficiency of 10 μg/mL circular aptamer quickly rose to 20%±1% within only 5 minutes, 2-fold higher than that of single TfR aptamer, while both finally increased to a plateau of around 40%±2% after a 90 minute incubation (see FIG. 18A and FIG. 18B). The burst for transcytosis of circular aptamer at the beginning was a reflection of the strengthened structure that facilitated enhanced targeted cellular uptake, but as time passed by, more and more TfR aptamers were also captured by TfR on the cell membrane, gradually transcytosing to the basolateral side. In contrast, lower transport efficiency was obtained after a 90 minute incubation with other controlled aptamers (compare FIG. 18A and FIG. 18B), including Tau aptamer alone (12%±2%), circular aptamer constructed by random sequences (24%±2%), circular aptamer constructed by two Tau aptamers (18%±2%), linear Tau-TfR aptamer containing complementary sequences (24%±2%), and linear Tau-TfR aptamer containing poly-T instead of complementary sequences (19%±2%).

These results ruled out the possibility that Tau aptamer, or the size of a DNA construct, played a role in facilitating transcytosis. However, linearly synthesized bifunctional Tau-TfR aptamers showed only modest transport efficiency. One possible reason might be that the longer linear bispecific aptamers were less stable and tended to form unexpected self-dimers, which would affect desirable targeting conformation of TfR aptamer.

Moreover, after an 8 hour incubation, final transport ratios were calculated for all aptamer groups. See results in FIG. 18C, which shows the transport ratio (%) of each sample, as normalized by each TE in transwell membrane after 8 hours of incubation. Data are means±SD (n=3). Different lowercase letters within rows indicate the significant difference at the 5% level (P<0.05).

Circular Tau-TfR aptamer had a final fluorescence intensity similar to that of TfR aptamer in the bottom channel of the BBB cell model, but lower signal after passing membrane alone compared to TfR aptamer (data not shown), probably owing to the higher permeability of membrane towards smaller and linear TfR aptamer. Accordingly, calculated by transport efficiencies from cell monolayer and transwell membrane alone, circular Tau-TfR aptamer showed 97%±4% of transport ratio, while TfR aptamer showed 79%±7%. Both were significantly higher than the transport ratios of controls, indicating that effective penetration across BBB requires a stabilized TfR aptamer conformation. Transport ratio calculation is just one method of demonstrating the permeability of circular aptamer, but based on the present results, it is possible to conclude that the circular Tau-TfR aptamer could pass through the in vitro BBB model efficiently and that circularized and stabilized TfR aptamer played a predominant role.

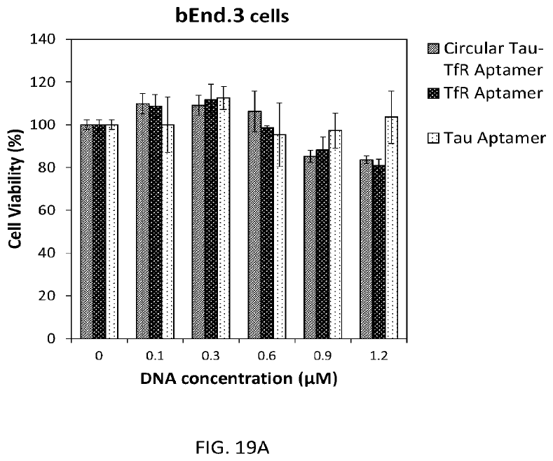

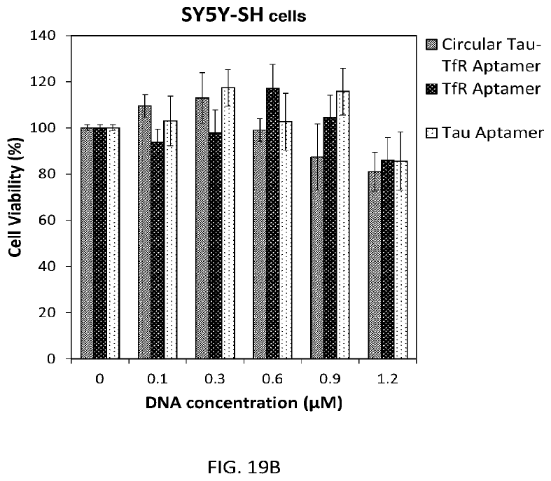

Simultaneously, consistent internalization results were obtained from flow cytometry, as only intracellular aptamers could give fluorescence signals after removing ligands from the cell surface using trypsinization (FIG. 11). The stabilized circular aptamer displayed slightly enhanced cellular uptake in bEnd.3 cells compared to TfR aptamer alone and kept similar uptake capability with prolonged incubation time. Importantly, neither the circular aptamer nor the single aptamer showed obvious cytotoxicity towards brain endothelial cells (bEnd.3) or neuroblastoma cells (SY5Y-SH), suggesting good biocompatibility. See FIG. 19 and discussion below. Consequently, the noninvasive circular Tau-TfR aptamer exhibited efficient targeted cellular delivery, which would be beneficial for its transport across BBB.

### Example 9. Transport Study in a Constructed In Vitro BBB Cell Model

To study the transport efficiency of circular Tau-TfR aptamer across in vitro BBB cell model, a monoculture system was established according to methods known in previous literature with modifications. Briefly, transwell membrane inserts were first placed in the wells of 12-well culture plates and moistened with 1.2 mL bEnd.3 cell culture medium in the basolateral side of each well. Then the bEnd.3 cells (2.5×10⁵) were seeded in the apical side of transwell inserts and cultured at 37° C. in 5% CO₂. The cell culture medium (0.5 mL) was changed every other day and the cells were grown for 9 days to make a very compact cell monolayer.

After removing the entire medium from two sides, 1 mL fresh full cell culture medium was replaced in the basolateral side of the BBB cell model, while 10 μg/mL of each DNA sample in 0.5 mL fresh full cell culture medium was added to the apical chamber and was incubated with cell monolayer for 0, 2, 5, 10, 20, 30, 45, 60, and 90 minutes, and 8 hours. At each time point, the fluorescence signal from basolateral side medium containing different samples was detected by a CLARIOstar™ plate reader (BMG LABTECH™). Full culture medium was used as the blank control, and the 70 k and 10 k (MWCO) TAMRA™-labeled dextrans were used as negative controls. See results in FIG. 19, which presents data for a cell viability test of bEnd.3 cells and SY5Y-SH cells incubated with TfR aptamer, Tau aptamer and circular aptamer at different concentrations. Data are means±SD (n=3).

Since the fluorescence intensity was in direct proportion to the amount of each DNA sample, the transport efficiency (TE) of each sample across the BBB cell model was calculated as TE=(Fₜᵢₘₑ−F_{medium})/(Fₜₒₜₐₗ−F_{medium}), where Fₜᵢₘₑ, Fₜₒₜₐₗ and F_{medium} represent the fluorescence signals from the basolateral side medium containing different samples at each time point, the basolateral side medium containing the entire 10 μg/mL of each DNA sample, and the blank control medium, respectively. Furthermore, the transport ratio (%) of each sample, as normalized by each TE in transwell membrane only, was calculated after 8 hours of incubation. All experiments were performed at least three times. The results showed that circular Tau-TfR bispecific aptamers had the ability to transport across bEnd.3 cell monolayer in the in vitro BBB cell model. See FIG. 19.

### Example 10. Biodistribution and Ex Vivo Imaging of Mice

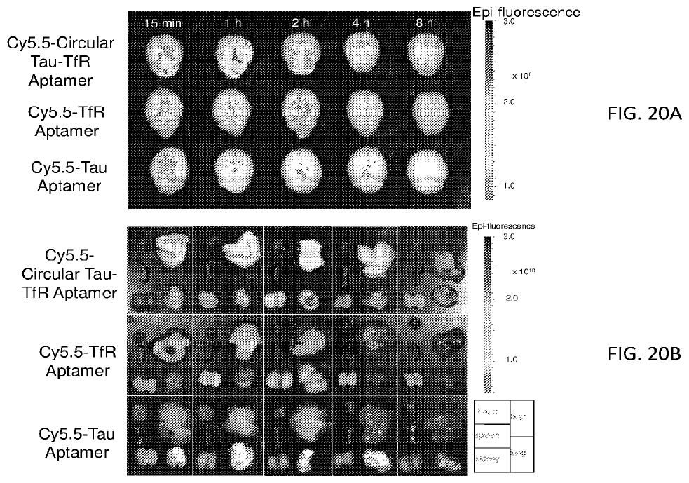

Having demonstrated the improved penetration ability of circular Tau-TfR aptamer across the cell-based BBB model, we then compared the brain targeting performance of Cy5.5-labeled circular Tau-TfR aptamer, Tau aptamer and TfR aptamer in an animal model. To study the biodistribution of circular Tau-TfR aptamer, healthy C57/BL6 mice (average weight 25 g) were anaesthetized using an isoflurane vaporizer and perfused with 1×PBS after retro-orbital injection of 150 μL (120 nmol/kg) of Cy5.5-labeled circular Tau-TfR aptamer for varying time periods (15 minutes, and 1, 2, 4 and 8 hours) for comparison to TfR aptamer. The brain, heart, liver, spleen, lung, and kidney were carefully collected to detect fluorescent intensity (see FIG. 20A and FIG. 20B) using the PerkinElmer IVIS Spectrum Bioluminescent Imager (excitation: 640 nm, emission: 700 nm). The mean fluorescence signals from different organs were analyzed using PerkinElmer Living Image v4.3 software.

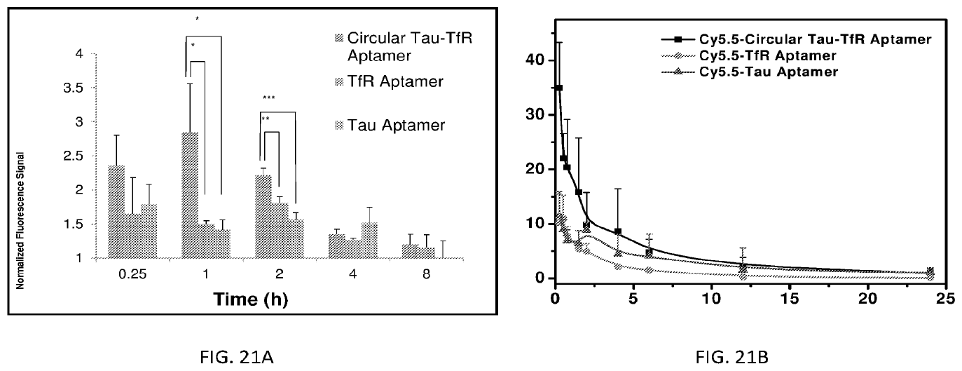

A rapid accumulation of fluorescent signal from circular Tau-TfR aptamer was observed in the brain within 15 minutes, and the fluorescence was distributed throughout the whole brain post 1 hour injection, and maintained a high level after 2 hours. A relatively low amount of TfR aptamer penetrated the brain after a 15-minute time period, but a slightly increased amount was distributed in brain after 2 hours. Thus, the difference between circular Tau-TfR aptamer and TfR aptamer indicated that circularization of the aptamer promoted TfR aptamer-mediated specific transport across the BBB in vivo. In the case of Tau aptamer, some uptake was detected after the 15-minute administration, whereas only low signal could be collected for the following time points, suggesting nonspecific transport of Tau aptamer into brain right after the injection. As shown in FIG. 21A (quantitative analysis of the normalized fluorescent signal of the brains), the average fluorescent signals in brains were normalized and quantified as well. *P<0.05, **P<0.01, ***P<0.005.

Consistently with this, circular Tau-TfR aptamer was quickly taken up by the brain and was located in brain with remarkably higher amount than TfR or Tau aptamer 1 hour and 2 hours after injection. The stronger signal and longer retention in brain of circular aptamer could be attributed to two major factors: the more robust and stable DNA structure and the accordingly enhanced selective targeting ability of TfR aptamer.

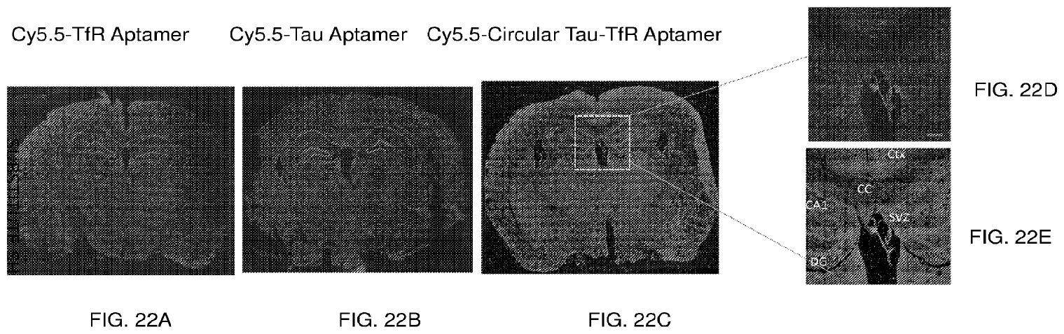

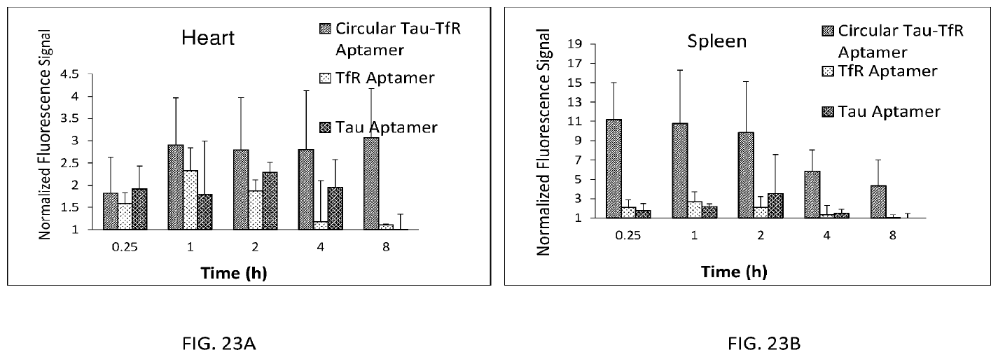

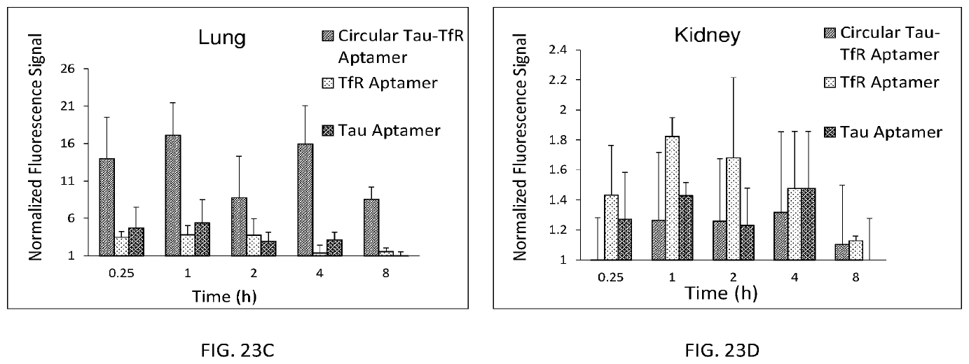

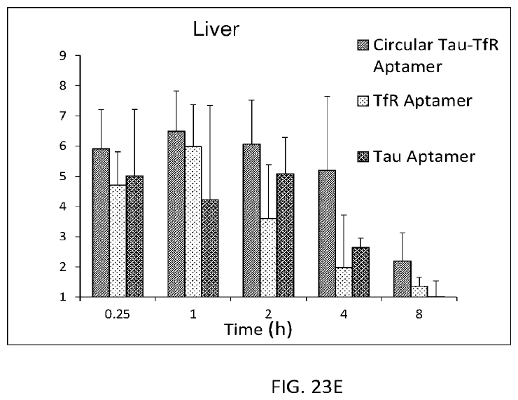

At the same time, biodistributions of different aptamer samples in other major organs were also investigated, and the aptamers were mainly located in the kidneys and lungs (see FIG. 20A and FIG. 23). After the 15-minute administration, the fluorescent intensity of circular Tau-TfR aptamer was much lower than that of the linear parent TfR or Tau aptamer in kidney, but gradually increased after injected for longer time, whereas stronger signal was observed in liver from circular Tau-TfR aptamer rather than TfR or Tau aptamer for all the time points. This result indicated that the circular Tau-TfR aptamer had a longer retention in the body, while the smaller and less stable single stranded TfR or Tau aptamer was more easily digested and cleared out more quickly. This was also indicated by the blood circulation profiles of the three aptamer samples in vivo (FIG. 21B). FIG. 22A through FIG. 22E are confocal images of entire dissected brain slices from mice treated with Cy5.5-labeled aptamers for 1 hour. Brain sections were stained with cell nucleus DAPI dye (blue), while aptamer was visualized by NIR Cy5.5 fluorescence (red). Cy5.5-labeled circular Tau-TfR aptamer showed superior penetration and residency in brain tissues, e.g., in subventricular zone (SVZ), hippocampus (CA1), dentate gyrus (DG), cortex (Ctx) and other areas. Scale bar: 200 μm. Data are means±SD (n=3). The results show that the circular Tau-TfR Aptamer-Cy5.5 shows superior penetration and residency in brain tissue, e.g., in the subventricular zone, hippocampus and other areas.

Mouse blood also was collected at a series of time points after aptamer injection, and the Cy5.5 fluorescence intensity was detected to determine the corresponding aptamer concentration in blood. In agreement with the ex vivo imaging results, the residual content of circular Tau-TfR aptamer in the body after the 15-minute injection was about 35% ID g-1,3.5-fold higher than that of TfR or Tau aptamer, and an obvious content difference existed continuously after blood circulation over time. Therefore, the circularized Tau-TfR aptamer possessed significantly improved biostability, thereby prolonging its circulation time in vivo.

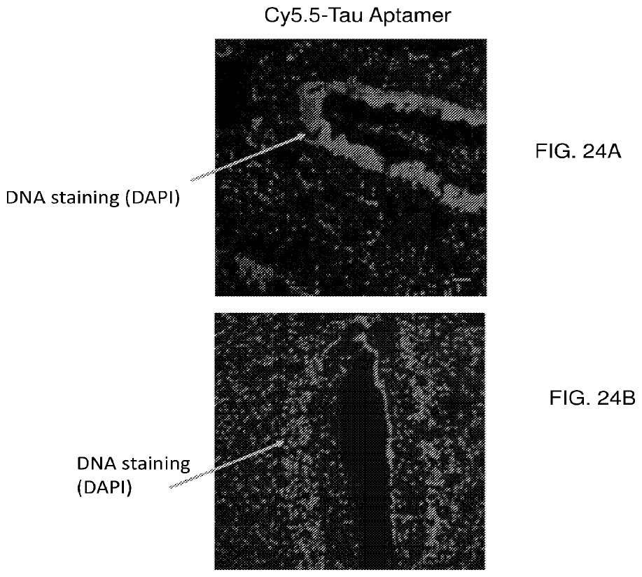

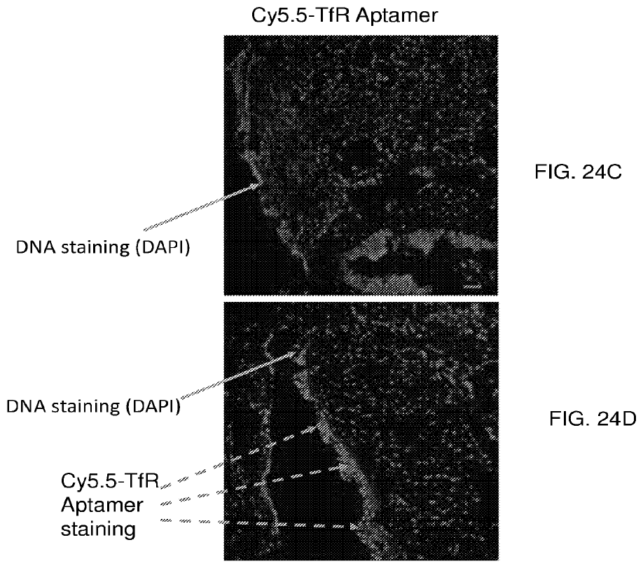

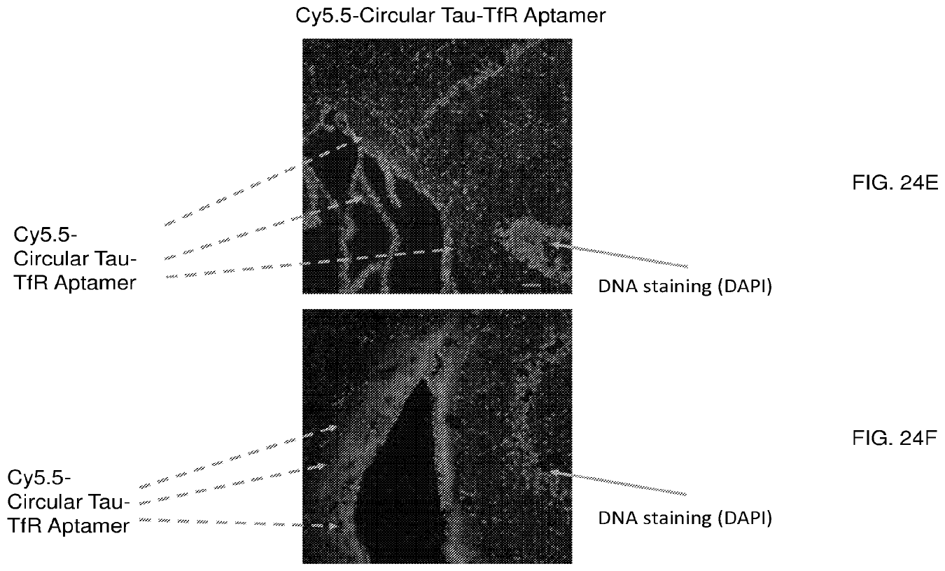

Compared to the in vitro BBB cell model, it is noteworthy that a greater differentiation of BBB penetration ability between circular aptamer and TfR aptamer was generated in the in vivo BBB model, which could likely be correlated with the more complex physiological environment. Furthermore, the fixed brains from mice injected with aptamers for 1 hour were dissected, and the slices were scanned by confocal microscopy to study the localization of aptamers in brain (see FIG. 21B and FIG. 24).

In FIG. 24, brain slices were counterstained with the DNA binding dye DAPI. Circular Tau-TfR aptamer was mainly located in the areas near brain ventricles, especially the cortex and hippocampus, which are thought to be highly involved in tauopathy formation. In contrast, the BBB was nearly impermeable to the Tau aptamer or TfR aptamer after 1-hour administration. Hence, together with all the imaging results, the results show that circular Tau-TfR aptamer could be actively transported across the BBB into important brain regions related to Tau pathology. This indicates that the aptamers can be used to reach the cells needing tauopathy treatment.

### Example 11. Neuronal Surface Adhesion Molecule L1CAM with Tau Targeting Bispecific Aptamer or with Tau and TfR Trispecific Aptamer Improves Neuronal Cell Uptake of Aptamer

To overcome the neuron cell barrier and suppress the intracellular Tau aggregates, aptamers targeting neuron cell membrane proteins can be included to make a bispecific or trifunctional aptamer nanostructure, such as anti-L1CAM aptamer. As shown in FIG. 5, anti-L1CAM aptamer

(5′-AGGATAGGGGGTAGCTCGGTCGTGTTTTTGG
GTTGTTTGGTGGGTCTTCTG-3′; SEQ ID NO: 19)

could be conjugated with Tau aptamer to form circular Tau-L1CAM bispecific aptamer in order to deliver the Tau aptamer into neuron cells for intracellular tauopathy treatment; or conjugated with both Tau and TfR aptamers to form Tau-TfR-L1CAM trispecific aptamer in order to fulfill the needs for BBB and neuron cell membrane penetrations.

We hypothesized that the circular Tau-L1CAM bispecific aptamer can enable the internalization of Tau aptamer into neuron cells through the L1CAM protein-mediated endocytosis. In this case, the Tau aptamer can inhibit the intracellular aggregated Tau deposits and further attenuate the tauopathy. At the same time, by adding TfR aptamer, the resulted trispecific aptamer can cross both BBB and neuron cell membrane barriers, which will be practically applied into the real in vivo models for tauopathy studies.

### Example 12. Methylene Blue and Other Protein Aggregation Inhibitor-Conjugated Tau Aptamers to Improve Tau Aggregate Disruption

Many Tau-directed therapeutic interventions have been proposed for treating tauopathies, such as stabilizing microtubule or preventing Tau aggregation using chemotherapy drugs. However, the Tau aggregation inhibitors (e.g. methylene blue, MB) have been reported to lack targeting specificity as they bind to hydrophobic domains of multiple proteins. Here, introducing Tau aggregation suppressor onto the circular Tau-TfR aptamer via chemical modification might improve the specificity and binding between drug and aggregate, representing better synergistic effects on tauopathy treatment.

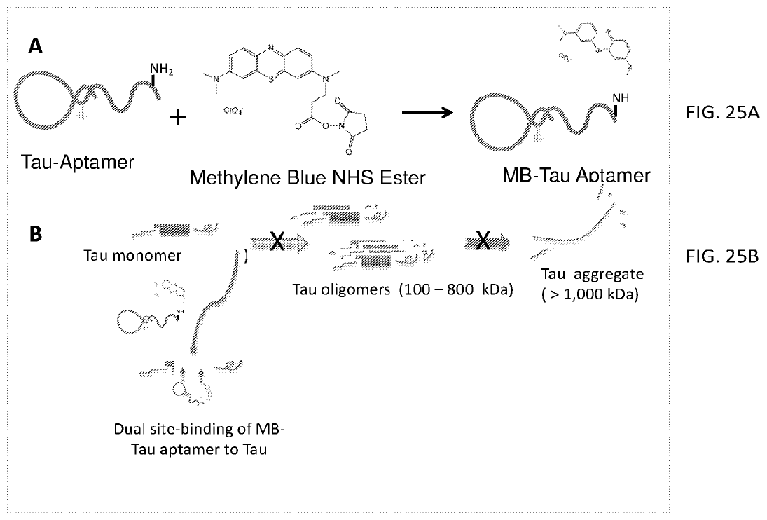

The amine-modified circular Tau-TfR aptamer is constructed first, following with the conjugation of NHS ester-modified methylene blue (MB). See FIG. 25A. Once the Tau aptamer binds to the Tau monomer, the attached MB can bind to the hydrophobic domain on Tau due to the close proximity and result in the suppression of Tau aggregation. See FIG. 25B. This effect can be synergistic, since both Tau aptamer and MB showed inhibitory effects on Tau aggregation. More designs could be studied on the aptamer-MB linkages. The length (e.g. PEG, poly-T) and fragility (e.g. disulfide bond) of the linkers would be considered to allow better performance of the MB.

### Example 13. Conjugation to Gd3+Chelator (e.g. DOTA-Gd3+) for MRI-Imaging of Tau Aggregate In Vivo

Many imaging techniques, such as X-ray computed tomography (CT), ultrasonography (US), positron emission tomography (PET), and magnetic resonance imaging (MRI), are currently available for the diagnosis of diseases. Gadolinium ion (Gd3+) complexes are commonly used as MRI contrast agents. MR signals of tissues in which Gd3+ ions have accumulated show high intensities in T1-weighted MR images. However, many MRI compounds (e.g., DOTA-Gd) do not penetrate in the brain spontaneously and require the use of invasive techniques (e.g., BBB transient opening using hyperosmotic agents or ultrasound-associated microbubble injections) to reach their target. Moreover, gadolinium (Gd3+) contrast MRI has no selectivity to Tau. Therefore, coupling Tau/TfR1 circular aptamers to (Gd3+) chelator “cage” DOTA-moiety can help facilitate the penetration of DOTA-Gd complex into brain and its target imaging of Tau.

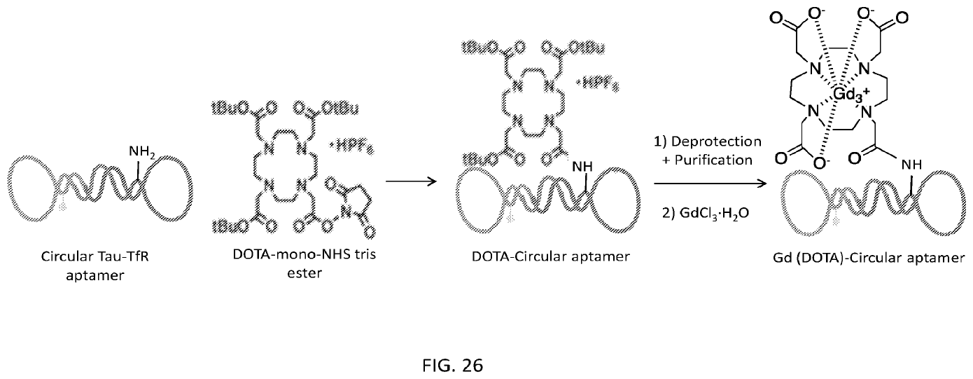

Similar to the MB-circular aptamer conjugation, here, the amine-modified circular Tau-TfR aptamer is conjugated with NHS ester-modified DOTA (see FIG. 26). After HPLC purification, the tert-butyl protective groups on the DOTA-circular aptamer complex can be removed using 20% TFA in DCM at room temperature for 12 hours. Then, the sample is tested by HPLC for another purification. Finally, the GdCl₃.H₂O is loaded into the complex to form the DOTA-Gd-circular aptamer imaging probe. The pure product then can transport across BBB in vivo and specifically recognize Tau, to help the selective MRI imaging on Tau protein or aggregations.

### Example 14. Protective Effects of Circular Tau-TfR Aptamer on Reducing TBI-Related Pathological Biomarkers Levels

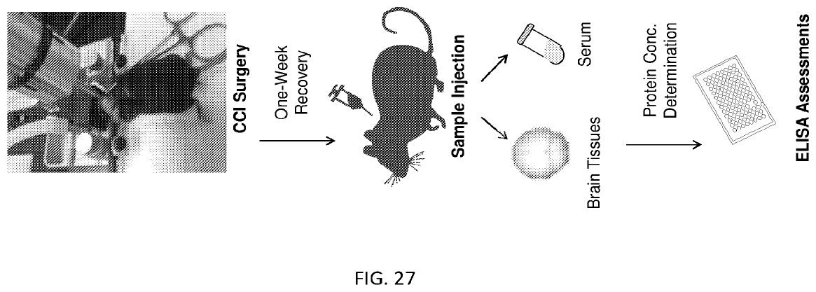

Traumatic brain injury (TBI) has been a major cause of disability and death, and it has therefore raised public awareness worldwide. Repetitive TBI can result in increased accumulation of total Tau (T-Tau) and hyperphosphorylated Tau (P-Tau), as well as their pathological aggregations in brain neurons, leading to tauopathy diseases. Since the Tau aptamer described has shown the ability to inhibit Tau phosphorylation and oligomerization in vitro, whether the circular Tau-TfR aptamer could be useful as a therapeutic candidate for alleviating tauopathy-associated disorders in animal models (given its sustained binding ability to phosphorylated or non-phosphorylated regions on Tau protein) was investigated. To evaluate the possible therapeutic effect of circular aptamer on tauopathy treatment, three sets of transgenic human Tau-overexpressing (hTau) mice were used in this study (see FIG. 27).

Controlled cortical impact (CCI) surgery was performed on all mice to induce TBI and to increase neuropathological biomarkers for comparison before and after aptamer treatment. After one week of recovery, the three sets of mice (randomly assigned) were treated with nothing (negative control), Tau aptamer, or circular bispecific aptamer, in three experimental groups. The aptamers (200 nmol/kg) were administered once a day for five days with the same dosage to ensure a continuous level. Finally, brain tissue lysates and serum samples from CCI mice with or without aptamer treatments were collected to analyze the protein levels of TBI-related biomarkers using enzyme-linked immunosorbent assay (ELISA).

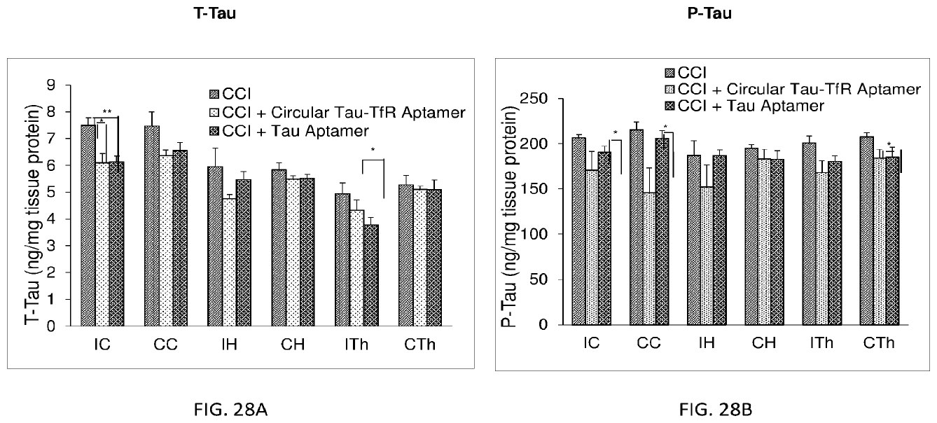

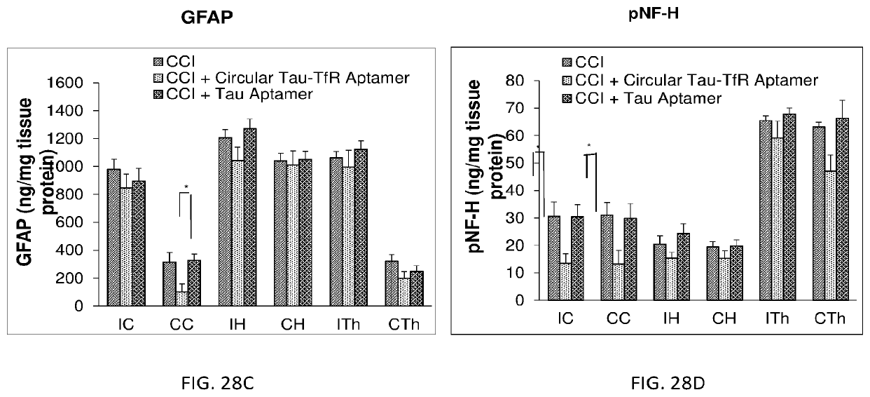

Because of augmented Tau aggregation in tauopathy brains, T-Tau and P-Tau isolated from both ipsilateral (injured) and contralateral (uninjured/controlled) brain tissues were evaluated. MSD homebrew ELISA assays were developed to determine the concentrations of T-Tau and P-Tau proteins. Both circular aptamer and linear Tau aptamer showed significant reduction on T-Tau levels in the ipsilateral cortex post-injury, while only the circular aptamer decreased P-Tau levels significantly in both injured and control cortex. See FIG. 28A and FIG. 28B. Mild changes were also observed for either T-Tau or P-Tau levels from hippocampus and thalamus after aptamer treatments. Tau proteins and major Tau kinases have brain region-specific expression with highest levels in cortex and hippocampus, where tauopathy lesions start and spread. Because the cortex and hippocampus are known to be critical brain regions for the formation of memory, but are more vulnerable to Tau pathology, the alleviation of T-Tau and P-Tau elevations in these brain areas by circular aptamer may provide a proof-of-principle foundation for the potential use of Tau-targeting aptamers in the clinical treatment for TBI-associated tauopathies.

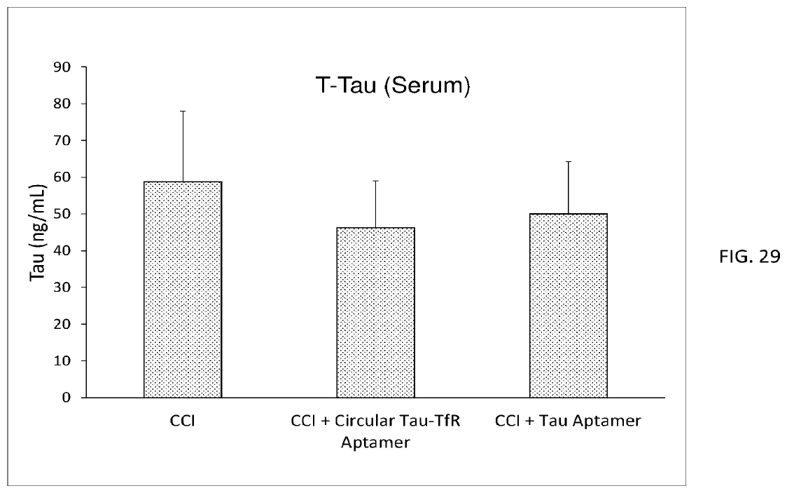

A slight decrease in serum T-Tau level was noted after circular aptamer administration at the end of the five-day aptamer treatment (12 days post-injury, see FIG. 29). However, more robust detection tools are required in the future for serum T-Tau and P-Tau detection at such delayed time point to non-invasively assess the beneficial effects of circular aptamer. The protein levels of additional neuropathological biomarkers, including astrogliosis glial fibrillary acidic protein (GFAP) and phosphorylated axonal form of the heavy neurofilament (pNF-H), in brain tissues were also measured after different aptamer treatments. Results are shown in FIG. 28C and FIG. 28D. For FIG. 28, the data are means±SEM (n=5-6). *P<0.05, **P<0.01. IC/CC, injured/control cortex; IH/CH, injured/control hippocampus; ITh/CTh, injured/control thalamus.

GFAP and pNF-H are important glial cell injury and axonal injury markers, respectively, and were found to be elevated in brain tissues in TBI animal models. Here, suppression on both biomarker levels was observed in cortex after brain injury with circular aptamer injection, indicating that circular aptamer not only could reduce its targeted T-Tau and P-Tau proteins, but also could attenuate other TBI-related neuropathological protein markers. Overall, circular Tau-TfR aptamer has behaved as a potential drug candidate with protective effects on decreasing elevated TBI-related neuropathological protein levels.

### Example 15. Effects of Circular Tau-TfR Aptamer on Memory in Mouse Fluid Percussion Injury Model

The suppression of Tau has been found to improve memory function in a mouse model of neurodegeneration. To further examine whether circular Tau-TfR aptamer has therapeutic effects on improving cognitive deficits and memory impairment, a mouse fluid percussion injury (FPI) model that produced impaired memory functions was used, followed by measurement using a Y-maze test.

The fluid percussion injury (FPI) model was used based on previous literature descriptions. Tg(MAPT)8cPdav/J (hTau) mice (4-6 months old, 25-30 g) were anaesthetized using an isoflurane vaporizer. A midline cranial incision then was made on the head (1-2 cm), and a unilateral craniotomy (˜3 mm diameter) was performed adjacent to the central suture, midway between Bregma and lambda. A plastic tube was placed on the exposed dura and glued onto the skull using dental acrylic. Afterwards, FPI was induced by a pressure pulse of 1.0-1.5 atm intensity through the connection with the plastic tube on the mouse head/brain filled with saline.

After surgery, mice were placed back in their home cages and monitored closely. The next day, mice were randomly assigned to three treatment groups: naïve control, FPI only control, and FPI+circular Tau-TfR aptamer, each group containing 8-12 mice. Mice in the aptamer treatment group were then anesthetized and injected or infused with 150 μL 200 nmol/kg of circular aptamer (as retro-orbital injection) once a week for five continuous weeks. During the treatments, mice were monitored consistently.

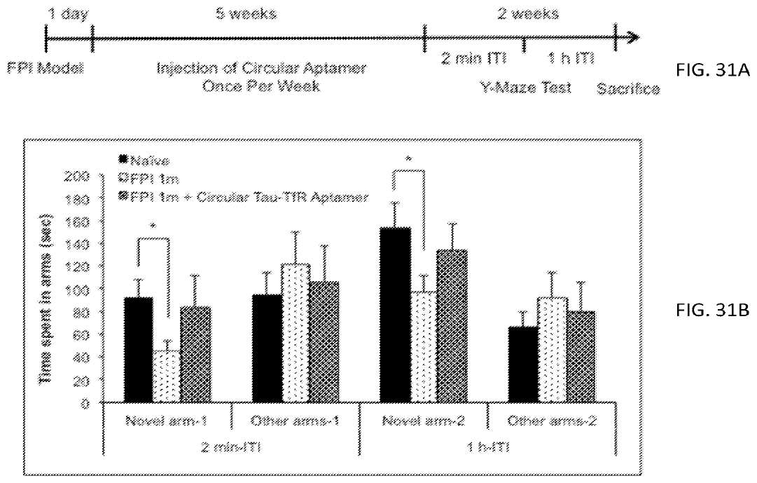

After finishing all the treatments, a two-trial Y-maze was used to test mouse response to novelty (a test of spatial memory). In brief, the study was performed in a room with dim light and low noise. Many cues were established around the maze, and they were maintained constant throughout the whole testing period, including computers, tables, chairs, curtains, buckets and some other small items. Mice were placed in a quiet room next to the experimental room with the same illumination and noise conditions to let them calm down before the experiments for at least one hour. Then, two experiments were used to test mouse response to novelty and spatial memory with 2-minute and 1-hour inter-trial intervals (ITI), respectively, within two continuous weeks. See FIG. 31A.

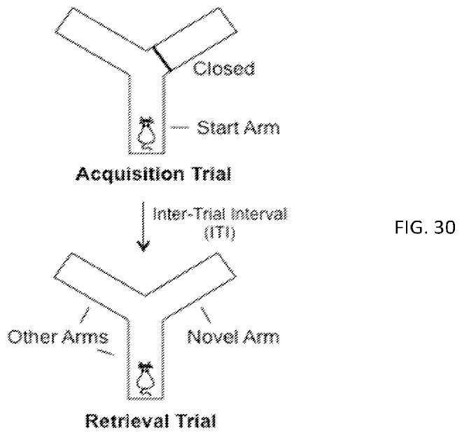

In experiment I (2-minute inter-trial interval, ITI), two trials were conducted with a 2-minute ITI. In the first acquisition trial, one arm of the Y-maze was selected randomly and blocked by a guillotine door (novel arm). Then the mouse was individually placed in one of the other two arms (start arm) with the head pointing away from the maze center. Each mouse was allowed to explore the two open arms for a 5-minute acquisition period. After 5 minutes, the mouse was put back in the home cage for 2 minutes. At the same time, the entire maze was wiped using H₂O₂ to remove odors from the previous mice. During the second retrieval trial, the guillotine door in the novel arm was taken away, and the mouse was placed in the start arm and was allowed to visit all three arms for 5 minutes. For each mouse in each trial, the durations of visits to all three arms were recorded. In experiment II (1-hour ITI), a longer ITI was used to analyze the spatial memory of each mouse based on the same procedures shown above. All behavior tests were performed in the same maze and same room with the same cues. The maze experiments are shown in a schematic in FIG. 30.

The cognitive behavior performance of three groups (naïve, FPI 1 month, FPI 1 month+circular aptamer) was evaluated based on arm discrimination using a Y-maze. The timeline for these tests is given in FIG. 31A. The 2-minute and 1-hour inter-trial intervals represented short and long retention times. The overall time each mouse spent in the novel arms versus the other two arms in the retrieval trial was noted.

The results of the tests are shown in FIG. 31B, including the overall time spent in the novel arm of the maze and the other arms after 2-minute or 1-hour inter-trial intervals. The data are means±SEM (n=8-12). *P<0.05. As shown in FIG. 31B, compared to naïve mice, FPI mice spent significantly less time in the novel arm, both after 2 minutes ITI and after 1 hour ITI, indicating the impairment in spatial memory after FPI surgery. This suggests that the untreated FPI mice regarded all arms as novel. In contrast, the circular Tau-TfR aptamer treated FPI mice showed an elevated amount of time spent in exploring the novel arm, rather than the other arms, in both the short and long retention time test. This suggests that circular aptamer treatment improves the cognitive ability of injured mice and enables them to differentiate the novel arm from the remembered arms. Taken together, these results demonstrated that the circular Tau-TfR aptamer has the effect of restoring memory deficits in the TBI mice model, and that continuous treatment may further ameliorate other cognitive and neurobehavioral dysfunction symptoms.

### Example 16. Identification of Two Series of Tau Aptamers with Tau Functional SELEX

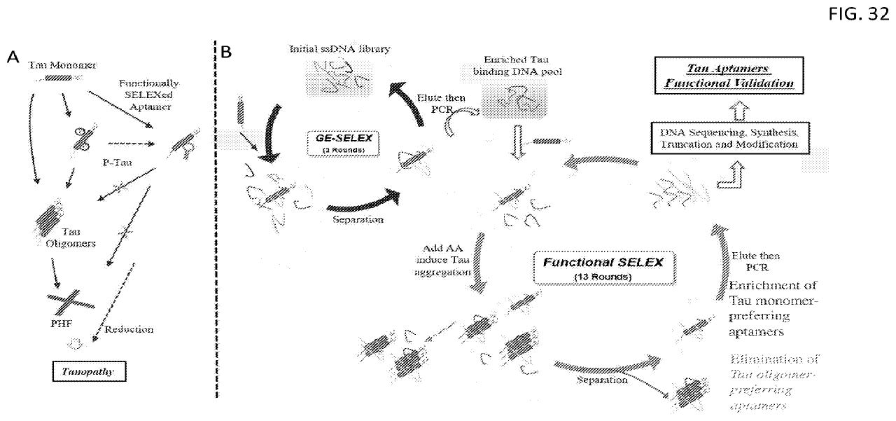

In this example, we designed and performed a novel “Functional SELEX” where the aptamers bound with the survived monomer Tau protein were separated in electrophoretic running system after being treated with the aggregation inducers. The function-guided molecular evolution demonstrated here efficiently generate the functional binders which specifically inhibit tau aggregation to a great extent in cell-free and cellular milieu. These results suggested that the identified aptamers with strong inhibitory effects could be further implemented as molecular probes and therapeutic agents simultaneously for a variety of bioapplications in the future. Moreover, this novel Functional SELEX system shows a straightforward avenue to researchers, in principle, to develop and expand the specifically demanding function and properties of aptamers. Overall, our approach was carefully designed to maximize selection of tau-binding aptamers that robustly prevents tau oligomer and the corresponding aggregate formation (FIG. 32).

Enriched Tau Binding DNA Pool Discovery.

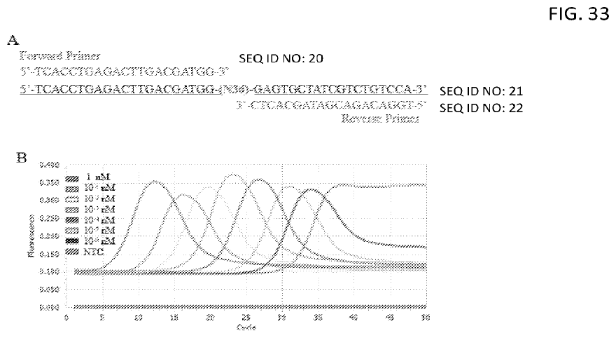

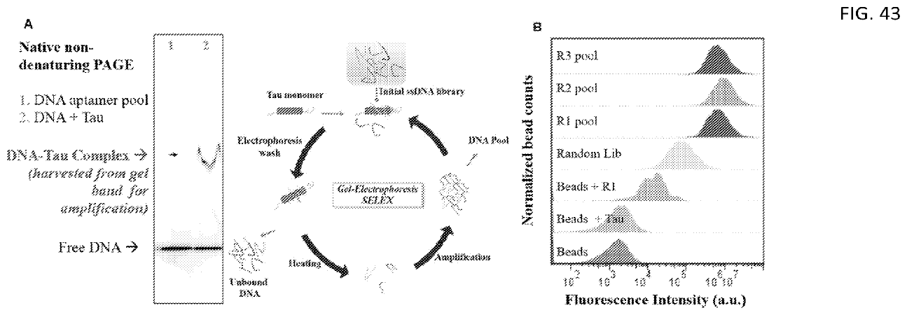

The GE selection employed a DNA library pool containing 76-nucleotide, single-stranded DNA with r36 nucleotide random sequences (FIG. 33). Monomer Tau 441 (441 amino acids long) was used as the target (FIG. 1A). The DNA library and primers were validated by qPCR. No template control (NTC) showed a positive signal indicating the nonspecific amplification of primer dimer only after 30 cycles. Compared to the cycle number used in amplification step in SELEX, high Ct value of primer pairs validated the low chance of heteroduplexes formation between the primer pairs. Therefore, these primer sequences were well suited for SELEX process. In the process of GE-SELEX, native non-denaturing PAGE gel electrophoresis was initially implemented to analyze FAM-labeled DNA library pool after incubation with Tau protein. DNA-Tau complexes exhibited much slower migration rate compared with free DNA due to the higher molecular weight, fewer negative surface charges, and larger physical size after binding. The bound complexes were easily identified in PAGE gel by FAM signal from DNA and efficiently harvested from gel band for PCR amplification. Unbound DNA with nonspecific or weak binding properties were washed away during the electrophoretic running (FIG. 43A). With this approach, GE strategy effectively separated the bound DNA candidates from unbound ones. After the enrichment of aptamer candidates from GE-SELEX, flow cytometry was implemented to further validate the progress of GE-SELEX. Here, the beads incubated with Round 1 DNA pool and beads incubated with either tau or random library were set as the control group. The flowcytometry shift compared with the control group indicated that the R1 (Round 1), R2 and R3 DNA pools had strong binding ability with the target Tau protein. (FIG. 43B).

Functional SELEX.

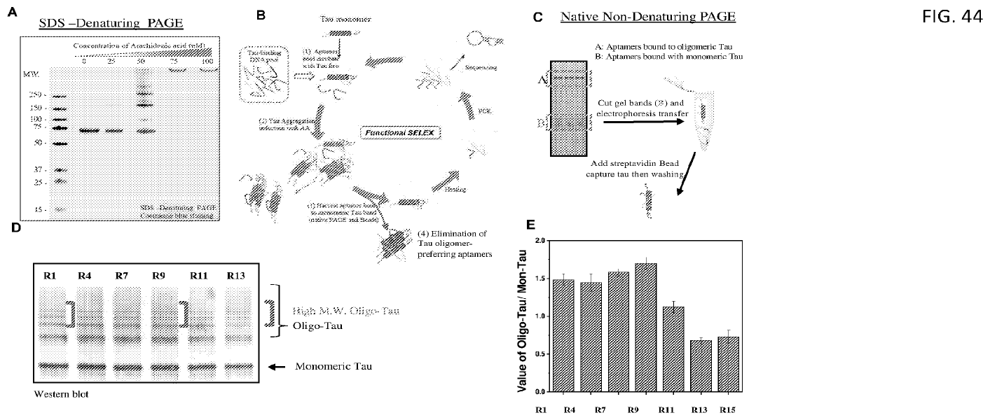

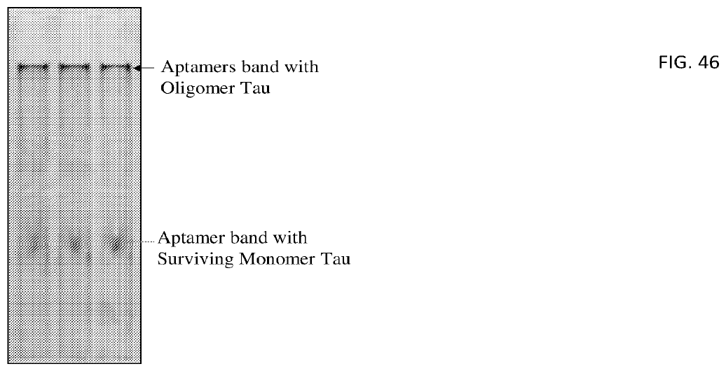

Function guided SELEX was then performed with the preselected and enriched Tau binding DNA pool after the validation of the overall binding ability of DNA library pool. In order to generate Tau aggregates in cell-free condition, we firstly attempted to utilize arachidonic acid and heparin. Arachidonic acid has demonstrated to be a great aggregate inducer promoting tau protein aggregation as indicated in protein denaturing SDS-PAGE gel clearly. Meanwhile, we investigated the effect of arachidonic acid's concentration on Tau protein aggregation with SDS-PAGE gel demonstrating that quantity of oligomerized tau proteins significantly increased in a dose-dependent manner of arachidonic acid inducer (FIG. 44A). After the assessment of aggregation inducers, function-guided SELEX was performed by incubating biotin labeled Tau441 monomer and the third round GE SELEX enriched Tau binding DNA pool for 30 mins at 37° C., followed by the additional incubation with arachidonic acid (20-100 uM) for 12 h at 37° C. (FIG. 44B). The Tau441 monomers were then separated from Oligo-Tau by native non-denaturing PAGE gel electrophoresis (FIG. 44C & FIG. 46). The band of Tau441 monomer in the gel was excised and then electrophoretically transferred into solution. Next, the rinsed Streptavidin Sepharose high performance beads were added to capture and elute out the Tau441 protein and aptamers which bound to Tau441. After 20 min incubation, beads were rinsed again with PBS buffer to remove weaker aptamer candidates. The washed beads were heated at 95° C. for 10 min, followed by a quick spin-down with a bench-top centrifuge. The surviving candidates in the supernatant were collected and were ready for PCR amplification. During this functional SELEX, we interspersed 2 rounds of regular Tau binding GE-SELEX to ensure that the DNA pool were binding only with Tau441 while minimizing the nonspecific binding towards beads. After 13 rounds selection, we obtained a pool of DNA candidates with desirable inhibitory ability as characterized by western blotting (FIG. 44D).

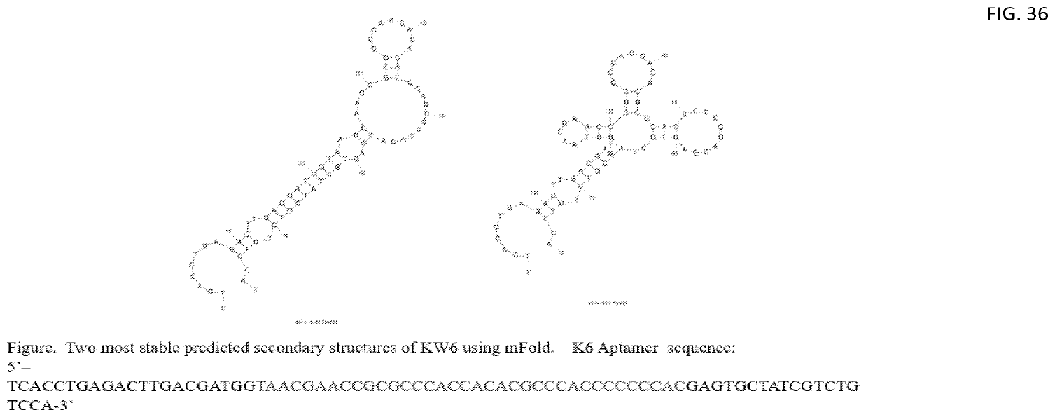

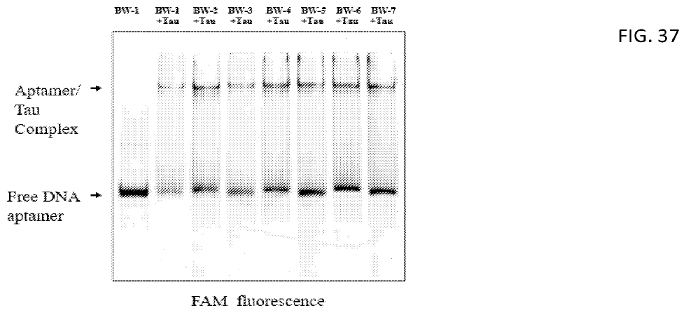

FIG. 44E shows the value of Oligo-Tau/mono-Tau intensity of quality underwent of each round of selected pool. From the first round through the ninth round, the inhibit ability of the selected pool show a stable state with the increasing rounds of selection. After nine rounds of selection, the ratio of Oli-Tau decreases dramatically and hit a low at 13^{th} rounds. However, after more two rounds selection, instead of enhancing the inhibit ability, the overall value of Oli-Tau ratio increases slightly for the 15 rounds. This indicates there is no more better inhibit ability improvement for the fourth and fifth rounds of selection. The DNA pools from the 13^{th} were cloned and sequenced after the selection. The 10 most abundant sequences were then identified in pool #13 and were then synthesized. The detailed sequence information is summarized in FIG. 34 (SEQ ID Nos: 25-44). FIG. 37 shows binding analysis of each FAM-labeled aptamer by the gel mobility shift assay. each aptamer (200 nM) was incubated with Tau protein and the complexes were separated from the free DNA on native polyacrylamide gels with electrophoresis. New aptamer/Tau caomplx bands were observed confirming several of our top candidates' ability to complex with Tau. Assay of investigate Functional SELEX DNA pool inhibiting Tau441 Oligomerization Induced by Arachidonic acid.

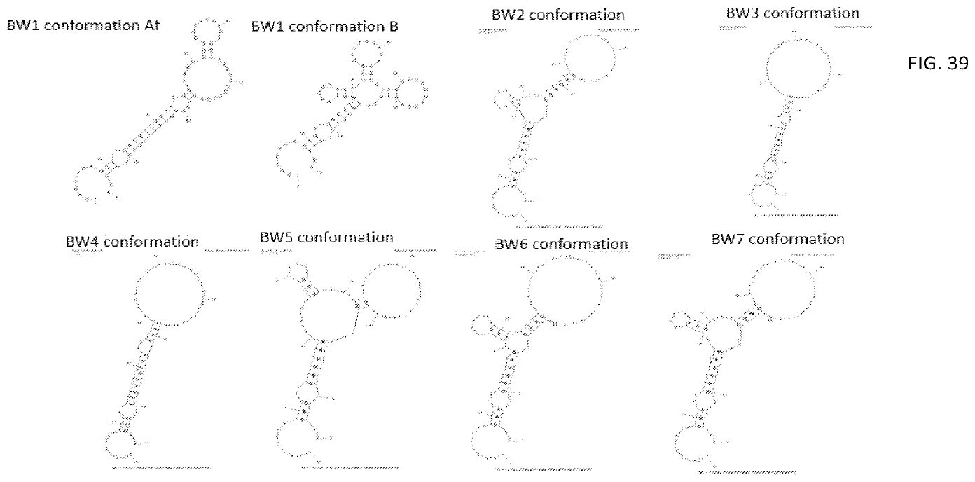

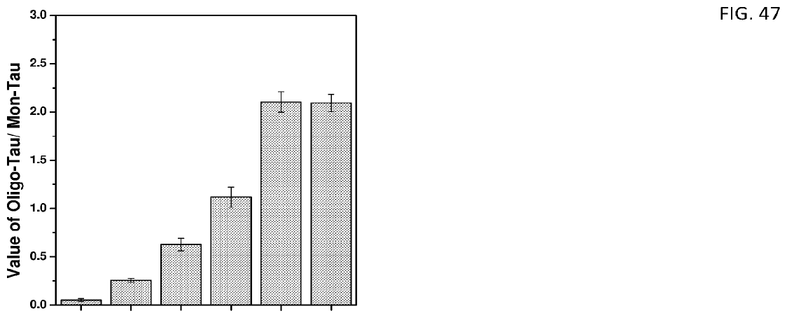

We incubated each round of DNA pools (2 μM) or DNA candidates with Tau441 (2 μM) for 30 min at room temperature in the oligomerization buffer, followed by the addition of Arachidonic acid (50 μM) for 12 h incubation at 37° C. The reaction products were analyzed by SDS-PAGE then western blotting with DA9 antibody. We found strong inhibition of Oilgo-Tau formation by the presence of BW1 (FIG. 47). FIG. 39 further shows the predicted secondary structures of BW-1 to BW-7 aptamers using mFold software.

Binding Ability and Specificity of the Selected Tau Aptamers Against Tau441 Protein.

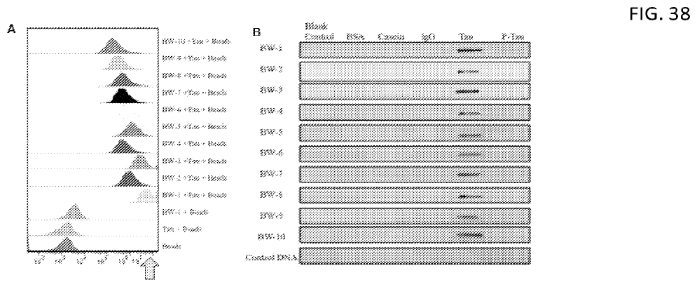

The preliminary investigation of binding ability between aptamers and Tau441 was validated by flow cytometry. Among the good aptamer candidates against Tau441, BW1 exhibited the best flowcytometry shift (FIG. 38A). Furthermore, the binding specificity of the selected tau aptamers to full-length Tau441 protein was confirmed by dot blotting by incubating each of the FAM-labeled aptamers with nitrocellulose membranes which have already been immobilized with either target Tau441 protein or nontarget proteins (FIG. 38B). Each of the tau aptamers (BW2-BW10) displayed only one main band at the native Tau position. BW1 aptamer showed one strong dot band at the tau441 position and one weak band at p-Tau position which means BW1 aptamer could also bind with p-Tau. No cross-reactivity was observed between the aptamers and the nontarget proteins, including: α casein, β casein, BSA (bovine serum albumin), and IgG (immunoglobin G). Particularly, the membranes were blocked by milk after immobilizing with each protein which further illustrated the strong specificity of the obtained aptamers.

Inhibiting the Tau Aggregation by Aptamers In Vitro.

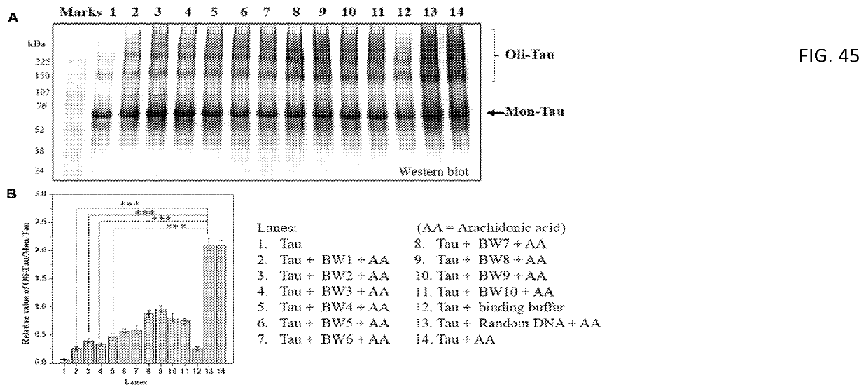

To examine the inhibitory effects of aptamer candidates on the formation of tau oligomers in vitro by arachidonic acid, Tau protein was incubated with each aptamer candidate or random DNA pool for 30 mins followed by treatment with Arachidonic acid for 12 h to form tau oligomers. As shown in FIG. 45A, the reaction products were then analyzed by SDS-PAGE and immunoblotted with polyclonal total tau antibody (DA9). In the absence of Arachidonic acid while only under the air oxidation condition, Tau441 was detected at 68K in monomeric form (lane 1) and only trace of Oli-Tau was formed in the PBS; however, a little bit more Oli-Tau were formed in the binding buffer condition (lane 12). Arachidonic acid treatment strongly caused the formation of high molecular weight oligomeric tau (lane 14). When random sequence oligonucleotide was preincubated with tau, the oligomeric assembly induction of tau was virtually unaffected. In contrast, all the tested aptamers (from BW1 to BW10) exhibit remarkable inhibitory effect on tau aggregation, among which BW1 showed the most robust inhibition on oligomeric tau formation (lane 2). As show in the FIG. 45B.

Sequence Truncation and Modification of the BW1 Aptamers.

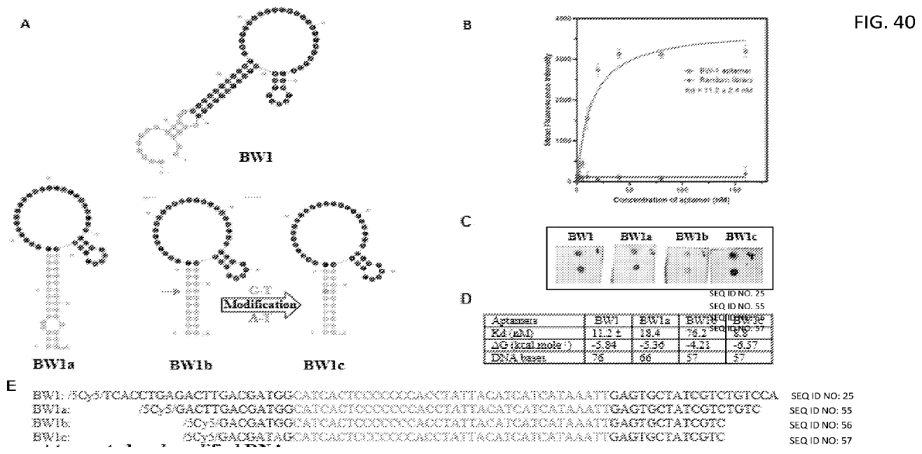

The aptamers identified are full-length sequences evolved from the initial library, which contained a fixed primer binding region on each end required in PCR amplification process. However, some full-length aptamers can be truncated into a shorter but functional sequence without compromising binding affinity towards their targets. From the assessment of the binding ability and the inhibitory ability of top ten candidates, BW1 aptamer exhibited the best binding ability and inhibitory ability, though these two aspects are not necessarily related. Aiming at BW1 aptamer, we synthesized two truncated versions of aptamer BW1, BW1a and BW1b, based on the secondary structures predicted by mFold while retaining the core secondary structure of BW1. BW1c was further modified from BW1b by replacing a G:T pair with an A:T pair in the stem structure that was envisaged to possibly enhance the stability of aptamer. (FIG. 40A). It shows that all of the engineered aptamers maintained their binding specificity to Tau441 by the dot blotting assay. (FIG. 40C). However, the weak band intensity of BW1a and BW1b represented the decreased binding affinity to Tau 441 after the truncation. However surprisingly, BW1c had stronger binding affinity than BW1a and BW1b, even better than BW1. The enhanced binding affinity indicated that the stem modified position is possibly not associated with the binding site between aptamer and Tau441. we may suggest that the stability of stem structure of aptamers is of great significance in the binding affinity. With this in mind, we proposed that several 3D structures of aptamers coexisted and were in equilibrium in the solution based on the Watson-Crick base-pairing mechanism and a relatively stable configuration would dominate the major proportion and maintain the 3D structure, making distinct contributions to binding process. It is worthwhile notifying that because the modified position located in the primer region, the successful modification could be a great inspiration in the future primer design for the SELEX research and aptamer discovery.

Inhibition of In Vitro Tau Aggregation and Tau Phosphorylation by Aptamers.

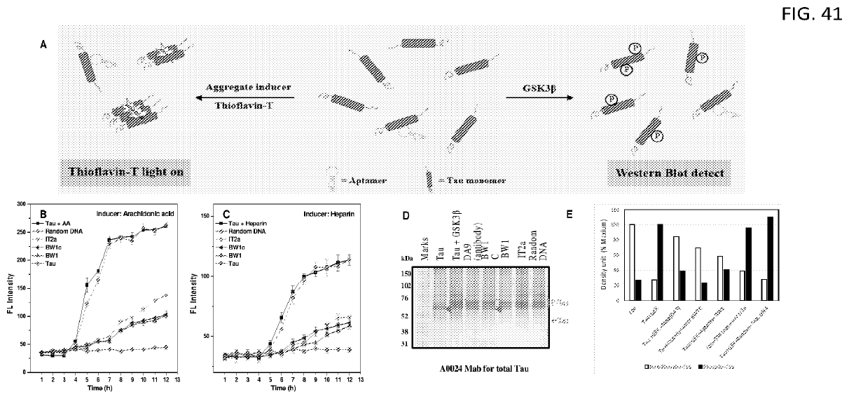

After the successful truncation and modification of the BW1 aptamer to obtain BW1c aptamer with higher affinity binding property, we then asked if the resulting aptamers would retain their inhibitory function which guided functional SELEX progress. Therefore, we investigated the inhibitory ability of BW1c together with BW1, IT2a which was developed in our formal work, in order to compare inhibitory functions of aptamers developed from binding-criteria-based SELEX vs functional SELEX. In order to characterize the inhibitory effects of aptamers, Thioflavin-T (ThT) that intercalates directly with (3-sheet structures in self-assembled, multimeric proteins such as PHFs and finally emits more intense fluorescent signals in proportion to an increasing binding magnitude, was used as an indicator of aggregation (FIG. 41A). The increasing FL signal from Tau and arachidonic acid demonstrated that arachidonic acid would greatly induce the aggregation of Tau and could be sensitively detected by ThT binding assay (q). With longer incubation time, FL signal continuously increased till reaching the plateau of FL signal where aggregation saturated. The similar increasing trend was also observed in the group of Tau, arachidonic acid with random DNA sequences indicating no inhibitory effects of random sequences. However, it showed in ThT binding assay that FL signal increased in much slower rate and weaker intensity along with the incubation time in the presence of BW1, BW1c, or IT2a aptamers, respectively, demonstrating that the formation of aggregated tau induced by arachidonic acid was significantly inhibited by BW1, BW1c, or IT2a aptamers (FIG. 41B). Excitingly, BW1 and BW1c developed from novel functional SELEX both exhibited stronger inhibition of arachidonic acid-induced aggregation than IT2a, as well as the other aggregation inducers, Heparin (FIG. 41C). It is worthwhile noticing that when the inducer was arachidonic acid, BW1 and BW1c showed similar inhibitory effect of Tau aggregate though BW1c has higher binding affinity with shorter sequence. For the Heparin, the BW1 exhibited better inhibition of aggregates indicated by lower FL intensity than BW1c in the early stage of incubation, but tends to be the same after longer incubation time up to 12 hr. Therefore, the aggregation induced by different molecules provided reliable evidence that aptamers developed from Functional SELEX exhibited better desirable function like inhibition of Tau aggregation than those from traditional binding oriented SELEX for downstream application of aptamers. Next, since tau hyperphosphorylation is intimately linked to tauopathy formation, we also examined if our Tau monomer-preferring aptamers (BW1, BW1C) can inhibit tau phosphorylation. In vitro tau phosphorylation assay was performed by incubating Tau441 (2 μM) with tau aptamers or tau-antibody (DA9 from mouse as positive control) (6 μM) for 30 mins, then recombinant protein kinase GSK3P (200 ng) was added followed by incubation for 24 h. Samples were analyzed by SDS-PAGE, followed by Western blotting with tau antibody (DAKO A0024 from rabbit). GSK3B indeed induced robust shift from non-phospho-tau to several phospho-Tau species The lower bands (blue) is the non-phosphorylated Tau while the upper bands are phospho-Tau species (induced with GSK3B-phosphoarylation (FIG. 41D). When quantification was performed on non-phospho-Tau band (white bars) and phospho-tau bands (black bars), we confirmed that GSK3B phosphorylation-mediated reduction of non-phospho-Tau levels was reversed most effectively by DA9 Tau monoclonal antibody (MAb) and by tau Aptamer BW1 and BW1c (FIG. 41E). In parallel, GSK3B phosphorylation-mediated elevations of phospho-Tau levels was reversed most effectively by DA9 Tau monoclonal antibody (MAb and by tau Aptamer BW1 and BW1c (FIG. 41E).

Inhibitory Effects of Aptamer on Tau Oligomerization in Cell Models of Tauopathy

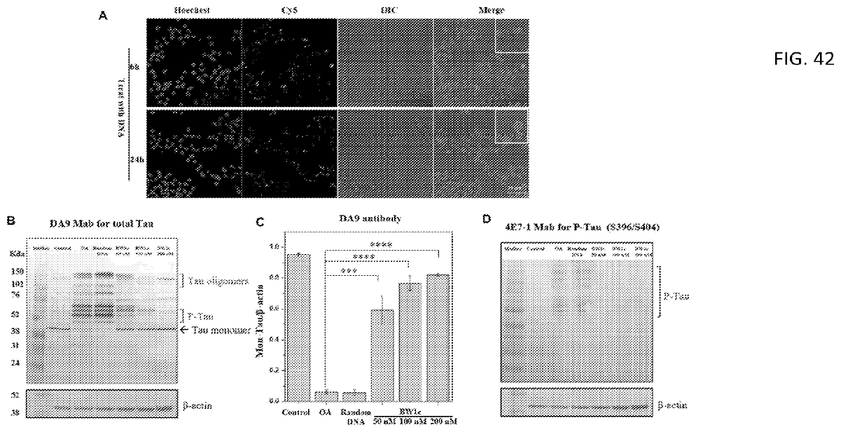

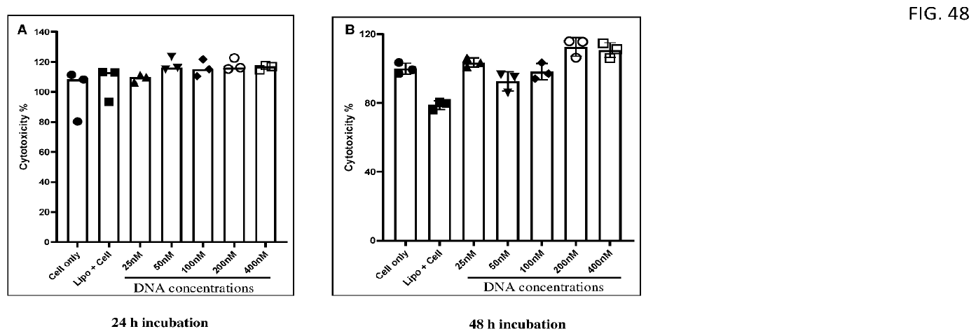

A more significant question is whether tau aptamers could inhibit Tau aggregation in intact neural cells. Therefore, to explore the possibility of applying tau aptamer to neural cell model. BW1c aptamers were transfected at different concentrations (25 nM-400 nM) using lipofectamine 2000 in neural N2a cells, a murine neuroblastoma cell line that expresses tau protein. The cell viability against lipofectamine 2000 and aptamers be measured with the MTS assay. FIG. 48 also showed that the BW1c incubation with N2a did not produce neurotoxic effects. In our investigation, confocal microscopy indicated that Cy5 labeled BW1c efficiently entered the cytoplasm of N2a cells in the presence of lipofectamine 2000 (FIG. 42A). Others and we have developed and validated cell models of tauopathy by exploiting the use of Okadaic acid (OA) a protein phosphatase 1 and 2A (PP1/PP2A) inhibitor for inducing tau hyperphosphorylation/pathological state in cell-based such as N2a cells. It has also been reported that OA inhibition of tau phosphatases allows the activation of multiple tau kinases, eventually leading to tau hyperphosphorylation. It has been shown that the OA treatment in wild-type mice causes tauopathy-related abnormality in different regions of the brain. In our neural cell model of tauopathy, N2a cells were treated with OA (100 nM) for 24 h to induce tau hyperphosphorylation and oligomerization, thus mimicking a tauopathy relevant condition. Following OA-treatment of N2a cell mode, Total cellular proteins extracted from cells were analyzed by using western blotting with DA9 antibody which specifically binds with total tau for the identification and quantification of Monomeric-Tau (Mono-Tau, 46 kDa), p-Tau (50-70 kDa) and Oli-gomericTau (Oligo-Tau 90-160 kDa), respectively, and P-Tau (Ser396/S404) monoclonal antibody bound with p-Tau further indicats the presence of p-Tau in electrophoretic analysis (FIG. 42B, C, D). Using total Tau antibody DA9 it is found that with addition of BW1c aptamer the band intensity and Tau oligomers were much weaker than OA alone or OA+random DNA, indicating that Tau oligomerization in this cellular model was strongly inhibited by BW1c in dose-dependent manner (FIG. 42 B, C). We also suspected that these OA induced Tau species with higher apparent molecular weight and oligomeric forms of Tau likely contain phospho-Tau. Using immunblotting with P-Tau (Ser396/S404) monoclonal antibody, we confirmed that OA treatment indeed produced multiple bands of P-Tau MAb-positive tau and tau oligomers (FIG. 42D). Importantly, BW1c treatment, but not random DNA, dose-dependently (50 nM to 200 nM) diminished overall P-Tau band signals to close to baseline levels.

In summary, our unparalleled findings so far demonstrated, for the first time, the feasibility of the concept of Functional SELEX for selecting functional molecular probes. The innovative SELEX strategy provided a flexible, efficient and straightforward platform for systematically discovering functional aptamers beyond binding. The emerging aptamer panels comprised of molecules with great potential in binding and inhibiting properties against neuroprotein aggregations that are associated with brain pathologies. The “Functional SELEX” approach we demonstrated here established a framework for the rapid and biofunction-specific screening and discovery of a new class aptamers with the potential of targeting a wide range of molecules that undergo disease state-dependent biofunction changes (such as gain-of-function or loss-of-function).

### Methods Related to Example 16

GE SELEX Procedures.

The GE-SELEX was performed by using the DNA library to target Tau-441 protein. Synthetic single stranded DNA, consisting of 36 randomized nucleotides flanked by two constant regions was used as the starting pool. The DNA bound to Tau-441 was separated from unbound DNA on a 8% Native PAGE gel at 4° C. by gel electrophoresis. The shifted band, which corresponded to the DNA-Tau441 complex, was excised, heated to elute out DNA which were then followed by PCR amplification in a reciprocal manner. The DNA band patterns on the gels were recognized by labeled FAM fluorescence and imaged with a Typhoon Imaging System (Amersham Biosciences). In the first round, an initial DNA library consisting of 20 nmol of the randomized oligonucleotides was used as a starting pool. Non-SELEX procedure was employed in the first-round gel Selex: repeat the gel separation for DNA and DNA-Tau complex before the PCR amplification. For later rounds, 20 pmol to 100 pmol of samples amplified from previously recovered survivors were used. In each round, the DNA pool was heated at 95° C. for 3 min, followed by rapid cooling on ice for 5 min before starting incubation, allowing the DNA sequences to fold into the most favorable secondary structures. All incubations were performed at 4° C. The number of optimized PCR amplification cycles for each round was confirmed with agarose gel electrophoresis. Streptavidin Sepharose high performance beads (GE Healthcare Life Sciences) were used to isolate the PCR products from the reaction mixture. The fluorophore-labeled amplicons were then dissociated from the biotinylated antisense DNA by washing with 20 mM NaOH. Finally, the ssDNA was desalted with a NAP-5 column (GE Healthcare Life Sciences). The entire process is summarized in Table 3.

TABLE 3
In vitro selection conditions for GE-SELEX.

| | | | | | Gel electrophoresis | PCR |
|---|---|---|---|---|---|---|
| Round | DNA | | Protein | | (Running time/Temp.) | cycles |
| 1 | 20 | nmol | 1 | μg | 8% Native | 18 |
| | | | | | (50 min/4° C.) | |
| 2 | 100 | pmol | 0.1 | μg | 8% Native | 16 |
| | | | | | (150 min/25° C.) | |
| 3 | 20 | pmol | 0.02 | μg | 8% Native | 18 |
| | | | | | (150 min/25° C.) | |

Functional SELEX Procedures.

In the first step, we preincubated DNA pools (4 μM or 6 μM) with biotin labeled Tau441 (2 μM) for 30 min at room temperature in the oligomerization buffer (10 mM HEPES, 100 mM NaCl, 10 mM MgCl₂, 50 mM KCl, 1 mM EDTA, 1 mM DTT), followed by the addition of Arachidonic acid (50-100 μM) for 12 h incubation at 37° C. The Monomer Tau441 was separated from Oligo-Tau441 by gel electrophoresis on an 8% Native PAGE gel at 4° C., running for 150 min at 140V. The Monomer Tau441 position gel was excised and electrophoretically transferred into solution. Then added Streptavidin Sepharose high performance beads which were washed with 1 mL of PBS three times before use to capture the Tau441 protein and aptamers which bind with Tau441. After incubating for 20 min, beads were washed with buffer to remove weaker candidates. The treated beads were then heated at 95° C. for 10 min, followed by quick spin-down with a centrifuge. The surviving candidates in the supernatant were collected and ready for PCR amplification.

TABLE 4
In vitro selection conditions for Functional SELEX.

| | | | | |
|---|---|---|---|---|
| | DNA | | Arachidonic | PCR |
| Round | (μM) | Protein(μM) | acid (μM) | cycles |

| 1 | 4 | μM | 2 | μM | 50 | μM | 18 |
|---|---|---|---|---|---|---|---|
| 2 | 4 | μM | 2 | μM | 50 | μM | 16 |
| 3 | 4 | μM | 2 | μM | 60 | μM | 18 |
| 4 | 4 | μM | 2 | μM | 60 | μM | 24 |
| 5 | 6 | μM | 0.1 | μM | 0 | | 20 |
| 6 | 4 | μM | 2 | μM | 50 | μM | 18 |
| 7 | 4 | μM | 2 | μM | 50 | μM | 16 |
| 8 | 4 | μM | 2 | μM | 60 | μM | 22 |
| 9 | 4 | μM | 2 | μM | 60 | μM | 20 |
| 10 | 6 | μM | 0.1 | μM | 0 | | 18 |
| 11 | 4 | μM | 2 | μM | 80 | μM | 16 |
| 12 | 4 | μM | 2 | μM | 100 | μM | 20 |
| 13 | 4 | μM | 2 | μM | 80 | μM | 16 |

DNA Library and Primers.

The forward and reverse primers were labeled with FAM and biotin, respectively, at their 5′-ends. The sequence of the forward primer was 5′-FAM-TCA CCT GAG ACT TGA CGA TGG-3′ while that of the reverse primer was 5′-Biotin-TGG ACA GAC GAT AGC ACT C-3′. The DNA library consisted of a randomized 36-nt region flanked by primer binding sites: 5′-TCACCTGAGACTTGACGATGG-(N)36-GAGTGCTATCGTCTGTCCA-3′. All DNA templates and primers were purchased from Integrated DNA Technologies and purified by reverse phase HPLC.

Polymerase Chain Reaction (PCR).

PCR parameters were optimized before the selection process. The Tm of the primers was optimized from 55° C. to 64° C. using standard PCR parameters from manufacture's guide for the DNA polymerase, MgCl₂, and dNTP concentrations. 58° C. was determined as the optimum annealing temperature. All PCR mixtures contained 50 mM KCl, 10 mM Tris-HCl (pH 8.3), 1.5 mM MgCl₂, 0.2 mM each dNTP, 0.5 μM each primer, and Hot Start Taq DNA polymerase (0.015 units/μL). PCR was performed on a BioRad C1000 Thermo Cycler, and all PCR reagents were purchased from Takara. The amplification began with a hot start at 95° C. for 90 s to activate Taq DNA polymerase. Then each of the repeated amplification cycles was performed at 95° C. for 10 s, 58° C. for 30 s, and 72° C. for 30 s.

Real-Time Polymerase Chain Reaction (RT-PCR).

Amplification plot for a 10-fold diluted random library template series by real-time PCR which employed the NEB Luna Universal qPCR Master 2× reaction Mix (contains SYBR Green). The amplification began with a hot start at 95° C. for 90 s to activate Taq DNA polymerase. Then each of the repeated amplification cycles was performed at 95° C. for 5 s, 58° C. for 30 s, and 72° C. for 30 s. No template control (NTC) shows a positive signal after 30 cycles indicating the formation of primer dimer (FIG. 1A). Real-time analyses of all methods tested were performed on a Roche Light Cycler 480.

Flow Cytometric Analysis of DNA.

BD Accuri C6 flow cytometry (BD Immunocytometry Systems) was used to monitor and evaluate the binding ability and specificity of the selected candidates during the selection process. For each sample, 10 μL of bare beads (˜7×10⁴ beads) were washed with 500 μL of PBS three times. After removal of the supernatant, Beads were incubated and suspended in 500 μL Tau protein solution (1 μM) for 1 h at 4° C. Then the beads were washed with 500 μL of PBS three times. Then beads were resuspended in 80 μL of buffer containing DNA pool to be tested at 250 nM for 30 min. When examining the saturation of fluorescent signal with the selection pools, the DNA concentration used was decreased to 100 nM instead. Incubations were carried out at 37° C. unless stated otherwise. Afterward, beads were washed against 500 μL of buffer twice and then suspended in 100 μL of buffer for flow cytometric analysis.

Next-Generation Deep Sequencing of DNA Survivors.

Functional SELEX enriched DNA pools from rounds #13 which amplified with primers without biotin and FAM were submitted for sequencing. Each amplicon from DNA pool was barcoded by the TruSeq DNA library preparation kit (Illumina) separately and then submitted to Illumina next-generation DNA sequencing. Further analysis of the sequencing results was finished by in-house software.

Chemical Synthesis and Purification of Aptamer Candidates.

The 10 most abundant sequences from round #13 were synthesized and finally obtained from Integrated DNA Technologies.

Semi-Quantification of Specificity of Selected Tau Aptamers Against Tau441 Protein by Dot Blotting.

The nitrocellulose membranes were socked in methanol till it becomes wet (1 min) then the membranes were washed with TBST solution for 5 mins. Tau protein (stripped band 0.5 μg) were dripped on the NC membranes in every dot and then waited to dry (2 mins). The membranes were blocked with 1% fat-free milk in TBST for 1 hour and were respectively incubated with series of heat denatured aptamers (250 nM) for 1 hour in blocking buffer (contain 0.5 mM Mg²⁺) at room temperature. Next the membrane was washed for three times by TBST, 5 min for each time. Dots were scanned simultaneously by a Typhoon Imaging System (Amersham Biosciences). When investigate the specificity of BW1 series aptamers Tau441 protein (0.05 μg) were dripped on the NC membranes and aptamer concentration turn to 100 nM.

Binding Analysis of Each FAM-Labeled Aptamer by the Gel Mobility Shift Assay.

Except for the first channel, each aptamer (200 nM) was incubated with Tau protein (20 μg/mL) at 37° C. for 30 min, and the complexes were separated from the free DNA on native 8% polyacrylamide gels with running at 25° C. and 145 V for 40 min. The gels were scanned and imaged by using a Typhoon Imaging System (Amersham Biosciences)

Investigate Arachidonic Acid-Induced Tau Aggregation.

Tau441 (2 μM) and Arachidonic acid (0-100 μM) were incubated together in the oligomerization buffer (10 mM HEPES, 100 mM NaCl, 10 mM MgCl₂, 50 mM KCl, 1 mM EDTA, 1 mM DTT) with constant agitation at 37° C. Arachidonic acid (100 mM) were prepared in 100% ethanol prior to use. The reaction products were analyzed by SDS-PAGE electrophoresis on a 4-20% precast-gels (Bio-Rad) at 140V for 90 mins. Then the gel was washed in water for 20 min, stained with Bio-safe Coomassie stain for 60 min, then destained in water for 90 min (change the water every 30 mins). The gel was scanned by Typhoon Imaging System (Amersham Biosciences). Tau and oligomer Tau were also imaged by western blotting after aggregation. Note: when the concentration of AA lower than 75 μM, the degree of shaking would affect the aggregate reaction. However, if the reaction was conducted without shaking or rocking, the aggregation reaction was found out to be finally delayed. Besides, the higher tau concentration (more than 5 μM) could facilitate rapid nucleation and resulted in the formation of more oligomers.

Assay of Investigate Functional SELEX DNA Pool Inhibiting Tau441 Oligomerization Induced by Arachidonic Acid.

We incubated each round of DNA pools (2 μM) or DNA candidates with Tau441 (2 μM) for 30 min at room temperature in the oligomerization buffer, followed by the addition of Arachidonic acid (50 μM) for 12 h incubation at 37° C. Arachidonic acid (100 mM) were prepared in 100% ethanol prior to use. The aptamers were prepared in 1×PBS with 5 mM MgCl₂. The reaction products were analyzed by SDS-PAGE then western blotting with DA9 antibody.

ThT Fluorescence Assay of Detect Tau Aggregation.

In vitro tau protein (2 μM) was premixed with or without aptamer (6 μM) for 30 mins then add aggregation inducer (Arachidonic acid, heparin or GSK3β), then incubated for 12 h with constant agitation at 37° C. For each aggregation reaction condition: (a). Arachidonic acid reaction concentration: Tau protein: 2 μM, Aptamer: 6 μM, Arachidonic acid: 75 μM. HEPES: 10 mM, NaCl: 100 mM, MgCl₂ 10 mM, KCl: 50 mM, EDTA: 1 mM, DTT: 1 mM, THT: 5 mM. (b). Heparin reaction concentration: Tau protein: 2 μM, Aptamer: 6 μM, heparin 50 μM, HEPES: 10 mM, NaCl: 100 mM, MgCl₂ 10 mM, KCl: 50 mM, EDTA: 1 mM, DTT: 1 mM, THT: 5 mM. (C) GSK3β reaction concentration: Tau protein: 2 μM, Aptamer: 6 μM, GSK3β: 50 μM. ATP: 6 μM, HEPES: 10 mM, NaCl: 100 mM, MgCl₂ 10 mM, 50 mM KCl, THT: 5 mM. ATP: 6 μM. The aggregation of tau was monitored by detecting the fluorescent of thioflavin T (ThT) in Greiner Bio-One 384-well micro-plates using a Biotek Synergy 2 microplate reader. Excitation wavelength: the excitation and emission filters were 450/15 and 485/15 nm.

Cell Lines and Cell Culture Media

Mouse neuroblastoma N2a cell were commercially available from American Type Culture Collection Company (ATCC1 CCL-131™, Manassas, Va., USA). The complete culture media were mixture 1:1=DMEM (Dulbecco's modified Eagle medium): Opti-MEM (reduced serum Eagle's minimum essential media) and supplemented with 5% FBS (Thermo-Fisher), 100 units/ml penicillin and 0.1 mg/ml streptomycin. Cells were incubated at 37° C. in a humidified 5% CO₂-containing atmosphere. Endogenous tau was used to assess the tau phosphorylation levels and analysis.

Lipofectamine-2000 Mediated Aptamer Treatment,

N2a cells (2×10⁴ cells per well 200 μL) were seeded into 48-well plate and incubated overnight until they reached 80% confluency. SNJ-1945 (S, 100 μM) and Z-DCB (Z, 60 μM) were added to all experimental conditions before the treatment for 1 h. The cells were washed with 1×PBS. A 200 μL mixture containing 0.4 μL Lipofectamine-2000 (Invitrogen, Carlsbad, Calif.), aptamer (25-400 nM) in DMEM were added into each well and incubated at 37° C. in a humidified incubator for 1 hour. The DNA/Lipofectamine-2000 complexes were discarded and DMEM media was added to the dishes. Then culture for 1 hour. Next, okadaic acid (OA; 100 nM) was added culture for 24 h in DMEM media.

Cell Lysate Collection and Preparation

The aptamer-transfected cells in the culture plate were collected and washed with 1×PBS, then incubated in lysis buffer for 90 minutes at 4° C., then centrifuged at 15,000 rpm for 15 minutes to remove cell debris. The Triton-X lysis buffer including: 1 mM DTT, 1% phosphatase inhibitors (Sigma), 1% Mini-Complete protease inhibitor cocktail tablet (Roche Biochemicals), and 1% Triton X-100.

Western Blotting Protocol.

When the sample were pure protein, after running the SDS-PAGE gel, proteins were transferred from the gel to the PVDF membrane which was activated with methanol for 1 min and rinse with transfer buffer before preparing the stack. Transfer the protein under voltage (20V) for 30 mins. Take out the membrane then block the membrane with milk for 1 h at room temperature. Then incubate the membrane with primary antibody total tau monoclonal DA9 (a.a. 102-140) in blocking buffer at 4° C. overnight. Wash the membrane three times with TBST (20 mM Tris-HCl, 500 mM NaCl, 0.05% Tween20, pH7.5) for 10 min each. Incubate the membrane with the phosphatase conjugated secondary antibody in blocking buffer at room temperature for 1 h. Wash the membrane again for three times wiht TBST for 10 min each. Immunoreactive bands were detected by developing with nitro blue tetrazolium and 5-bromo-4-chloro-3′-indolylphosphate (NBT/BCIP) (KPL). Quantitative evaluation of protein levels was performed via computer-assisted densitometric scanning (NIH ImageJ, version 1.6). For the Cell lysate sample, the concentrations of proteins from cell lysates were determined by bicinchoninic acid (Pierce Inc., Rockford, Ill., USA) microprotein assays against albumin standards. Equal protein samples (20 μg) were prepared for SDS-PAGE electrophoresis gels. In the process of western blotting analysis, total tau monoclonal antibody DA9 (a.a.102-140), monoclonal phospho-tau (p-tau) antibodies were 4E7-1 Mab for P-Tau (pS396/pS404) were used as primary antibody treatment overnight. The (3-actin antibody was used as the internal control for the protein loading. Next treat with secondary antibody and detect the immunoreactive bands same with protein sample treatment. A rainbow molecular weight marker (RPN800E, GE Healthcare, Bio-Sciences, Pittsburgh, Pa., USA) form 14 kDa to 250 kDa was used to estimate the molecular weight of each band.

Confocal Microscopy and Flow Cytometry Analysis Aptamer Internalization

N2a cells (2×10⁴ cells/well) were seeded in a 4-chamber confocal dish overnight. Then the cells were washed with 1×PBS. A 200 μL mixture containing 0.4 μL Lipofectamine-2000 (Invitrogen, Carlsbad, Calif.), Aptamer (25-400 nM) in DMEM was added into dish and incubated at 37° C. in a humidified incubator for 1 hour. The DNA/Lipofectamine-2000 complexes were discarded and DMEM media was added to the dishes. Then the cells were incubated for 6 h and 24 h. Afterwards, the cells were incubated with Hoechst for nuclear staining 10 mins, followed by washing with 1×PBS three times. The fluorescence images were analyzed by using a Leica TCS SP5 confocal microscope with a 63× oil objective at 37° C. Fluorescence Cy5 images were acquired using 651 nm excitation and 670 nm emission. The Hoechst images were acquired using 392 nm excitation and 440 nm emission.

Cell Viability Study

The cytotoxicity of Lipofectamine-2000 Mediated Aptamer (25 nM-400 nM) treatment were evaluated by MTS assay. N2a Cells (2×10⁴ cells/well) were seeded in 24-well plates overnight. Then, the cells were incubated with lipofectamine-2000/aptamer (25 nm-400 nm) at 37° C. in 5% CO₂ for 1 h. Then, the supernatant was discarded followed by washing with 1×PBS three times. Full culture medium (500 μL) were added and incubated for 24 h/48 h. Then the supernatant was discarded, MTS (20 μL CellTiter reagent) diluted in complete culture media (200 μL) was added to every well then incubated for 1 h. Next step, the sample's 490 nm absorbance was acquired by a CLARIOstar plate reader (BMG LABTECH) and normalized to the untreated control cells.

Measurement of Binding Kinetics/Affinities of the Selected Aptamers.

The binding affinities/kinetics of the selected aptamers were measured on an Octet QKe system (Fortebio). All measurements were performed on 96-well microplates that were agitated at 1000 rpm at 30° C. The streptavidin dip sensors were incubated in PBS solution to build baseline 1 for 10 mins. Then the sensors were incubated in biotin tag Tau441 protein (SignalChem) with a concentration of 1 μg/mL for 10 min. Followed the sensors were brought into fresh PBS buffer to establish baseline 2 for another 10 min. For kinetics analysis, the Tau protein-immobilized sensors were transferred to the wells containing aptamer dilutions (1 μM, 500 nM, 250 nM, 125 nM, 62 nM) for the association step (10 min) and then moved to the wells with fresh PBS buffer (PBS with 0.5 mM Mg²⁺) for the dissociation step (10 min). All the aptamers were dissolved in PBS with 0.5 mM Mg²⁺. Two reference sensors were used, one treated with PBS solution instead of aptamer solution, and another was treated with control aptamer (Sgc-8 aptamer, 6 μM). All other steps remained the same. Association rate constants (kon), dissociation rate constants (kdis), and equilibrium dissociation constants (Kd) for each aptamer binding to target Tau441 were calculated using the ForteBio data analysis software 10.0.

Statistical Analysis.

Data were presented as mean±SEM for n=3. Statistical analysis was performed with two-way ANOVA Tukey's Test. For multiple comparisons, one-way ANOVA, followed by the Bonferroni's post hoc test, was performed. *p<0.05, **p<0.01, ***p<0.001 and ****p<0.0001, ns: nonsignificant.

### REFERENCES

All references listed below and throughout the specification are hereby incorporated by reference in their entirety.
1. Buée, et al., Tau protein isoforms, phosphorylation and role in neurodegenerative disorders. Brain Res. Rev. 33:95-130, 2000.
2. Chakravarthy et al., Development of DNA aptamers targeting low-molecular-weight amyloid-β peptide aggregates in vitro. Chem. Commun. 54:4593-4596, 2018.
3. Chang et al., Untangling the tauopathy for Alzheimer's disease and Parkinsonism. J. Biomed. Sci. 25:54, 2018.
4. Chi-hong et al., Aptamer-based endocytosis of a lysosomal enzyme. Proc. Natl. Acad. Sci. 105:15908-15913, 2008.
5. Cisek et al., Structure and mechanism of action of tau aggregation inhibitors. Curr. A. lzheimer Res. 11: 918-927, 2014.
6. Goedert and Spillantini, Propagation of Tau aggregates. Mol. Brain 10 (1):18, 2017.
7. Götz et al., Tau-targeted treatment strategies in Alzheimer's disease. Br. J. Pharmacol. 165:1246-1259, 2012.
8. Guo et al., Roles of tau protein in health and disease. Acta neuropathologica 133:665-704, 2017.
9. Hu et al., Expression of tau pathology-related proteins in different brain regions: a molecular basis of tau pathogenesis. Front. Aging Neurosci. 9:311. 2017.
10. Iqbal et al., Tau and neurodegenerative disease: the story so far. Nat. Rev. Neurol. 12:15, 2016.
11. Jeong and Na, Synthesis of a gadolinium based-macrocyclic MRI contrast agent for effective cancer diagnosis. Biomater. Res. 22 (1): 17, 2018.
12. Jiang et al., Supramolecularly engineered circular bivalent aptamer for enhanced functional protein delivery. J. Am. Chem. Soc. 140:6780-6784, 2018.
13. Jost et al., Penetration and distribution of gadolinium-based contrast agents into the cerebrospinal fluid in healthy rats: a potential pathway of entry into the brain tissue. Eur. Radiol. 27 (7):2877-2885, 2017.
14. Kuai et al., Circular bivalent aptamers enable in vivo stability and recognition. J Am. Chem. Soc. 139:9128-9131, 2017.
15. Kwon et al., Stress and traumatic brain injury: a behavioral, proteomics, and histological study. Front. Neurol. 2:12, 2011.
16. Li et al., Permeability of endothelial and astrocyte cocultures: in vitro blood-brain barrier models for drug delivery studies. Ann. Biomed. Engineer. 38:2499-2511, 2010.
17. Liu et al., In vitro and in vivo studies on the transport of PEGylated silica nanoparticles across the blood-brain barrier. ACS Appl. Mater. Inter. 6:2131-2136, 2014.
18. Macdonald et al., Truncation and mutation of a transferrin receptor aptamer enhances binding affinity. Nucl. Acid Ther. 26:348-354, 2016.
19. Macdonald et al., Development of a bifunctional aptamer targeting the transferrin receptor and epithelial cell adhesion molecule (EpCAM) for the treatment of brain cancer metastases. ACS Chem. Neurosci. 8:777-784, 2017.
20. Mazanetz and Fischer, Untangling tau hyperphosphorylation in drug design for neurodegenerative diseases. Nat. Rev. Drug Disc. 6:464, 2007.
21. Mietelska-Porowska et al., Tau protein modifications and interactions: their role in function and dysfunction. Int. J. Mol. Sci. 15:4671-4713, 2014.
22. Omidi et al., Evaluation of the immortalised mouse brain capillary endothelial cell line, b. End3, as an in vitro blood-brain barrier model for drug uptake and transport studies. Brain Res. 990:95-112, 2003.
23. Pang et al., Bioapplications of cell-SELEX-generated aptamers in cancer diagnostics, therapeutics, theranostics and biomarker discovery: a comprehensive review. Cancers 10:47, 2018.
24. Patel and Patel, Crossing the blood-brain barrier: recent advances in drug delivery to the brain. CNS Drugs 31:109-133, 2017.
25. Paterson and Webster, Exploiting transferrin receptor for delivering drugs across the blood-brain barrier. Drug Discovery Today: Technologies 20:49-52, 2016.
26. Rubenstein et al., Novel Mouse Tauopathy Model for Repetitive Mild Traumatic Brain Injury: Evaluation of Long-Term Effects on Cognition and Biomarker Levels After Therapeutic Inhibition of Tau Phosphorylation. Front. Neurol. 10, 2019.
27. Saha and Sen, Tauopathy: A common mechanism for neurodegeneration and brain aging. Mech. Ageing Development 2019.
28. Santacruz et al., Tau suppression in a neurodegenerative mouse model improves memory function. Science 309:476-481, 2005.
29. Shamili et al., Immunomodulatory properties of MSC-derived exosomes armed with high affinity aptamer toward mylein as a platform for reducing multiple sclerosis clinical score. J. Controlled Release 299:149-164, 2019.
30. Teng et al., Identification and characterization of DNA aptamers specific for phosphorylation epitopes of tau protein. J. Am. Chem. Soc. 140:14314-14323, 2018.
31. Thelin et al., A review of the clinical utility of serum S100B protein levels in the assessment of traumatic brain injury. Acta Neurochirurgica 159:209-225, 2017.
32. Tian et al., Targeted imaging of brain tumors with a framework nucleic acid probe. ACS Appl. Mater. Inter. 10:3414-3420, 2018.
33. Wang et al., Imaging of Neurite Network with an Anti-L1CAM Aptamer Generated by Neurite-SELEX. J. Am. Chem. Soc. 140:18066-18073, 2018.
34. Wang and Mandelkow, Tau in physiology and pathology. Nat. Rev. Neurosci. 17:22, 2016.
35. Wong et al., Review of Current Strategies for Delivering Alzheimer's Disease Drugs across the Blood-Brain Barrier. Int. J. Mol. Sci. 20:381, 2019.
36. Xiong et al., Animal models of traumatic brain injury. Nat. Rev. Neurosci. 14:128. 2013.
37. Yang et al., Wang, K. K., Dual vulnerability of TDP-43 to calpain and caspase-3 proteolysis after neurotoxic conditions and traumatic brain injury. J. Cerebral Blood Flow & Metab. 34 (9):1444-1452, 2014.
38. Yang et al., Temporal MRI characterization, neurobiochemical and neurobehavioral changes in a mouse repetitive concussive head injury model. Sci Rep-Uk 5:11178, 2015.
39. Yang et al., Identification of two immortalized cell lines, ECV304 and bEnd3, for in vitro permeability studies of blood-brain barrier. Plos One 12:e0187017, 2017.
40. Yu and Watts, Developing therapeutic antibodies for neurodegenerative disease. Neurother. 10:459-475, 2013.
41. Zheng et al., Novel DNA Aptamers for Parkinson's Disease Treatment Inhibit α-Synuclein Aggregation and Facilitate its Degradation. Mol. Ther. Nucl. Acids 11: 228-242, 2018.
42. Zhou and Rossi, Aptamers as targeted therapeutics: current potential and challenges. Nat. Rev. Drug Disc. 16:181, 2017.

1. A bispecific circularized DNA aptamer comprising:
(a) a first oligonucleotide aptamer that binds to Tau protein; and
(b) a second oligonucleotide aptamer that binds to a second protein, where the first oligonucleotide is ligated to the second oligonucleotide and the DNA aptamer penetrates the blood-brain barrier.

2. The bispecific DNA aptamer of claim 1, wherein the second protein is TfR, L1CAM, GLAST-1, or ACSA-2.

3. The bispecific DNA aptamer of claim 1, which is coupled to a signaling moiety.

4. The bispecific DNA aptamer of claim 3, wherein the signaling moiety is a molecular beacon, fluorescent tag, or a radioisotope for detection by positron emission tomography (PET), single-photon emission computed tomography (SPECT), and/or contrast-agent-based MRI.

5. The bispecific DNA aptamer of claim 3, wherein the signaling moiety is dodecane tetraacetic acid (DOTA) or DOTA complexed to Gd⁺³.

6. (canceled)

7. A DNA aptamer comprising:
(a) a DNA Tau aptamer; and
(b) a protein aggregate binding moiety.

8. A DNA aptamer of claim 7, wherein the DNA Tau aptamer is IT2a.

9. A DNA aptamer of claim 7, wherein the DNA Tau aptamer is a bispecific aptamer according to claim 1.

10. A DNA aptamer of claim 7, wherein the protein aggregate binding moiety is methylene blue.

11. A trispecific circularized DNA aptamer comprising:
(a) a first oligonucleotide aptamer that binds to Tau protein; and
(b) a second oligonucleotide aptamer that binds to a second protein,
(c) a third oligonucleotide aptamer that binds to a third protein,
where the three oligonucleotide aptamers are ligated and annealed to each other to form a y-shape, and the DNA aptamer penetrates the blood-brain barrier.

12. The trispecific DNA aptamer of claim 11, wherein the second protein are TfR.

13. The trispecific DNA aptamer of claim 11, wherein the third protein comprises L1CAM, GLAST-1, or ACSA-2.

14. The trispecific DNA aptamer of claim 11, wherein a 3′ end of the oligonucleotide aptamer contains an elongated stem that is ligated to a 5′ end of one other oligonucleotide aptamer for formation of the y-shape where all three oligonucleotide aptamers are bound together.

15. The trispecific DNA aptamer of claim 14, wherein the elongated stems have complementary sequences that hybridize for formation of the y-shape.

16. The trispecific DNA aptamer of claim 11, which is coupled to a signaling moiety.

17. The trispecific DNA aptamer of claim 16, wherein the signaling moiety is a molecular beacon, fluorescent tag, or a radioisotope for detection by positron emission tomography (PET), single-photon emission computed tomography (SPECT), and/or contrast-agent-based MRI.

18. The trispecific DNA aptamer of claim 16, wherein the signaling moiety is dodecane tetraacetic acid (DOTA) or DOTA complexed to Gd⁺³.

19. An aptamer comprising a sequence selected from any of SEQ ID NOs: 1-15, 23 or 24.

20. An aptamer comprising a sequence selected from any of SEQ ID NOs: 1-15, 25-44, and 55-57.

21. (canceled)

22. An aptamer comprising a sequence selected from any of SEQ ID NOs: 45-54.

23-29. (canceled)

30. A method of treating tauopathy in a subject in need, comprising administering to the subject an aptamer, wherein the aptamer comprises a sequence selected from any of SEQ ID NOs:1-15, 23-44, or 55-57.

31. (canceled)

32. (canceled)

33. (canceled)

34. (canceled)

35. (canceled)
